SEQUENCE LISTING (94 sequences)

SEQ ID NO: 1 - protein, 107 aa, synthetic construct [sequence omitted]

SEQ ID NO: 2 - protein, 121 aa, synthetic construct [sequence omitted]

SEQ ID NO: 3 - protein, 214 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 451 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 108 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 6 - protein, 126 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 7 - protein, 215 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 456 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 115 aa, synthetic construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 10 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 11 - protein, 117 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 12 - protein, 217 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 447 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 107 aa, synthetic construct [sequence omitted]

SEQ ID NO: 15 - protein, 116 aa, synthetic construct [sequence omitted]

SEQ ID NO: 16 - protein, 214 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 446 aa, synthetic construct
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 209 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 19 - protein, 455 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 451 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 205 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 22 - protein, 455 aa, synthetic construct
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 456 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 205 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 25 - protein, 460 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 447 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 206 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 28 - protein, 465 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 446 aa, synthetic construct
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 709 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGGGGSGGGGSGGGGSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 31 - protein, 704 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKGGGGSGGGGSGGGGSQVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSS

SEQ ID NO: 32 - protein, 714 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGGGGSGGGGSGGGGSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 33 - protein, 709 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKGGGGSGGGGSGGGGSQVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSS

SEQ ID NO: 34 - protein, 705 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 35 - protein, 711 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGGSGGGGSGGGGSQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 36 - protein, 704 aa, synthetic construct
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 37 - protein, 710 aa, synthetic construct
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGGSGGGGSGGGGSQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 38 - protein, 340 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 583 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSGGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 336 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 582 aa, synthetic construct
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSGGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 341 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGGGGSGGGGSGGGGSEIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 588 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSGGGGSGGGGSGGGGSQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 337 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKGGGGSGGGGSGGGGSEIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 587 aa, synthetic construct
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSGGGGSGGGGSGGGGSQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 339 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGGSGGGGSGGGGSQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 47 - protein, 583 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 340 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGGSGGGGSGGGGSQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 49 - protein, 588 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSGGGGSGGGGSGGGGSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 336 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGGSGGGGSGGGGSDIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 582 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSQVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 337 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGGSGGGGSGGGGSDIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 587 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSGGGGSGGGGSGGGGSQVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 577 aa, synthetic construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGGSGGGGSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 577 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 56 - protein, 576 aa, synthetic construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGGSGGGGSQVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 576 aa, synthetic construct
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 58 - protein, 330 aa, synthetic construct [sequence omitted]

SEQ ID NO: 59 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 62 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 65 - protein, 232 aa, synthetic construct; identical to SEQ ID NO: 62

SEQ ID NO: 66 - protein, 232 aa, synthetic construct; identical to SEQ ID NO: 63

SEQ ID NO: 67 - protein, 232 aa, synthetic construct; identical to SEQ ID NO: 64

SEQ ID NO: 68 - protein, 107 aa, synthetic construct [sequence omitted]

SEQ ID NO: 69 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 70 - protein, 15 aa, synthetic construct
EPKSCDKTHTCPPCP

SEQ ID NO: 71 - protein, 5 aa, synthetic construct
  REPEAT 1..5: note = (GGGGS)n, n=1, 2, 3, 4, or 5
GGGGS

SEQ ID NO: 72 - protein, 15 aa, synthetic construct
GGGGSGGGGSGGGGS

SEQ ID NO: 73 - protein, 5 aa, synthetic construct
  REPEAT 1..5: note = (EAAAK)n, n=1, 2, 3, 4, or 5
EAAAK

SEQ ID NO: 74 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 75 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 76 - protein, 22 aa, Homo sapiens
GSGATNFSLLKQAGDVEENPGP

SEQ ID NO: 77 - protein, 23 aa, Homo sapiens
GSGQCTNYALLKLAGDVESNPGP

SEQ ID NO: 78 - protein, 25 aa, Homo sapiens
GSGVKQTLNFDLLKLAGDVESNPGP

SEQ ID NO: 79 - protein, 21 aa, Homo sapiens
GSGEGRGSLLTCGDVEENPGP

SEQ ID NO: 80 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 81 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 82 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 84 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 85 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 86 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 87 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 88 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 12 aa, Homo sapiens
ERKCCVECPPCP

SEQ ID NO: 90 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 91 - protein, 12 aa, Homo sapiens
ESKYGPPCPSCP

SEQ ID NO: 92 - protein, 714 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGCGTKLTVLGGGGSGGGGSGGGGSEVQLVQSGAEVKKPGESLKISCKGSGYSFSNYWIGWVRQMPGKCLEWMGIIDPSNSYTRYSPSFQGQVTISADKSISTAYLQWSSLKASDTAMYYCARWYYKPFDVWGQGTLVTVSS

SEQ ID NO: 93 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGCGTKLTVL

SEQ ID NO: 94 - protein, 117 aa, synthetic construct
EVQLVQSGAEVKKPGESLKISCKGSGYSFSNYWIGWVRQMPGKCLEWMGIIDPSNSYTRYSPSFQGQVTISADKSISTAYLQWSSLKASDTAMYYCARWYYKPFDVWGQGTLVTVSS

(19) United States
(12) Patent Application Publication
(10) Pub. No.: US 2026/0209331 A1
(43) Pub. Date: Jul. 23, 2026

(54) ANTIBODIES TARGETING TNF ALPHA AND IL-23 AND USES THEREOF

(71) Applicant: INMAGENE PTE. LTD., Singapore (SG)

(72) Inventors: Run Lei, Shanghai (CN); Pengcheng Fan, Hangzhou, Zhejiang (CN); Chongtian Guo, Shanghai (CN); Yu Wang, Hangzhou, Zhejiang (CN); Jonathan Jian Wang, Shanghai (CN); Xiaotong Wu, Shanghai (CN)

(21) Appl. No.: 19/135,817

(22) PCT Filed: Sep. 20, 2023
(86) PCT No.: PCT/CN2023/120148

§ 371 Date: Jun. 5, 2025

(30) Foreign Application Priority Data
Sep. 21, 2022 (WO) PCT/CN2022/120342

(51) Int. Cl. C07K 16/24; A61K 39/00; A61P 37/06

(52) U.S. Cl. CPC C07K 16/241; A61P 37/06; C07K 16/244; A61K 2039/505; C07K 2317/31; C07K 2317/569; C07K 2317/92

(57) ABSTRACT

Provided are bispecific antibodies targeting both human TNFα and P19 subunit of human IL-23. Polynucleotides encoding the bispecific antibodies, pharmaceutical compositions comprising the bispecific antibodies, and methods of producing the bispecific antibodies are also provided herein. Medical uses of the bispecific antibodies described herein are also provided.

### FIELD

The present disclosure relates to molecular biology and immunology. Provided herein include bispecific antibodies targeting both human TNF alpha (TNFα) and the P19 subunit of human IL-23 (IL23p19) as well as uses thereof in treating autoimmune diseases and inflammatory diseases.

### BACKGROUND

Autoimmune diseases and inflammatory diseases, which can arise from the body's production of an immune response against its own tissue, are often chronic and can be debilitating and even life-threatening. TNFα is a potent inducer of inflammatory response, and Interleukin 23 (IL-23) is an upstream regulator in tissue inflammation. Both TNFα and IL-23 are clinically validated targets. However, there is still a great unmet need for additional therapeutic options for patients who show inadequate responses to available therapeutics targeting either TNFα or IL-23. The bispecific antibodies that target both human TNFα and human IL23p19 and related methods provided herein meet these needs and provide relative advantages.

### SUMMARY

Provided herein are bispecific antibodies that specifically bind to human TNF alpha (TNFα) and to the P19 subunit of human IL-23 (IL23p19) and related pharmaceutical compositions, polynucleotides, vectors, and cells. Methods of producing these bispecific antibodies and using these bispecific antibodies are also provided herein. Some exemplary embodiments are provided below.

Embodiment 1: Bispecific antibodies that specifically bind to human TNFα and to human IL23p19, comprising: (1) a first light chain (LC1) comprising a first light chain variable domain (VL1) and a first heavy chain constant domain 1 (CH1); (2) a first heavy chain (HC1) comprising a first heavy chain variable domain (VH1), a first light chain constant region (CL), and a Knob-Fc region; (3) a second light chain (LC2) comprising a second light chain variable domain (VL2) and a second CL region; and (4) a second heavy chain (HC2) comprising a second heavy chain variable domain (VH2), a second CH1 domain, and a Hole-Fc region; wherein (i) the VL1/VH1 pair and the VL2/VH2 pair specifically bind to human TNFα and human IL23p19, respectively, or to human IL23p19 and human TNFα, respectively; and (ii) the Knob-Fc region is a human IgG Fc region variant having a T366W substitution (numbered according to the EU Index); and the Hole-Fc region is a human IgG Fc region variant having a Y407V substitution (numbered according to the EU Index).

Embodiment 2: The bispecific antibodies of Embodiment 1, wherein (1) the first CL region is kappa CL (Cκ; SEQ ID NO: 68) or lambda CL (Cλ, SEQ ID NO: 69); and the second CL region is Cκ (SEQ ID NO: 68) or Cλ (SEQ ID NO: 69); (2) the first CH1 domain and the second CH1 domain are both human IgG1 CH1 domain (SEQ ID NO: 61); or (3) the Knob-Fc region has the amino acid sequence of SEQ ID NO: 66; and the Hole-Fc region has the amino acid sequence of SEQ ID NO: 67; or any combination of (1)-(3).

Embodiment 3: The bispecific antibodies of Embodiment 1 or 2, wherein the VL1/VH1 pair specifically binds to human TNFα and the VL2/VH2 pair specifically binds to human IL23p19.

Embodiment 4: The bispecific antibodies of Embodiment 3, wherein the VL1 and VH1 have the amino acid sequences of (1) SEQ ID NOs: 1 and 2, respectively; or (2) SEQ ID NOs: 5 and 6, respectively; or wherein the VL2 and VH2 have the amino acid sequences of (1) SEQ ID NOs: 10 and 11, respectively; or (2) SEQ ID NOs: 14 and 15, respectively.

Embodiment 5: The bispecific antibodies of Embodiment 3, wherein the VL1, VH1, VL2 and VH2 have the amino acid sequences of (1) SEQ ID NOs: 1, 2, 10, and 11, respectively; (2) SEQ ID NOs: 1, 2, 14, and 15, respectively; (3) SEQ ID NOs: 5, 6, 10, and 11, respectively; or (4) SEQ ID NOs: 5, 6, 14, and 15, respectively.

Embodiment 6: The bispecific antibodies of Embodiment 3, wherein LC1, HC1, LC2, and HC2 each has at least 90%, at least 95%, at least 98%, at least 99%, or 100% sequence identity to the amino acid sequences of (1) SEQ ID NOs: 24, 25, 12, and 26, respectively; (2) SEQ ID NOs: 27, 28, 12, and 26, respectively; (3) SEQ ID NOs: 24, 25, 16, and 29, respectively; or (4) SEQ ID NOs: 27, 28, 16, and 29, respectively.

Embodiment 7: The bispecific antibodies of Embodiment 1 or 2, wherein the VL1/VH1 pair specifically binds to human IL23p19 and the VL2/VH2 pair specifically binds to human TNFα.

Embodiment 8: The bispecific antibodies of Embodiment 7, wherein VL1 and VH1 have the amino acid sequences of (1) SEQ ID NOs: 10 and 11, respectively; or (2) SEQ ID NOs: 14 and 15, respectively; or wherein VL2 and VH2 have the amino acid sequences of (1) SEQ ID NOs: 1 and 2, respectively; or (2) SEQ ID NOs: 5 and 6, respectively.

Embodiment 9: The bispecific antibodies of Embodiment 7, wherein VL1, VH1, VL2 and VH2 have the amino acid sequences of (1) SEQ ID NOs: 10, 11, 1, and 2, respectively; (2) SEQ ID NOs: 10, 11, 5, and 6, respectively; (3) SEQ ID NOs: 14, 15, 1, and 2, respectively; or (4) SEQ ID NOs: 14, 15, 5, and 6, respectively.

Embodiment 10: The bispecific antibodies of Embodiment 7, wherein LC1, HC1, LC2, and HC2 each has at least 90%, at least 95%, at least 98%, at least 99%, or 100% sequence identity to the amino acid sequences of (1) SEQ ID NOs: 18, 19, 3, and 20, respectively; (2) SEQ ID NOs: 21, 22, 3, and 20 respectively; (3) SEQ ID NOs: 18, 19, 7 and 23, respectively; or (4) SEQ ID NOs: 21, 22, 7, and 23, respectively.

Embodiment 11: Bispecific antibodies that specifically bind to human TNFα and to human IL23p19, comprising: (1) a light chain (LC) comprising a first light chain variable domain (VL1) and a CL region; and (2) a heavy chain (HC) comprising a first heavy chain variable domain (VH1), a heavy chain constant region (CH), a second light chain variable domain (VL2); and a second heavy chain variable domain (VH2); wherein the VL1/VH1 pair and VL2/VH2 pair specifically bind to human TNFα and human IL23p19, respectively, or to human IL23p19 and human TNFα, respectively.

Embodiment 12: The bispecific antibodies of Embodiment 11, wherein (1) the CL region is Cκ (SEQ ID NO: 68) or Cλ (SEQ ID NO: 69); or (2) the CH region is human IgG1 CH region (SEQ ID NO: 58); or both (1) and (2).

Embodiment 13: The bispecific antibodies of Embodiment 11 or 12, wherein the VL1/VH1 pair specifically binds to human TNFα and the VL2/VH2 pair specifically binds to human IL23p19.

Embodiment 14: The bispecific antibodies of Embodiment 13, wherein the VL1 and VH1 have the amino acid sequences of (1) SEQ ID NOs: 1 and 2, respectively; or (2) SEQ ID NOs: 5 and 6, respectively; or wherein the VL2 and VH2 have the amino acid sequences of (1) SEQ ID NOs: 10 and 11, respectively; or (2) SEQ ID NOs: 14 and 15, respectively; or (3) SEQ ID NOs: 93 and 94, respectively.

Embodiment 15: The bispecific antibodies of Embodiment 13, wherein the VL1, VH1, VL2 and VH2 have the amino acid sequences of (1) SEQ ID NOs: 1, 2, 10, and 11, respectively; (2) SEQ ID NOs: 1, 2, 14, and 15, respectively; (3) SEQ ID NOs: 1, 2, 93, and 94, respectively; (4) SEQ ID NOs: 5, 6, 10, and 11, respectively; or (5) SEQ ID NOs: 5, 6, 14, and 15, respectively; (6) SEQ ID NOs: 5, 6, 93, and 94, respectively.

Embodiment 16: The bispecific antibodies of Embodiment 13, wherein the LC and HC each has at least 90%, at least 95%, at least 98%, at least 99%, or 100% sequence identity to the amino acid sequences of (1) SEQ ID NOs: 3 and 30, respectively; (2) SEQ ID NOs: 3 and 31 respectively; (3) SEQ ID NOs: 7 and 32, respectively; (4) SEQ ID NOs: 7 and 33, respectively; or (5) SEQ ID NOs: 7 and 92, respectively.

Embodiment 17: The bispecific antibodies of Embodiment 11 or 12, wherein the VL1/VH1 pair specifically binds to human IL23p19 and the VL2/VH2 pair specifically binds to human TNFα.

Embodiment 18: The bispecific antibodies of Embodiment 17, wherein the VL1 and VH1 have the amino acid sequences of (1) SEQ ID NOs: 10 and 11, respectively; or (2) SEQ ID NOs: 14 and 15, respectively; or wherein VL2 and VH2 have the amino acid sequences of (1) SEQ ID NOs: 1 and 2, respectively; or (2) SEQ ID NOs: 5 and 6, respectively.

Embodiment 19: The bispecific antibodies of Embodiment 17, wherein the VL1, VH1, VL2 and VH2 have the amino acid sequences of (1) SEQ ID NOs: 10, 11, 1, and 2, respectively; (2) SEQ ID NOs: 10, 11, 5, and 6, respectively; (3) SEQ ID NOs: 14, 15, 1, and 2, respectively; or (4) SEQ ID NOs: 14, 15, 5, and 6, respectively.

Embodiment 20: The bispecific antibodies of Embodiment 17, wherein the LC and HC each has at least 90%, at least 95%, at least 98%, at least 99%, or 100% sequence identity to the amino acid sequences of (1) SEQ ID NOs: 12 and 34, respectively; (2) SEQ ID NOs: 12 and 35, respectively; (3) SEQ ID NOs: 16 and 36, respectively; or (4) SEQ ID NOs: 16 and 37, respectively.

Embodiment 21: The bispecific antibodies that specifically bind to human TNFα and to human IL23p19, comprising: (1) a light chain (LC) comprising a first light chain variable domain (VL1), a second light chain variable domain (VL2), and a CL region; and (2) a heavy chain (HC) comprising a first heavy chain variable domain (VH1), a second heavy chain variable domain (VH2), and a CH region; wherein the VL1/VH1 pair and VL2/VH2 pair specifically bind to human TNFα and human IL23p19, respectively, or to human IL23p19 and human TNFα, respectively.

Embodiment 22: The bispecific antibodies of Embodiment 21, wherein (1) the CL region is Cκ (SEQ ID NO: 68) or Cλ (SEQ ID NO: 69); or (2) the CH region is human IgG1 CH region (SEQ ID NO: 58); or both (1) and (2).

Embodiment 23: The bispecific antibodies of Embodiment 21 or 22, wherein the VL1/VH1 pair specifically binds to human TNFα and the VL2/VH2 pair specifically binds to human IL23p19.

Embodiment 24: The bispecific antibodies of Embodiment 23, wherein the VL1 and VH1 have the amino acid sequences of (1) SEQ ID NOs: 1 and 2, respectively; or (2) SEQ ID NOs: 5 and 6, respectively; or wherein VL2 and VH2 have the amino acid sequences of (1) SEQ ID NOs: 10 and 11, respectively; or (2) SEQ ID NOs: 14 and 15, respectively.

Embodiment 25: The bispecific antibodies of Embodiment 23, wherein the VL1, VH1, VL2 and VH2 have the amino acid sequences of (1) SEQ ID NOs: 1, 2, 10, and 11, respectively; (2) SEQ ID NOs: 1, 2, 14, and 15, respectively; (3) SEQ ID NOs: 5, 6, 10, and 11, respectively; or (4) SEQ ID NOs: 5, 6, 14, and 15, respectively.

Embodiment 26: The bispecific antibodies of Embodiment 23, wherein the LC and HC each has at least 90%, at least 95%, at least 98%, at least 99%, or 100% sequence identity to the amino acid sequences of (1) SEQ ID NOs: 46 and 47, respectively; (2) SEQ ID NOs: 48 and 49 respectively; (3) SEQ ID NOs: 50 and 51, respectively; or (4) SEQ ID NOs: 52 and 53, respectively.

Embodiment 27: The bispecific antibodies of Embodiment 21 or 22, wherein the VL1/VH1 pair specifically binds to human IL23p19 and the VL2/VH2 pair specifically binds to human TNFα.

Embodiment 28: The bispecific antibodies of Embodiment 27, wherein the VL1 and VH1 have the amino acid sequences of (1) SEQ ID NOs: 10 and 11, respectively; or (2) SEQ ID NOs: 14 and 15, respectively; or wherein VL2 and VH2 have the amino acid sequences of (1) SEQ ID NOs: 1 and 2, respectively; or (2) SEQ ID NOs: 5 and 6, respectively.

Embodiment 29: The bispecific antibodies of Embodiment 27, wherein the VL1, VH1, VL2 and VH2 have the amino acid sequences of (1) SEQ ID NOs: 10, 11, 1, and 2, respectively; (2) SEQ ID NOs: 10, 11, 5, and 6, respectively; (3) SEQ ID NOs: 14, 15, 1, and 2, respectively, or (4) SEQ ID NOs: 14, 15, 5, and 6, respectively.

Embodiment 30: The bispecific antibodies of Embodiment 27, wherein the LC and HC each has at least 90%, at least 95%, at least 98%, at least 99%, or 100% sequence identity to the amino acid sequences of (1) SEQ ID NOs: 38 and 39, respectively; (2) SEQ ID NOs: 40 and 41, respectively; (3) SEQ ID NOs: 42 and 43, respectively; or (4) SEQ ID NOs: 44 and 45, respectively.

Embodiment 31: Bispecific antibodies that specifically bind to human TNFα and to human IL23p19, comprising: (1) a light chain (LC) comprising a light chain variable domain (VL) and a CL region; and (2) a heavy chain (HC) comprising a heavy chain variable domain (VH) and a CH region; wherein (1) the VL/VH pair specifically binds to human IL23p19; and (2) the HC further comprises a single heavy chain variable domain antibody (VHH) that specifically binds to human TNFα.

Embodiment 32: The bispecific antibodies of Embodiment 31, wherein (1) the CL region is Cκ (SEQ ID NO: 68) or Cλ (SEQ ID NO: 69); or (2) the CH region is human IgG1 CH region (SEQ ID NO: 58); or both (1) and (2).

Embodiment 33: The bispecific antibodies of Embodiment 31 or 32, wherein the VL and VH have the amino acid sequences of (1) SEQ ID NOs: 10 and 11, respectively; or (2) SEQ ID NOs: 14 and 15, respectively.

Embodiment 34: The bispecific antibodies of any one of Embodiments 31 to 33, wherein the VHH has the amino acid sequence of SEQ ID NO: 9.

Embodiment 35: The bispecific antibodies of any one of Embodiments 31 to 34, wherein the VHH is linked to the N-terminus of the VH.

Embodiment 36: The bispecific antibodies of Embodiment 35, wherein the LC and HC each has at least 90%, at least 95%, at least 98%, at least 99%, or 100% sequence identity to the amino acid sequences of (1) SEQ ID NOs: 12 and 54, respectively; or (2) SEQ ID NOs: 16 and 56 respectively.

Embodiment 37: The bispecific antibodies of any one of Embodiments 31 to 34, wherein the VHH is linked to the C-terminus of the Fc domain.

Embodiment 38: The bispecific antibodies of Embodiment 37, wherein the LC and the HC each has at least 90%, at least 95%, at least 98%, at least 99%, or 100% sequence identity to the amino acid sequences of (1) SEQ ID NOs: 12 and 55, respectively; or (2) SEQ ID NOs: 16 and 57, respectively.

Embodiment 39: Pharmaceutical compositions comprising the bispecific antibodies of any one of Embodiments 1 to 38 and a pharmaceutically acceptable carrier.

Embodiment 40: Methods of reducing TNFα and/or IL23p19-associated autoimmunity or inflammation in a subject in need thereof comprising administering to the subject a therapeutically effective amount of the bispecific antibodies of any one of Embodiments 1 to 38.

Embodiment 41: The methods of Embodiment 45, wherein the subject has an autoimmune disease or an inflammatory disease.

Embodiment 42: Methods of treating an autoimmune disease in a subject in need thereof comprising administering to the subject a therapeutically effective amount of the bispecific antibodies of any one of Embodiments 1 to 38.

Embodiment 43: Methods of treating an inflammatory disease in a subject in need thereof comprising administering to the subject a therapeutically effective amount of the bispecific antibodies of any one of Embodiments 1 to 38.

Embodiment 44: The methods of any one of Embodiments 45 to 48, wherein the subject is a human.

Embodiment 45: Uses of the bispecific antibodies of any one of Embodiments 1 to 38 as a medicament.

Embodiment 46: Uses of the bispecific antibodies of any one of Embodiments 1 to 38 in treating an autoimmune disease.

Embodiment 47: Uses of the bispecific antibodies of any one of Embodiments 1 to 38 in treating an inflammatory disease.

Embodiment 48: Uses of the bispecific antibodies of any one of Embodiments 1 to 38 for the preparation of a medicament for treating an autoimmune disease.

Embodiment 49: Uses of the bispecific antibodies of any one of Embodiments 1 to 38 for the preparation of a medicament for treating an inflammatory disease.

Embodiment 50: Polynucleotides encoding the LC1, the LC2, the HC1, the HC2, or any combination thereof, of the bispecific antibodies of any one of Embodiments 1 to 10.

Embodiment 51: Polynucleotides encoding the LC, the HC, or both the LC and HC of the bispecific antibodies of any one of Embodiments 11 to 38.

Embodiment 52: Vectors comprising the polynucleotides of Embodiment 50 or 51.

Embodiment 53: Cells comprising (a) the polynucleotides of Embodiment 50 that encode the LC1, LC2, HC1, and HC2, or (b) a plurality of the polynucleotides of Embodiment 50 that collectively encode the LC1, LC2, HC1, and HC2.

Embodiment 54: Cells comprising (a) the polynucleotides of Embodiment 51 that encode both the LC and HC, or (b) a first polynucleotide of Embodiment 51 that encodes the LC and a second polynucleotide of Embodiment 51 that encodes the HC.

Embodiment 55: Methods of producing bispecific antibodies that specifically bind to human TNFα and to human IL23p19 by expressing the polynucleotides or the plurality of polynucleotides in the cell of Embodiment 53 or 54.

### BRIEF DESCRIPTION OF DRAWINGS

FIGS. 1A-1D provide diagrams illustrating the four different types of anti-TNFα/IL23p19 bispecific antibodies disclosed herein. (N) means the N-terminus; (C) means the C-terminus.

FIG. 1A illustrates the “CrossMab-KIH” structure, which includes four distinct polypeptides, a first light chain (LC1), a first heavy chain (HC1), a second light chain (LC2), and a second heavy chain (HC2), with the configurations shown below:

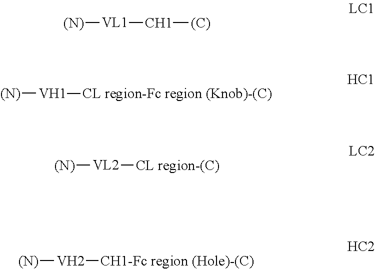

FIG. 1B illustrates the “IgG-ScFv” structure, which includes two identical pairs of light chains (LC) and heavy chains (HC), with the configurations below:

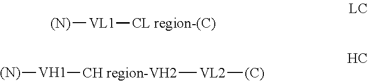

FIG. 1C illustrates the “DVD-Ig” structure, which includes two identical pairs of light chains (LC) and heavy chains (HC), with the configurations below:

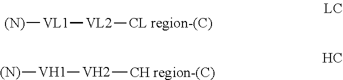

FIG. 1D illustrates the “SMAB-VHH” structure, which includes two identical pairs of light chains (LC) and heavy chains (HC), with the configurations below:

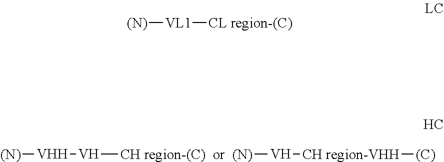

FIG. 2 shows a chart summarizing the binding affinities of exemplary anti-TNFα/IL23p19 bispecific antibodies to TNFα and IL23p19, respectively.

FIG. 3 provides a chart demonstrating the simultaneous binding of the exemplary anti-TNFα/IL23p19 bispecific antibodies to both antigens, namely, human TNFα and IL23p19.

FIGS. 4A-4C provide graphs showing effective inhibition of TNFα-induced NFκB signaling by exemplary anti-TNFα/IL23p19 bispecific antibodies.

FIGS. 5A-5C provide graphs showing effective inhibition of IL-23 induced STAT3 phosphorylation by exemplary anti-TNFα/IL23p19 bispecific antibodies.

FIG. 6 provides graphs showing effective inhibition of TNFα-induced U937 cell apoptosis by exemplary anti-TNFα/IL23p19 bispecific antibodies.

FIG. 7 provides graphs showing effective inhibition of TNFα-induced L929 cell cytotoxicity by exemplary anti-TNFα/IL23p19 bispecific antibodies.

FIG. 8 provides graphs showing effective inhibition of TNFα combination with IL-23-induced human IL-17 cytokine production in human PBMC by exemplary anti-TNFα/IL23p19 bispecific antibodies.

FIG. 9 provides graphs showing effective inhibition of human TNFα-induced mIL-6 production in mice by exemplary anti-TNFα/IL23p19 bispecific antibodies.

FIG. 10 provides graphs showing effective inhibition of human IL-23-induced ear hyperplasia in mice by exemplary anti-TNFα/IL23p19 bispecific antibodies, with ear thickness, clinical PASI score and ear histopathology score evaluated.

### DETAILED DESCRIPTION

The present disclosure provides novel bispecific antibodies that specifically bind to both human TNF alpha (TNFα) and to the P19 subunit of human IL-23 (IL23p19). Pharmaceutical compositions comprising a therapeutically effective amount of such antibodies are also disclosed herein. Also disclosed herein are uses of such antibodies and pharmaceutical compositions for reducing autoimmunity, and for treating autoimmune diseases and inflammatory diseases.

Before the present disclosure is further described, it is to be understood that the disclosure is not limited to the particular embodiments set forth herein, and it is also to be understood that the terminology used herein is for the purpose of describing particular embodiments, and is not intended to be limiting.

### 1. Definitions

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The term “a” or “an” entity refers to one or more of that entity; for example, “an antibody,” is understood to represent one or more antibodies.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “polypeptide,” “peptide,” “protein,” “polypeptide chain,” “peptide chain,” and their grammatical equivalents as used interchangeably herein refer to polymers of amino acids of any length, which can be linear or branched. It can include unnatural or modified amino acids or be interrupted by non-amino acids. A polypeptide, peptide, polypeptide chain, peptide chain, or protein can also be modified with, for example, disulfide bond formation, glycosylation, lipidation, acetylation, phosphorylation, or any other manipulation or modification.

The terms “polynucleotide,” “nucleic acid,” and their grammatical equivalents as used interchangeably herein mean polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase.

The term “variant” as used herein in relation to a protein or a polypeptide with particular sequence features (the “reference protein” or “reference polypeptide”) refers to a different protein or polypeptide having one or more (such as, for example, about 1 to about 25, about 1 to about 20, about 1 to about 15, about 1 to about 10, or about 1 to about 5) amino acid substitutions, deletions, and/or additions as compared to the reference protein or reference polypeptide. The changes to an amino acid sequence can be amino acid substitutions. The changes to an amino acid sequence can be conservative amino acid substitutions. A functional fragment or a functional variant of a protein or polypeptide maintains the basic structural and functional properties of the reference protein or polypeptide.

The term “specifically binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that specifically binds a target molecule (e.g., antigen) can be identified, for example, by immunoassays, ELISAs, Bio-Layer Interferometry (“BLI”), SPR (e.g., Biacore), or other techniques known to those of skill in the art. Typically, a specific reaction will be at least twice background signal or noise and can be more than 10 times background. See, e.g., Paul, ed., 1989, FUNDAMENTAL IMMUNOLOGY SECOND EDITION, Raven Press, New York at pages 332-336 for a discussion regarding antibody specificity. A binding moiety that specifically binds a target molecule can bind the target molecule at a higher affinity than its affinity for a different molecule. In some embodiments, a binding moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a K_{D} of about 0.1 mM or less. In some embodiments, “specifically binds” means that a polypeptide or molecule binds a target with a K_{D} of at about 10 μM or less or about 1 μM or less. In some embodiments, “specifically binds” means that a polypeptide or molecule binds a target with a K_{D} of at about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety (e.g., antibody) that specifically binds a first target may or may not specifically bind a second target. As such, “specific binding” does not necessarily require (although it can include) exclusive binding, i.e., binding to a single target. Thus, a binding moiety (e.g., antibody) can, in some embodiments, specifically bind more than one target. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities.

The term “binding affinity” as used herein generally refers to the strength of the sum total of noncovalent interactions between a binding moiety and a target molecule (e.g., antigen). The binding of a binding moiety and a target molecule is a reversible process, and the affinity of the binding is typically reported as an equilibrium dissociation constant (K_{D}). K_{D} is the ratio of a dissociation rate (k_{off} or k_{d}) to the association rate (kₒₙ or kₐ). The lower the K_{D} of a binding pair, the higher the affinity. A variety of methods of measuring binding affinity are known in the art, any of which can be used for purposes of the present disclosure. Specific illustrative embodiments include the following. In some embodiments, the “K_{D}” or “K_{D} value” can be measured by assays known in the art, for example by a binding assay. The K_{D} may be measured in a radiolabeled antigen binding assay (RIA) (Chen, et al., (1999) J. Mol Biol 293:865-881). The K_{D} or K_{D} value can also be measured by using biolayer interferometry (BLI) using, for example, the Gator system (Probe Life), or the Octet-96 system (Sartorius AG). The K_{D} or K_{D} value can also be measured by using surface plasmon resonance assays (SPR) by Biacore, using, for example, a BIAcore™-2000 or a BIAcore™-3000 BIAcore, Inc., Piscataway, NJ).

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NOS. It is understood that one skilled in the art can readily identify homologous sequences by reference to sequence sources, including but not limited to Uniprot (https://www.uniprot.org/), GenBank (ncbi.nlm.nih.gov/genbank/) and EMBL (embl.org/).

### 2. Bispecific Antibodies Targeting TNFα and IL23p19

Provided herein are bispecific antibodies that specifically bind to both human TNFα and human IL23p19. In some embodiments, the bispecific antibodies provided herein are monoclonal antibodies. In some embodiments, the bispecific antibodies provided herein are isolated. In some embodiments, the bispecific antibodies provided herein are substantially pure. 2.1 General

Tumor Necrosis Factor alpha (TNFα) is a pleiotropic homotrimeric cytokine. TNFα is primarily secreted by monocytes, macrophages, lymphocytes, endothelial cells and fibroblasts. TNFα binds two distinct receptors: TNFRI, expressed on nearly all cell types and TNFRII, with limited expression on immune cells (CD4+ T cells, NK cells). TNFα is expressed in both a soluble and transmembrane form (the membrane-bound precursor form can be proteolytically cleaved into a soluble homotrimer by metalloproteinase TNF alpha converting enzyme (TACE)). Both membrane-bound and soluble forms of the cytokine are biologically active. TNFα is a potent inducer of inflammatory response. It promotes the production of pro-inflammatory cytokines and chemokines, increases the recruitment and infiltration of leukocyte, and activates both innate and adaptive immunity. As such TNFα can be important in systemic inflammation, specifically in acute phase inflammatory reactions. Excess amounts of TNFα have been associated with various forms of autoimmune diseases. Silva et al., Immunotherapy (2010) 2(6), 817-833; Salomon, Nat. Rev. Rheumatol. 17(8):487-504 (2021). An exemplary amino acid sequence of human TNFα is provided below:

(SEQ ID NO: 74; Uniprot ID: P01375-1)
MSTESMIRDVELAEEALPKKTGGPQGSRRCLFLSLFSFLIVAGAT
TLFCLLHFGVIGPQREEFPRDLSLISPLAQAVRSSSRTPSDKPVA
HVVANPQAEGQLQWLNRRANALLANGVELRDNQLVVPSEGLYLIY
SQVLFKGQGCPSTHVLLTHTISRIAVSYQTKVNLLSAIKSPCQRE
TPEGAEAKPWYEPIYLGGVFQLEKGDRLSAEINRPDYLDFAESGQ
VYFGIIAL

Interleukin 23 (IL23) is a heterodimeric cytokine consisting of two subunits, p40 (shared with IL12) and p19 (unique to IL23). The p19 subunit is also referred to as IL23A. IL23 binds to a cell surface receptor composed of the IL12 receptor β1 subunit and a unique IL23 receptor subunit. Expression of the IL23R is restricted to specific populations of immune cells and is found primarily on subsets of T cells (αβ and γδ TCR+) and NK cells. IL23 is an upstream regulator in tissue inflammation for IL-6, IL-17, GM-CSF and IL-22. IL23 signals downstream via TYK2/JAK2 mediated STAT3 phosphorylation. IL23 promotes the differentiation of development of naïve CD4+ T cell to pathogenic Th17 cells, and stimulates iNKT cells, γδ T cells and ILC3s, to produce IL17 family cytokines. IL23 also promotes osteoclastogenesis and bone resorption. IL23 promotes the activation of a range of inflammatory cells involved in the induction of chronic inflammation, regulates both memory/pathogenic T-cell inflammatory response as well as innate lymphoid cell inflammatory activity. As such, elevated IL23 production has been implicated as being a major factor in inflammatory diseases and autoimmune diseases. Moschen et al., Nat. Rev. Gastroenterol. Hepatol. 16(3):185-196 (2019); Schmitt et al., Front Immunol. 2021; 12:622934; Silvagni et al., Front Pharmacol., 2021; 12:672515. An exemplary amino acid sequence of the p19 subunit human IL23 is provided below:

(SEQ ID NO: 75; Uniprot ID: Q9NPF7)
MLGSRAVMLLLLLPWTAQGRAVPGGSSPAWTQCQQLSQKLCTLAW
SAHPLVGHMDLREEGDEETTNDVPHIQCGDGCDPQGLRDNSQFCL
QRTHQGLIFYEKLLGSDIFTGEPSLLPDSPVGQLHASLLGLSQLL
QPEGHHWETQQIPSLSPSQPWQRLLLRFKILRSLQAFVAVAARVF
AHGAATLSP

As used herein and understood in the art, an “antibody” is an immunoglobulin molecule that recognizes and specifically binds a target, such as a protein, polypeptide, peptide, carbohydrate, polynucleotide, lipid, or a combination of any of the foregoing, through at least one antigen-binding site which is typically within the variable region of the immunoglobulin molecule. A “bispecific” antibody is an artificial hybrid antibody having two different antigen binding sites, which recognize and specifically bind two different target antigens.

The term “antibody” is used herein in its broadest sense to encompass antibodies of different types and structures, including polyclonal antibodies, monoclonal antibodies, multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. Unless expressly indicated otherwise, the term “antibody” as used herein include “antigen-binding fragment” of intact antibodies. The term “antigen-binding fragment” as used herein refers to a portion or fragment of an intact antibody that is the antigenic determining variable region of an intact antibody. Examples of antigen-binding fragments include, but are not limited to, Fab, Fab′, F(ab′)2, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH), and bispecific or multispecific antibodies formed from antibody fragments.

The structure of immunoglobulins has been well characterized (see, e.g., FUNDAMENTAL IMMUNOLOGY Ch. 7 (Paul, W., ed., 2nd ed. Raven Press, N.Y. (1989)). Typically, immunoglobulins comprise two pairs of polypeptide chains, one pair of light (L; low molecular weight) chains and one pair of heavy (H; high molecular weight) chains, all four inter-connected by disulfide bonds.

Each light chain of an immunoglobulin typically includes a light chain variable region (“VL region”) and a light chain constant region (“CL region”). There are two distinct types of light chains, referred to as kappa (κ) of lambda (λ) based on the amino acid sequence of the CL region. The amino acid sequences of the CL regions are well known in the art.

Each heavy chain typically includes a heavy chain variable region (a “VH region”) and a heavy chain constant region (a “CH region”). The VH region can be one of five distinct types, referred to as alpha (α), delta (δ), epsilon (ε), gamma (γ) and mu (μ), based on the amino acid sequence. When combined with a light chain, these distinct types of heavy chains give rise to five well known classes of antibodies, IgA, IgD, IgE, IgG and IgM, respectively. There are four subclasses of IgG, namely, IgG1, IgG2, IgG3 and IgG4. The amino acid sequences of the CH regions of different classes of antibodies are well known in the art.

The CH region of immunoglobulins comprise more than one domain. For example, the CH region of an IgG antibody is comprised of three domains, heavy chain constant domain 1 (CH1), heavy chain constant domain 2 (CH2), and heavy chain constant domain 3 (CH3). The highly flexible region between the CH1 and CH2 domains is referred to as the “hinge region.” Disulfide bonds in the hinge region are part of the interactions between two heavy chains in an immunoglobulin. The “Fc region” refers to the C-terminal region of an immunoglobulin heavy chain that contains at least a portion of the constant region. In IgG, IgA and IgD isotypes, the Fc region is comprised of the CH2 domain and the CH3 domain; IgM and IgE Fc regions contain three heavy chain constant domains (CH domains 2-4). The amino acid sequences of the Fc region of human IgG, IgA, IgD, IgM and IgE, and subtypes IgG1, IgG2, IgG3, and IgG4 are known to those of ordinary skill in the art. Although the boundaries of the Fc region of an IgG heavy chain might vary slightly, the human IgG heavy chain Fc region is usually defined to extend from the hinge region to the carboxyl-terminus of the heavy chain.

The term “Fc region” as used herein includes native sequence Fc regions and variant Fc regions. In some embodiments, the Fe domains of the two heavy chains of a bispecific antibody provided herein can comprise paired modifications that promote their association with each other, instead of forming homodimers.

Unless otherwise stated or contradicted by context, reference to amino acid positions in the constant regions is according to the EU-numbering (Edelman et al., PNAS. 1969; 63:78-85, Kabat et al., SEQUENCES OF PROTEINS OF IMMUNOLOGICAL INTEREST, Fifth Edition. 1991 NIH Publication No. 91-3242). A list of exemplary amino acid sequences for constant domains/regions of the human IgG antibodies is provided below as Table 1.

TABLE 1
Amino acid sequences of human IgG constant regions/domains.

| Domains/regions | Sequences |
|---|---|
| Human IgG CK | RTVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 68) |
| Human IgG CA | GQPKAAPSVTLFPPSSEELQANKATLVCLISDFYPGAVTVAWKADSSPVKAGVE |
| | TTTPSKQSNNKYAASSYLSLTPEQWKSHRSYSCQVTHEGSTVEKTVAPTECS |
| | (SEQ ID NO: 69) |
| Human IgG1 CH | ASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLS |
| | LSPGK (SEQ ID NO: 58) |
| Human IgG2 CH | ASTKGPSVFPLAPCSRSTSESTAALGCLVKDYFPEPVTVSWNSGALTSGVHTFPA |
| | VLQSSGLYSLSSVVTVPSSNFGTQTYTCNVDHKPSNTKVDKTVERKCCVECPPC |
| | PAPPVAGPSVFLFPPKPKDTLMISRTPEVTCVVVDVSHEDPEVQFNWYVDGVEV |
| | HNAKTKPREEQFNSTFRVVSVLTVVHQDWLNGKEYKCKVSNKGLPAPIEKTISK |
| | TKGQPREPQVYTLPPSREEMTKNQVSLTCLVKGFYPSDISVEWESNGQPENNYK |
| | TTPPMLDSDGSFFLYSKLTVDKSRWQQGNVFSCSVMHEALHNHYTQKSLSLSPG |
| | K (SEQ ID NO: 80) |
| Human IgG3 CH | ASTKGPSVFPLAPCSRSTSGGTAALGCLVKDYFPEPVTVSWNSGALTSGVHTFPA |
| | VLQSSGLYSLSSVVTVPSSSLGTQTYTCNVNHKPSNTKVDKRVELKTPLGDTTHT |
| | CPRCPEPKSCDTPPPCPRCPEPKSCDTPPPCPRCPEPKSCDTPPPCPRCPAPELL |
| | GGPSVFLFPPKPKDTLMISRTPEVTCVVVDVSHEDPEVQFKWYVDGVEVHNAKT |
| | KPREEQYNSTFRVVSVLTVLHQDWLNGKEYKCKVSNKALPAPIEKTISKTKGQP |
| | REPQVYTLPPSREEMTKNQVSLTCLVKGFYPSDIAVEWESSGQPENNYNTTPPM |
| | LDSDGSFFLYSKLTVDKSRWQQGNIFSCSVMHEALHNRFTQKSLSLSPGK (SEQ |
| | ID NO: 81) |
| Human IgG4 CH | ASTKGPSVFPLAPCSRSTSESTAALGCLVKDYFPEPVTVSWNSGALTSGVHTFPA |
| | VLQSSGLYSLSSVVTVPSSSLGTKTYTCNVDHKPSNTKVDKRVESKYGPPCPSCP |
| | APEFLGGPSVFLFPPKPKDTLMISRTPEVTCVVVDVSQEDPEVQFNWYVDGVEV |
| | HNAKTKPREEQFNSTYRVVSVLTVLHQDWLNGKEYKCKVSNKGLPSSIEKTISK |
| | AKGQPREPQVYTLPPSQEEMTKNQVSLTCLVKGFYPSDIAVEWESNGQPENNYK |
| | TTPPVLDSDGSFFLYSRLTVDKSRWQEGNVFSCSVMHEALHNHYTQKSLSLSLG |
| | K (SEQ ID NO: 82) |
| Human IgG1 CH1 | ASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKV (SEQ ID NO: 61) |
| Human IgG2 CH1 | ASTKGPSVFPLAPCSRSTSESTAALGCLVKDYFPEPVTVSWNSGALTSGVHTFPA |
| | VLQSSGLYSLSSVVTVPSSNFGTQTYTCNVDHKPSNTKVDKTV (SEQ ID NO: 83) |
| Human IgG3 CH1 | ASTKGPSVFPLAPCSRSTSGGTAALGCLVKDYFPEPVTVSWNSGALTSGVHTFPA |
| | VLQSSGLYSLSSVVTVPSSSLGTQTYTCNVNHKPSNTKVDKRV (SEQ ID NO: 84) |
| Human IgG4 CH1 | ASTKGPSVFPLAPCSRSTSESTAALGCLVKDYFPEPVTVSWNSGALTSGVHTFPA |
| | VLQSSGLYSLSSVVTVPSSSLGTKTYTCNVDHKPSNTKVDKRV (SEQ ID NO: 85) |
| Human IgG1 Fc | EPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEA |
| | LHNHYTQKSLSLSPGK (SEQ ID NO: 65) |
| Human IgG2 Fc | ERKCCVECPPCPAPPVAGPSVFLFPPKPKDTLMISRTPEVTCVVVDVSHEDPEVQ |
| | FNWYVDGVEVHNAKTKPREEQFNSTFRVVSVLTVVHQDWLNGKEYKCKVSNK |
| | GLPAPIEKTISKTKGQPREPQVYTLPPSREEMTKNQVSLTCLVKGFYPSDISVEWE |
| | SNGQPENNYKTTPPMLDSDGSFFLYSKLTVDKSRWQQGNVFSCSVMHEALHNH |
| | YTQKSLSLSPGK (SEQ ID NO: 86) |
| Human IgG3 Fc | ELKTPLGDTTHTCPRCPEPKSCDTPPPCPRCPEPKSCDTPPPCPRCPEPKSCDTPPP |
| | CPRCPAPELLGGPSVFLFPPKPKDTLMISRTPEVTCVVVDVSHEDPEVQFKWYVD |
| | GVEVHNAKTKPREEQYNSTFRVVSVLTVLHQDWLNGKEYKCKVSNKALPAPIE |
| | KTISKTKGQPREPQVYTLPPSREEMTKNQVSLTCLVKGFYPSDIAVEWESSGQPE |
| | NNYNTTPPMLDSDGSFFLYSKLTVDKSRWQQGNIFSCSVMHEALHNRFTQKSLS |
| | LSPGK (SEQ ID NO: 87) |
| Human IgG4 Fc | ESKYGPPCPSCPAPEFLGGPSVFLFPPKPKDTLMISRTPEVTCVVVDVSQEDPEVQ |
| | FNWYVDGVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFYPSDIAVEW |
| | ESNGQPENNYKTTPPVLDSDGSFFLYSRLTVDKSRWQEGNVFSCSVMHEALHN |
| | HYTQKSLSLSLGK (SEQ ID NO: 88) |
| Human IgG1 | EPKSCDKTHTCPPCP (SEQ ID NO: 70) |
| Hinge | |
| Human IgG2 | ERKCCVECPPCP (SEQ ID NO: 89) |
| Hinge | |
| Human IgG3 | ELKTPLGDTTHTCPRCPEPKSCDTPPPCPRCPEPKSCDTPPPCPRCPEPKSCDTPPP |
| Hinge | CPRCP (SEQ ID NO: 90) |
| Human IgG4 | ESKYGPPCPSCP (SEQ ID NO: 91) |
| Hinge | |

The term “variable region” refers to a portion of the light or heavy chains of an immunoglobulin that is generally located at the amino-terminal of the light or heavy chain and used in the binding and specificity of each particular antibody for its particular antigen. The variable region of a light chain is referred to as a “light chain variable region” or “VL region,” which includes at least one, typically one, “light chain variable domain” or “VL.” The variable region of a heavy chain is referred to as a “heavy chain variable region” or “VH region,” which includes at least one, typically one, “heavy chain variable domain” or “VH.” The variable domains differ extensively in sequence between different antibodies. A pair of VL and VH can associate and form a binding site that specifically binds the target antigen or epitope.

The VH and VL regions can be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). The variability in sequence is concentrated in the CDRs while the less variable portions in the variable domain are referred to as framework regions (FR). The CDRs of the light and heavy chains are primarily responsible for the interaction of the antibody with antigen. Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk, J Mol Biol 1987; 196:901-17).

A CDR refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains (Kabat et al, J. Biol. Chem. 252:6609-6616 (1977); Kabat, Adv. Prot. Chem. 32: 1-75 (1978)). Software programs (e.g., abYsis) are available and known to those of skill in the art for analysis of antibody sequence and determination of CDRs.

Bispecific antibodies of different structures are disclosed herein, which include VHs and VLs that specifically bind to human TNFα or human IL23p19. While specific anti-human TNFα and anti-human IL23p19 VH/VLs are exemplified herein, a person of ordinary in the art would understand that bispecific antibodies disclosed herein are not limited to the exemplified VH/VLs. Expressly contemplated herein also include variants of the bispecific antibodies disclosed herein in which the exemplified anti-human TNFα and anti-human IL23p19 VH/VLs are replaced with other anti-human TNFα and anti-human IL23p19 VH/VLs. A list of exemplary anti-human TNFα or anti-human IL23p19 VH/VLs is provided below as Table 2A or Table 2B, respectively.

TABLE 2A
Amino acid sequences of VH/VLs of exemplified anti-human TNFα antibodies

| | VL | VH |
|---|---|---|
| Adalimumab | DIQMTQSPSSLSASVGDRVTITCRASQG | EVQLVESGGGLVQPGRSLRLSCAASGFT |
| | IRNYLAWYQQKPGKAPKLLIYAASTLQS | FDDYAMHWVRQAPGKGLEWVSAITWNS |
| | GVPSRFSGSGSGTDFTLTISSLQPEDVA | GHIDYADSVEGRFTISRDNAKNSLYLQ |
| | TYYCQRYNRAPYTFGQGTKVEIK | MNSLRAEDTAVYYCAKVSYLSTASSLD |
| | (SEQ ID NO: 1) | YWGQGTLVTVSS |
| | | (SEQ ID NO: 2) |
| Golimumab | EIVLTQSPATLSLSPGERATLSCRASQS | QVQLVESGGGVVQPGRSLRLSCAASGF |
| | VYSYLAWYQQKPGQAPRLLIYDASNRAT | IFSSYAMHWVRQAPGNGLEWVAFMSYD |
| | GIPARFSGSGSGTDFTLTISSLEPEDFA | GSNKKYADSVKGRFTISRDNSKNTLYL |
| | VYYCQQRSNWPPFTFGPGTKVDIK | QMNSLRAEDTAVYYCARDRGIAAGGN |
| | (SEQ ID NO: 5) | YYYYGMDVWGQGTTVTVSS |
| | | (SEQ ID NO: 6) |
| Ozoralizumab | N/A | EVQLVESGGGLVQPGGSLRLSCAASGF |
| (VHH) | | TFSDYWMYWVRQAPGKGLEWVSEINTN |
| | | GLITKYPDSVKGRFTISRDNAKNTLYL |
| | | QMNSLRPEDTAVYYCARSPSGFNRGQG |
| | | TLVTVSS |
| | | (SEQ ID NO: 9) |

TABLE 2B
Amino acid sequences of VH/VLs of exemplified
anti-human IL23p19 antibodies

| | VL | VH |
|---|---|---|
| Guselkumab | QSVLTQPPSVSGAPG | EVQLVQSGAEVKKPG |
| | QRVTISCTGSSSNIG | ESLKISCKGSGYSFS |
| | SGYDVHWYQQLPGTA | NYWIGWVRQMPGKGL |
| | PKLLIYGNSKRPSGV | EWMGIIDPSNSYTRY |
| | PDRFSGSKSGTSASL | SPSFQGQVTISADKS |
| | AITGLQSEDEADYYC | ISTAYLQWSSLKASD |
| | ASWTDGLSLVVFGGG | TAMYYCARWYYKPFD |
| | TKLTVL | VWGQGTLVTVSS |
| | (SEQ ID NO: 10) | (SEQ ID NO: 11) |
| Guselkumab | QSVLTQPPSVSGAPG | EVQLVQSGAEVKKPG |
| variant | QRVTISCTGSSSNIG | ESLKISCKGSGYSFS |
| | SGYDVHWYQQLPGTA | NYWIGWVRQMPGKCL |
| | PKLLIYGNSKRPSGV | EWMGIIDPSNSYTRY |
| | PDRFSGSKSGTSASL | SPSFQGQVTISADKS |
| | AITGLQSEDEADYYC | ISTAYLQWSSLKASD |
| | ASWTDGLSLVVFGCG | TAMYYCARWYYKPFD |
| | TKLTVL | VWGQGTLVTVSS |
| | (SEQ ID NO: 93) | (SEQ ID NO: 94) |
| Tildrakizumab | DIQMTQSPSSLSASV | QVQLVQSGAEVKKPG |
| | GDRVTITCRTSENIY | ASVKVSCKASGYIFI |
| | SYLAWYQQKPGKAPK | TYWMTWVRQAPGQGL |
| | LLIYNAKTLAEGVPS | EWMGQIFPASGSADY |
| | RFSGSGSGTDFTLTI | NEKFEGRVTMTTDTS |
| | SSLQPEDFATYYCQH | TSTAYMELRSLRSDD |
| | HYGIPFTFGQGTKVE | TAVYYCARGGGGFAY |
| | IK | WGQGTLVTVSS |
| | (SEQ ID NO: 14) | (SEQ ID NO: 15) |

### 2.2 CrossMab-KIH

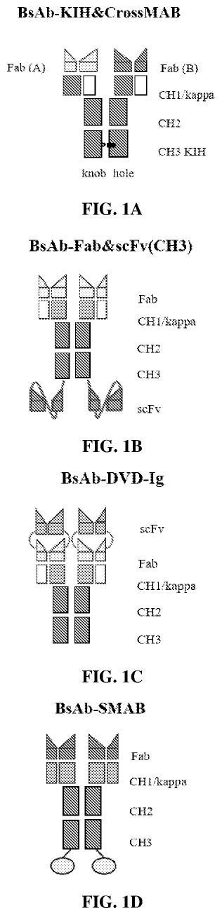

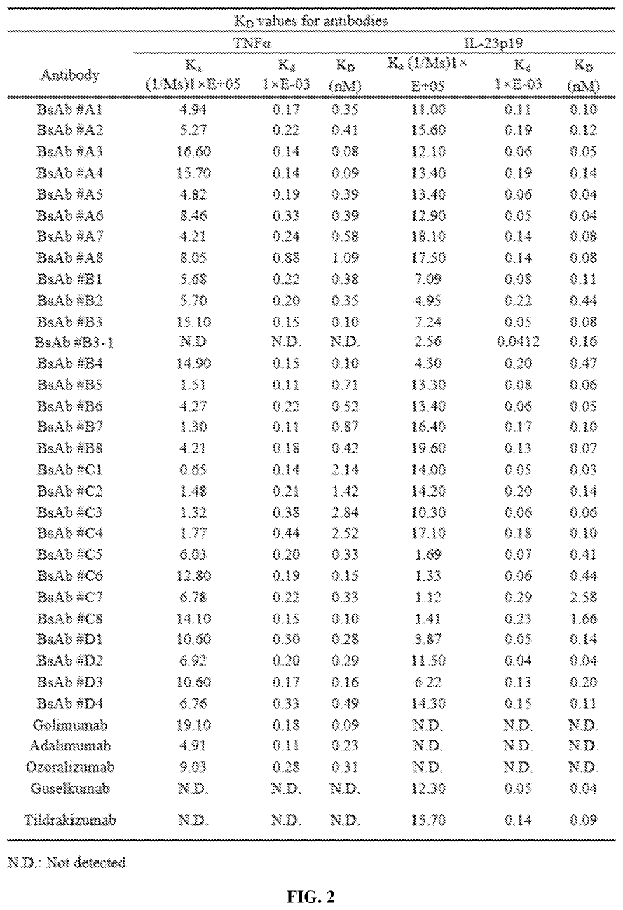

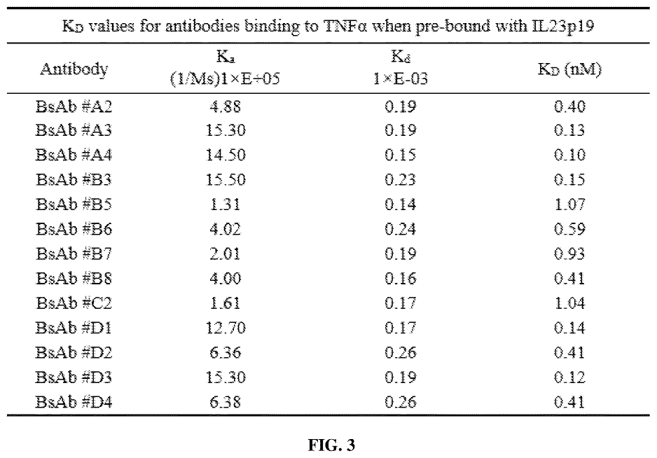

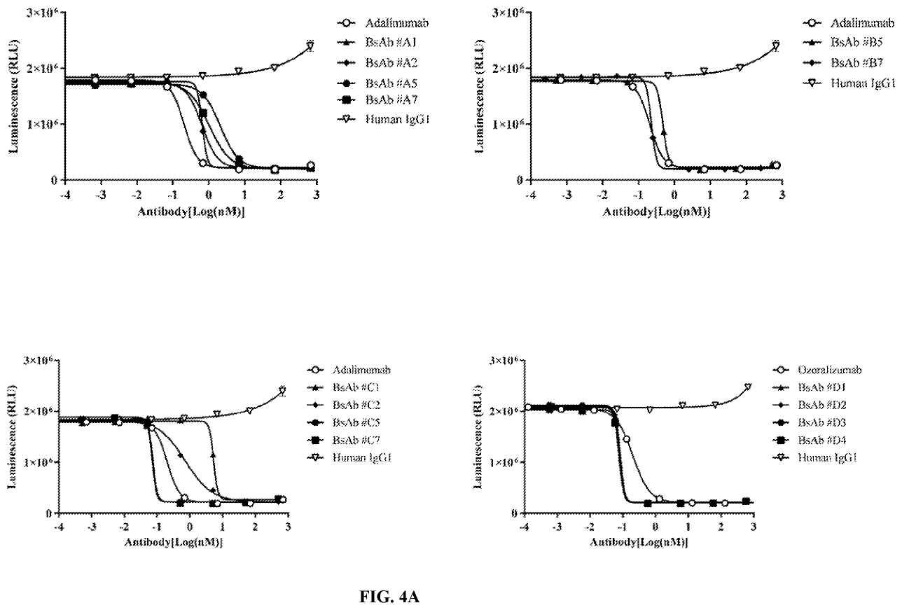

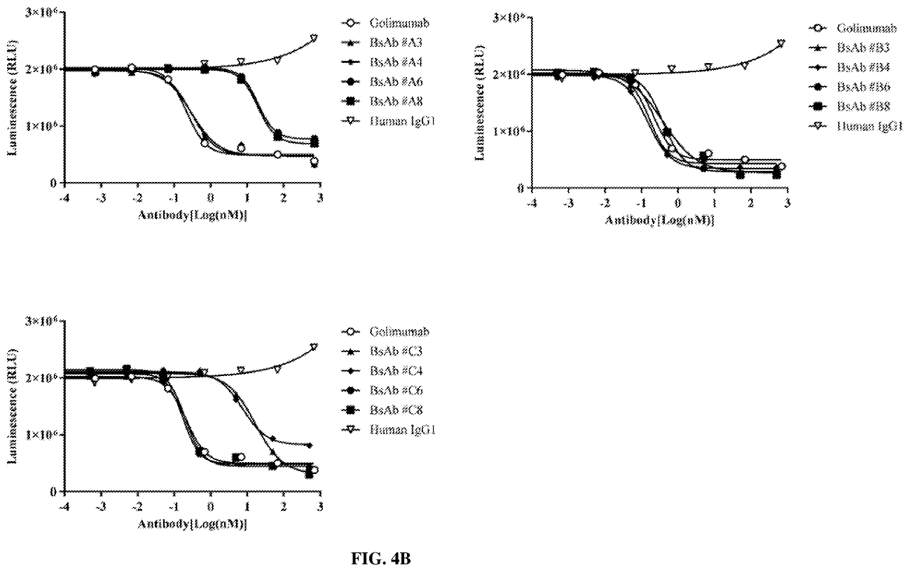

In some embodiments, the bispecific antibodies provided herein that specifically bind to human TNFα and to human IL23p19 have the “CrossMab-KIH” structure depicted in FIG. 1A. The “knobs-into-holes” or “KIH” model promotes formation of heterodimers of the engineered bispecific antibody instead of heavy chain homodimers.

The modification promoting the association of a pair of Fc domains in a bispecific antibody includes the so-called “knob-into-hole” modification, comprising a “knob” modification in one Fc domain and a “hole” modification in the other one. The knob-into-hole technology is described e.g., in U.S. Pat. Nos. 5,731,168; 7,695,936; Ridgway et al., Prot. Eng. 9, 617-621 (1996) and Carter, J Immunol. Meth. 248, 7-15 (2001). Generally, the method involves introducing a protuberance (“knob”) at the interface of a first Fc (the “Knob-Fc”) and a corresponding cavity (“hole”) in the interface of a second Fc (the “Hole-Fc”), such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, a “Knob-Fc region” and a “Hole-Fc region” are designed to form heterodimer pair. The Knob-Fc region refers to the Fc region in which an amino acid of the CH3 domain is replaced with an amino acid residue having a larger side chain volume, generating a protuberance within the CH3 domain positionable in a cavity within the CH3 domain of the Hole-Fc region, in which an amino acid residue of the CH3 domain is replaced with an amino acid residue having a smaller side chain volume, thereby generating a cavity within the CH3 domain within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis.

In some embodiments, the threonine residue at position 366 of the Knob-Fc region is replaced with a tryptophan residue (T366W), and the tyrosine residue at position 407 of the Hole-Fc region is replaced with a valine residue (Y407V), and optionally the threonine residue at position 366 is replaced with a serine residue (T366S) and the leucine residue at position 368 is replaced with an alanine residue (L368A). In some embodiments, the Knob-Fc region additionally has the serine residue at position 354 replaced with a cysteine residue (S354C), or the glutamic acid residue at position 356 replaced with a cysteine residue (E356C), and the Hole-Fc region additionally has the tyrosine residue at position 349 is replaced by a cysteine residue (Y349C). In some embodiments, the Knob-Fc region contains the amino acid substitutions S354C and T366W, and the Hole-Fc region contains the amino acid substitutions Y349C, T366S, L368A and Y407V. All amino acid residues are numbered according to the EU index.

The CrossMab design is used for resolving the BsAb light chain mismatch by exchanging one side of CL and CH1. By swapping the domains of one side heavy chain and light chain, the BsAb light chain can be assembled correctly.

Accordingly, in some embodiments, the bispecific antibodies provided herein can have two pairs of light chains and heavy chains. In some embodiments, the first pair of light chain and heavy chain (LC1 and HC1) specifically binds to human TNFα and the second pair of light chain and heavy chain (LC2 and HC2) specifically binds to human IL23p19. In some embodiments, the LC1 and HC1 pair specifically binds to human IL23p19 and the LC2 and HC2 pair specifically binds to human TNFα. The HC1 and HC2 pair of the bispecific antibodies can adopts a KIH design, wherein HC1 includes a Knob-Fc region and the HC2 includes a Hole-Fc region. Alternatively, in some embodiments, the HC2 can include a Knob-Fc region and the HC1 can include a Hole-Fc region. Additionally, the light chain constant region (CL region) of LC1 and the heavy chain constant domain 1 (CH1) of HC1 are swapped to avoid light chain mismatch. As such, the bispecific antibodies provided herein can have LC1, HC1, LC2, and HC2, wherein (1) the LC1/HC1 pair have swapped constant domains, (2) the LC2/HC2 pair have normal constant domains; and (3) the HC1/HC2 can have the KIH structure. In some embodiments, HC1 has a Knob-Fc region and HC2 has a Hole-Fc region. In some embodiments, HC1 has a Hole-Fc region and HC2 has a Knob-Fc region.

In some embodiments, the bispecific antibodies that specifically bind to human TNFα and to human IL23p19 provided herein can have four polypeptides, including: (1) a first light chain (LC1) comprising a first light chain variable domain (VL1) and a first heavy chain constant domain 1 (CH1); (2) a first heavy chain (HC1) comprising a first heavy chain variable domain (VH1), a first light chain constant region (CL), and a Knob-Fc region; (3) a second light chain (LC2) comprising a second light chain variable domain (VL2) and a second CL region; and (4) a second heavy chain (HC2) comprising a second heavy chain variable domain (VH2), a second CH1 domain, and a Hole-Fc region; wherein (i) the VL1/VH1 pair and the VL2/VH2 pair specifically bind to human TNFα and human IL23p19, respectively, or to human IL23p19 and human TNFα, respectively; and (ii) the Knob-Fc region is a human Fc region variant having a T366W substitution (numbered according to the EU Index); and the Hole-Fc region is a human Fc region variant having a Y407V substitution (numbered according to the EU Index).

The amino acid sequences of the CH1, the CL region, and the Fc region (CH2 and CH3) of the bispecific antibodies disclosed herein can be derived from any appropriate source, e.g., a constant region of an antibody such as an IgG1, IgG2, IgG3, or IgG4. Antibody heavy and light chain constant regions amino acid sequences are well known in the art, e.g., those provided in the IMGT database (www.imgt.org) or at www.vbase2.org/vbstat.php., both of which are incorporated by reference herein.

In some embodiments, the constant domains and constant regions of the bispecific antibodies provided herein are derived from human IgG. In some embodiments, the constant domains and constant regions of the bispecific antibodies provided herein are derived from human IgG1. In some embodiments, the constant domains and constant regions of the bispecific antibodies provided herein are derived from human IgG2. In some embodiments, the constant domains and constant regions of the bispecific antibodies provided herein are derived from human IgG3. In some embodiments, the constant domains and constant regions of the bispecific antibodies provided herein are derived from human IgG4. In some embodiments, the amino acid sequences of the CH1, the CL region, and the Fc region (hinge, CH2 and CH3) of the bispecific antibodies disclosed herein can comprise one or more amino acid substitutions that differ from the wild type immunoglobulin, e.g., one or more amino acid substitutions in a wild type IgG1 or IgG4. Such substitutions are known in the art (see, e.g., U.S. Pat. Nos. 7,704,497, 7,083,784, 6,821,505, 8,323,962, 6,737,056, and 7,416,727).

In some embodiments, the first and second CH1 domains can be independently selected from the group consisting of a human IgG1 CH1 domain (SEQ ID NO: 61), a human IgG2 CH1 domain (SEQ ID NO: 83), a human IgG3 CH1 domain (SEQ ID NO: 84), and a human IgG4 CH1 domain (SEQ ID NO: 85). In some embodiments, the first and second CH1 domains are both human IgG1 CH1 domain (SEQ ID NO: 61).

In some embodiments, the CL region can be kappa CL (Cκ; SEQ ID NO: 68). In some embodiments, the CL region can be lambda CL (Ck, SEQ ID NO: 69). In some embodiments, the first CL region is Cκ (SEQ ID NO: 68). In some embodiments, the first CL region is Cλ (SEQ ID NO: 69). In some embodiments, the second CL region is Cκ (SEQ ID NO: 68). In some embodiments, the second CL is Cλ (SEQ ID NO: 69). In some embodiments, the first CL region is Cλ (SEQ ID NO: 69) and the second CL region is Cκ (SEQ ID NO: 68). In some embodiments, the first CL region is Ck (SEQ ID NO: 69) and the second CL region is Cλ (SEQ ID NO: 69). In some embodiments, the first CL region is Cκ (SEQ ID NO: 68) and the second CL region is Cκ (SEQ ID NO: 68). In some embodiments, the first CL region is Cκ (SEQ ID NO: 68) and the second CL region is Cλ (SEQ ID NO: 69).

In some embodiments, the hinge of the Fc region can be independently selected from a group consisting of a human IgG1 hinge region (SEQ ID NO: 70), a human IgG2 hinge region (SEQ ID NO: 89), a human IgG3 hinge region (SEQ ID NO: 90), and a human IgG4 hinge region (SEQ ID NO: 91).

In some embodiments, the Fc regions of the bispecific antibodies provided herein can be variants of the Fc region of human IgG1. In some embodiments, the Knob-Fc region is human IgG1 Fc having a T366W substitution. In some embodiments, the Hole-Fc region is human IgG1 Fc having a Y407T substitution. In some embodiments, the Knob-Fc and Hole-Fc regions can further include S354C and Y349C substitutions, respectively. In some embodiments, the Knob-Fc and Hole-Fc regions can further include E356C and Y349C substitutions, respectively. In some embodiments, the Hole-Fc region can further include T366S and L368A substitutions. All amino acid residues are numbered according to the EU Index. In some embodiments, the Knob-Fc region can have the amino acid sequence of SEQ ID NO: 66. In some embodiments, the Hole-Fc region can have the amino acid sequence of SEQ ID NO: 67. A list of exemplary Fc sequences in KIH models is provided below as Table 3.

TABLE 3
Exemplified Fc sequences in KIH models.

| Exemplary Fc in | |
|---|---|
| KIH model | Sequences |
| Fc chain 1 | EPKSCDKTHTCPPCPAPELLGGPSVFLFP |
| (knob): S354C | PKPKDTLMISRTPEVTCVVVDVSHEDPEV |
| and T366W | KFNWYVDGVEVHNAKTKPREEQYNSTYRV |
| | VSVLTVLHQDWLNGKEYKCKVSNKALPAP |
| | IEKTISKAKGQPREPQVYTLPPCRDELTK |
| | NQVSLWCLVKGFYPSDIAVEWESNGQPEN |
| | NYKTTPPVLDSDGSFFLYSKLTVDKSRWQ |
| | QGNVFSCSVMHEALHNHYTQKSLSLSPGK |
| | (SEQ ID NO: 66) |
| Fc chain 2 | EPKSCDKTHTCPPCPAPELLGGPSVFLFP |
| (hole): Y349C, | PKPKDTLMISRTPEVTCVVVDVSHEDPEV |
| T366S, L368A, | KFNWYVDGVEVHNAKTKPREEQYNSTYRV |
| Y407V | VSVLTVLHQDWLNGKEYKCKVSNKALPAP |
| | IEKTISKAKGQPREPQVCTLPPSRDELTK |
| | NQVSLSCAVKGFYPSDIAVEWESNGQPEN |
| | NYKTTPPVLDSDGSFFLVSKLTVDKSRWQ |
| | QGNVFSCSVMHEALHNHYTQKSLSLSPGK |
| | (SEQ ID NO: 67) |

In some embodiments of the bispecific antibodies provided herein, (1) the first CL region is kappa CL (Cκ; SEQ ID NO: 68) or lambda CL (Cλ, SEQ ID NO: 69); and the second CL region is Cκ (SEQ ID NO: 68) or Cλ (SEQ ID NO: 69); (2) the first CH1 domain and the second CH1 domain are both human IgG1 CH1 domain (SEQ ID NO: 61); or (3) the Knob-Fc region has the amino acid sequence of SEQ ID NO: 66; and the Hole-Fc region has the amino acid sequence of SEQ ID NO: 67; or any combination of (1)-(3).

In some embodiments of the bispecific antibodies provided herein, the VL1/VH1 pair specifically binds to human TNFα and the VL2/VH2 pair specifically binds to human IL23p19. The VL1/VH1 pair can be any VL/VH pair that specifically binds to human TNFα. In some embodiments, the VL1 and VH1 are the VL and VH of adalimumab and have the amino acid sequences of SEQ ID NOs: 1 and 2, respectively. In some embodiments, the VL1 and VH1 are the VL and VH of golimumab and have the amino acid sequences of SEQ ID NOs: 5 and 6, respectively. The VL2/VH2 pair can be any VL/VH pair that specifically binds to human IL23p19. In some embodiments, the VL2 and VH2 are the VL and VH of guselkumab and have the amino acid sequences of SEQ ID NOs: 10 and 11, respectively. In some embodiments, the VL2 and VH2 are the VL and VH of tildrakizumab and have the amino acid sequences of SEQ ID NOs: 14 and 15, respectively.

In some embodiments of the bispecific antibodies provided herein, the VL1 and VH1 have the amino acid sequences of (1) SEQ ID NOs: 1 and 2, respectively; or (2) SEQ ID NOs: 5 and 6, respectively. In some embodiments, the VL1 and VH1 have the amino acid sequences of SEQ ID NOs: 1 and 2, respectively. In some embodiments, the VL1 and VH1 have the amino acid sequences of SEQ ID NOs: 5 and 6, respectively. In some embodiments of the bispecific antibodies provided herein, the VL2 and VH2 have the amino acid sequences of (1) SEQ ID NOs: 10 and 11, respectively; or (2) SEQ ID NOs: 14 and 15, respectively. In some embodiments, the VL2 and VH2 have the amino acid sequences of SEQ ID NOs: 10 and 11, respectively. In some embodiments, the VL2 and VH2 have the amino acid sequences of SEQ ID NOs: 14 and 15, respectively.

In some embodiments of the bispecific antibodies provided herein, the VL1, VH1, VL2 and VH2 have the amino acid sequences of (1) SEQ ID NOs: 1, 2, 10, and 11, respectively; (2) SEQ ID NOs: 1, 2, 14, and 15, respectively; (3) SEQ ID NOs: 5, 6, 10, and 11, respectively; or (4) SEQ ID NOs: 5, 6, 14, and 15, respectively. In some embodiments, the VL1, VH1, VL2 and VH2 have the amino acid sequences of SEQ ID NOs: 1, 2, 10, and 11, respectively. In some embodiments, the VL1, VH1, VL2 and VH2 have the amino acid sequences of SEQ ID NOs: 1, 2, 14, and 15, respectively.

In some embodiments, the VL1, VHI, VL2 and VH2 have the amino acid sequences of SEQ ID NOs: 5, 6, 10, and 11, respectively. In some embodiments, the VL1, VH1, VL2 and VH2 have the amino acid sequences of SEQ ID NOs: 5, 6, 14, and 15, respectively.

In some embodiments of the bispecific antibodies provided herein, the VL1/VHI pair specifically binds to human IL23p19 and the VL2/VH2 pair specifically binds to human TNFα. The VL1/VH1 pair can be any VL/VH pair that specifically binds to human IL23p19. In some embodiments, the VL1 and VH1 are the VL and VH of guselkumab and have the amino acid sequences of SEQ ID NOs: 10 and 11, respectively. In some embodiments, the VL1 and VHI are the VL and VH of tildrakizumab and have the amino acid sequences of SEQ ID NOs: 14 and 15, respectively. The VL2/VH2 pair can be any VL/VH pair that specifically binds to human TNFα. In some embodiments, the VL2 and VH2 are the VL and VH of adalimumab and have the amino acid sequences of SEQ ID NOs: 1 and 2, respectively. In some embodiments, the VL2 and VH2 are the VL and VH of golimumab and have the amino acid sequences of SEQ ID NOs: 5 and 6, respectively.

In some embodiments of the bispecific antibodies provided herein, the VL1 and VH1 have the amino acid sequences of (1) SEQ ID NOs: 10 and 11, respectively; or (2) SEQ ID NOs: 14 and 15, respectively. In some embodiments, the VL1 and VHI have the amino acid sequences of SEQ ID NOs: 10 and 11, respectively. In some embodiments, the VL1 and VH1 have the amino acid sequences of SEQ ID NOs: 14 and 15, respectively. In some embodiments of the bispecific antibodies provided herein, the VL2 and VH2 have the amino acid sequences of (1) SEQ ID NOs: 1 and 2, respectively; or (2) SEQ ID NOs: 5 and 6, respectively. In some embodiments, the VL2 and VH2 have the amino acid sequences of SEQ ID NOs: 1 and 2, respectively. In some embodiments, the VL2 and VH2 have the amino acid sequences of SEQ ID NOs: 5 and 6, respectively.

In some embodiments of the bispecific antibodies provided herein, the VL1, VH1, VL2 and VH-2 have the amino acid sequences of (1) SEQ ID NOs: 10, 11, 1, and 2, respectively; (2) SEQ ID NOs: 10, 11, 5, and 6, respectively; (3) SEQ ID NOs: 14, 15, 1, and 2, respectively, or (4) SEQ ID NOs: 14, 15, 5, and 6, respectively. In some embodiments, the VL1, VH1, VL2 and VH2 have the amino acid sequences of SEQ ID NOs: 10, 11, 1, and 2, respectively. In some embodiments, the VL1, VH1, VL2 and VH2 have the amino acid sequences of SEQ ID NOs: 10, 11, 5, and 6, respectively.

In some embodiments, the VL1, VH1, VL2 and VH-2 have the amino acid sequences of SEQ ID NOs: 14, 15, 1, and 2, respectively. In some embodiments, the VL1, VH1, VL2 and VH2 have the amino acid sequences of SEQ ID NOs: 14, 15, 5, and 6, respectively. A list of exemplary VL1, VH1, VL2 and VH-2 of bispecific antibodies is provided below as Table 4A.

TABLE 4A
Exemplified Bispecific Antibodies to human TNFα and human IL23p19

| | | |
|---|---|---|
| A1 | LC1 | LC2 |
| | QSVLTQPPSVSGAPGQRVTISCTGSSSNIGS | DIQMTQSPSSLSASVGDRVTITCRASQGIR |
| | GYDVHWYQQLPGTAPKLLIYGNSKRPSGV | NYLAWYQQKPGKAPKLLIYAASTLQSGVP |
| | PDRFSGSKSGTSASLAITGLQSEDEADYYC | SRFSGSGSGTDFTLTISSLQPEDVATYYCQ |
| | ASWTDGLSLVVFGGGTKLTVLASTKGPSV | RYNRAPYTFGQGTKVEIKRTVAAPSVFIFP |
| | FPLAPSSKSTSGGTAALGCLVKDYFPEPVT | PSDEQLKSGTASVVCLLNNFYPREAKVQW |
| | VSWNSGALTSGVHTFPAVLQSSGLYSLSS | KVDNALQSGNSQESVTEQDSKDSTYSLSS |
| | VVTVPSSSLGTQTYICNVNHKPSNTKVDK | TLTLSKADYEKHKVYACEVTHQGLSSPVT |
| | KV | KSFNRGEC |
| | (SEQ ID NO: 18) | (SEQ ID NO: 3) |
| | HC1 | HC2 |
| | EVQLVQSGAEVKKPGESLKISCKGSGYSFS | EVQLVESGGGLVQPGRSLRLSCAASGFTF |
| | NYWIGWVRQMPGKGLEWMGIIDPSNSYT | DDYAMHWVRQAPGKGLEWVSAITWNSG |
| | RYSPSFQGQVTISADKSISTAYLQWSSLKA | HIDYADSVEGRFTISRDNAKNSLYLQMNS |
| | SDTAMYYCARWYYKPFDVWGQGTLVTV | LRAEDTAVYYCAKVSYLSTASSLDYWGQ |
| | SSGQPKAAPSVTLFPPSSEELQANKATLVC | GTLVTVSSASTKGPSVFPLAPSSKSTSGGT |
| | LISDFYPGAVTVAWKADSSPVKAGVETTT | AALGCLVKDYFPEPVTVSWNSGALTSGVH |
| | PSKQSNNKYAASSYLSLTPEQWKSHRSYS | TFPAVLQSSGLYSLSSVVTVPSSSLGTQTYI |
| | CQVTHEGSTVEKTVAPTECSEPKSCDKTH | CNVNHKPSNTKVDKKVEPKSCDKTHTCPP |
| | TCPPCPAPELLGGPSVFLFPPKPKDTLMISR | CPAPELLGGPSVFLFPPKPKDTLMISRTPEV |
| | TPEVTCVVVDVSHEDPEVKFNWYVDGVE | TCVVVDVSHEDPEVKFNWYVDGVEVHNA |
| | VHNAKTKPREEQYNSTYRVVSVLTVLHQ | KTKPREEQYNSTYRVVSVLTVLHQDWLN |
| | DWLNGKEYKCKVSNKALPAPIEKTISKAK | GKEYKCKVSNKALPAPIEKTISKAKGQPRE |
| | GQPREPQVYTLPPCRDELTKNQVSLWCLV | PQVCTLPPSRDELTKNQVSLSCAVKGFYPS |
| | KGFYPSDIAVEWESNGQPENNYKTTPPVL | DIAVEWESNGQPENNYKTTPPVLDSDGSF |
| | DSDGSFFLYSKLTVDKSRWQQGNVFSCSV | FLVSKLTVDKSRWQQGNVFSCSVMHEAL |
| | MHEALHNHYTQKSLSLSPGK | HNHYTQKSLSLSPGK |
| | (SEQ ID NO: 19) | (SEQ ID NO: 20) |
| A2 | LC1 | LC2 |
| | DIQMTQSPSSLSASVGDRVTITCRTSENIYS | DIQMTQSPSSLSASVGDRVTITCRASQGIR |
| | YLAWYQQKPGKAPKLLIYNAKTLAEGVPS | NYLAWYQQKPGKAPKLLIYAASTLQSGVP |
| | RFSGSGSGTDFTLTISSLQPEDFATYYCQH | SRFSGSGSGTDFTLTISSLQPEDVATYYCQ |
| | HYGIPFTFGQGTKVEIKASTKGPSVFPLAPS | RYNRAPYTFGQGTKVEIKRTVAAPSVFIFP |
| | SKSTSGGTAALGCLVKDYFPEPVTVSWNS | PSDEQLKSGTASVVCLLNNFYPREAKVQW |
| | GALTSGVHTFPAVLQSSGLYSLSSVVTVPS | KVDNALQSGNSQESVTEQDSKDSTYSLSS |
| | SSLGTQTYICNVNHKPSNTKVDKKV | TLTLSKADYEKHKVYACEVTHQGLSSPVT |
| | (SEQ ID NO: 21) | KSFNRGEC |
| | | (SEQ ID NO: 3) |
| | HC1 | HC2 |
| | QVQLVQSGAEVKKPGASVKVSCKASGYIF | EVQLVESGGGLVQPGRSLRLSCAASGFTF |
| | ITYWMTWVRQAPGQGLEWMGQIFPASGS | DDYAMHWVRQAPGKGLEWVSAITWNSG |
| | ADYNEKFEGRVTMTTDTSTSTAYMELRSL | HIDYADSVEGRFTISRDNAKNSLYLQMNS |
| | RSDDTAVYYCARGGGGFAYWGQGTLVT | LRAEDTAVYYCAKVSYLSTASSLDYWGQ |
| | VSSRTVAAPSVFIFPPSDEQLKSGTASVVC | GTLVTVSSASTKGPSVFPLAPSSKSTSGGT |
| | LLNNFYPREAKVQWKVDNALQSGNSQES | AALGCLVKDYFPEPVTVSWNSGALTSGVH |
| | VTEQDSKDSTYSLSSTLTLSKADYEKHKV | TFPAVLQSSGLYSLSSVVTVPSSSLGTQTYI |
| | YACEVTHQGLSSPVTKSFNRGECEPKSCD | CNVNHKPSNTKVDKKVEPKSCDKTHTCPP |
| | KTHTCPPCPAPELLGGPSVFLFPPKPKDTL | CPAPELLGGPSVFLFPPKPKDTLMISRTPEV |
| | MISRTPEVTCVVVDVSHEDPEVKFNWYVD | TCVVVDVSHEDPEVKFNWYVDGVEVHNA |
| | GVEVHNAKTKPREEQYNSTYRVVSVLTVL | KTKPREEQYNSTYRVVSVLTVLHQDWLN |
| | HQDWLNGKEYKCKVSNKALPAPIEKTISK | GKEYKCKVSNKALPAPIEKTISKAKGQPRE |
| | AKGQPREPQVYTLPPCRDELTKNQVSLWC | PQVCTLPPSRDELTKNQVSLSCAVKGFYPS |
| | LVKGFYPSDIAVEWESNGQPENNYKTTPP | DIAVEWESNGQPENNYKTTPPVLDSDGSF |
| | VLDSDGSFFLYSKLTVDKSRWQQGNVFSC | FLVSKLTVDKSRWQQGNVFSCSVMHEAL |
| | SVMHEALHNHYTQKSLSLSPGK | HNHYTQKSLSLSPGK |
| | (SEQ ID NO: 22) | (SEQ ID NO: 20) |
| A3 | LC1 | LC2 |
| | QSVLTQPPSVSGAPGQRVTISCTGSSSNIGS | EIVLTQSPATLSLSPGERATLSCRASQSVYS |
| | GYDVHWYQQLPGTAPKLLIYGNSKRPSGV | YLAWYQQKPGQAPRLLIYDASNRATGIPA |
| | PDRFSGSKSGTSASLAITGLQSEDEADYYC | RFSGSGSGTDFTLTISSLEPEDFAVYYCQQ |
| | ASWTDGLSLVVFGGGTKLTVLASTKGPSV | RSNWPPFTFGPGTKVDIKRTVAAPSVFIFPP |
| | FPLAPSSKSTSGGTAALGCLVKDYFPEPVT | SDEQLKSGTASVVCLLNNFYPREAKVQW |
| | VSWNSGALTSGVHTFPAVLQSSGLYSLSS | KVDNALQSGNSQESVTEQDSKDSTYSLSS |
| | VVTVPSSSLGTQTYICNVNHKPSNTKVDK | TLTLSKADYEKHKVYACEVTHQGLSSPVT |
| | KV | KSFNRGEC |
| | (SEQ ID NO: 18) | (SEQ ID NO: 7) |
| | HC1 | HC2 |
| | EVQLVQSGAEVKKPGESLKISCKGSGYSFS | QVQLVESGGGVVQPGRSLRLSCAASGFIFS |
| | NYWIGWVRQMPGKGLEWMGIIDPSNSYT | SYAMHWVRQAPGNGLEWVAFMSYDGSN |
| | RYSPSFQGQVTISADKSISTAYLQWSSLKA | KKYADSVKGRFTISRDNSKNTLYLQMNSL |
| | SDTAMYYCARWYYKPFDVWGQGTLVTV | RAEDTAVYYCARDRGIAAGGNYYYYGM |
| | SSGQPKAAPSVTLFPPSSEELQANKATLVC | DVWGQGTTVTVSSASTKGPSVFPLAPSSK |
| | LISDFYPGAVTVAWKADSSPVKAGVETTT | STSGGTAALGCLVKDYFPEPVTVSWNSGA |
| | PSKQSNNKYAASSYLSLTPEQWKSHRSYS | LTSGVHTFPAVLQSSGLYSLSSVVTVPSSS |
| | CQVTHEGSTVEKTVAPTECSEPKSCDKTH | LGTQTYICNVNHKPSNTKVDKKVEPKSCD |
| | TCPPCPAPELLGGPSVFLFPPKPKDTLMISR | KTHTCPPCPAPELLGGPSVFLFPPKPKDTL |
| | TPEVTCVVVDVSHEDPEVKFNWYVDGVE | MISRTPEVTCVVVDVSHEDPEVKFNWYVD |
| | VHNAKTKPREEQYNSTYRVVSVLTVLHQ | GVEVHNAKTKPREEQYNSTYRVVSVLTVL |
| | DWLNGKEYKCKVSNKALPAPIEKTISKAK | HQDWLNGKEYKCKVSNKALPAPIEKTISK |
| | GQPREPQVYTLPPCRDELTKNQVSLWCLV | AKGQPREPQVCTLPPSRDELTKNQVSLSC |
| | KGFYPSDIAVEWESNGQPENNYKTTPPVL | AVKGFYPSDIAVEWESNGQPENNYKTTPP |
| | DSDGSFFLYSKLTVDKSRWQQGNVFSCSV | VLDSDGSFFLVSKLTVDKSRWQQGNVFSC |
| | MHEALHNHYTQKSLSLSPGK | SVMHEALHNHYTQKSLSLSPGK |
| | (SEQ ID NO: 19) | (SEQ ID NO: 23) |
| A4 | LC1 | LC2 |
| | DIQMTQSPSSLSASVGDRVTITCRTSENIYS | EIVLTQSPATLSLSPGERATLSCRASQSVYS |
| | YLAWYQQKPGKAPKLLIYNAKTLAEGVPS | YLAWYQQKPGQAPRLLIYDASNRATGIPA |
| | RFSGSGSGTDFTLTISSLQPEDFATYYCQH | RFSGSGSGTDFTLTISSLEPEDFAVYYCQQ |
| | HYGIPFTFGQGTKVEIKASTKGPSVFPLAPS | RSNWPPFTFGPGTKVDIKRTVAAPSVFIFPP |
| | SKSTSGGTAALGCLVKDYFPEPVTVSWNS | SDEQLKSGTASVVCLLNNFYPREAKVQW |
| | GALTSGVHTFPAVLQSSGLYSLSSVVTVPS | KVDNALQSGNSQESVTEQDSKDSTYSLSS |
| | SSLGTQTYICNVNHKPSNTKVDKKV | TLTLSKADYEKHKVYACEVTHQGLSSPVT |
| | (SEQ ID NO: 21) | KSFNRGEC |
| | | (SEQ ID NO: 7) |
| | HC1 | HC2 |
| | QVQLVQSGAEVKKPGASVKVSCKASGYIF | QVQLVESGGGVVQPGRSLRLSCAASGFIFS |
| | ITYWMTWVRQAPGQGLEWMGQIFPASGS | SYAMHWVRQAPGNGLEWVAFMSYDGSN |
| | ADYNEKFEGRVTMTTDTSTSTAYMELRSL | KKYADSVKGRFTISRDNSKNTLYLQMNSL |
| | RSDDTAVYYCARGGGGFAYWGQGTLVT | RAEDTAVYYCARDRGIAAGGNYYYYGM |
| | VSSRTVAAPSVFIFPPSDEQLKSGTASVVC | DVWGQGTTVTVSSASTKGPSVFPLAPSSK |
| | LLNNFYPREAKVQWKVDNALQSGNSQES | STSGGTAALGCLVKDYFPEPVTVSWNSGA |
| | VTEQDSKDSTYSLSSTLTLSKADYEKHKV | LTSGVHTFPAVLQSSGLYSLSSVVTVPSSS |
| | YACEVTHQGLSSPVTKSFNRGECEPKSCD | LGTQTYICNVNHKPSNTKVDKKVEPKSCD |
| | KTHTCPPCPAPELLGGPSVFLFPPKPKDTL | KTHTCPPCPAPELLGGPSVFLFPPKPKDTL |
| | MISRTPEVTCVVVDVSHEDPEVKFNWYVD | MISRTPEVTCVVVDVSHEDPEVKFNWYVD |
| | GVEVHNAKTKPREEQYNSTYRVVSVLTVL | GVEVHNAKTKPREEQYNSTYRVVSVLTVL |
| | HQDWLNGKEYKCKVSNKALPAPIEKTISK | HQDWLNGKEYKCKVSNKALPAPIEKTISK |
| | AKGQPREPQVYTLPPCRDELTKNQVSLWC | AKGQPREPQVCTLPPSRDELTKNQVSLSC |
| | LVKGFYPSDIAVEWESNGQPENNYKTTPP | AVKGFYPSDIAVEWESNGQPENNYKTTPP |
| | VLDSDGSFFLYSKLTVDKSRWQQGNVFSC | VLDSDGSFFLVSKLTVDKSRWQQGNVFSC |
| | SVMHEALHNHYTQKSLSLSPGK | SVMHEALHNHYTQKSLSLSPGK |
| | (SEQ ID NO: 22) | (SEQ ID NO: 23) |
| A5 | LC1 | LC2 |
| | DIQMTQSPSSLSASVGDRVTITCRASQGIR | QSVLTQPPSVSGAPGQRVTISCTGSSSNIGS |
| | NYLAWYQQKPGKAPKLLIYAASTLQSGVP | GYDVHWYQQLPGTAPKLLIYGNSKRPSGV |
| | SRFSGSGSGTDFTLTISSLQPEDVATYYCQ | PDRFSGSKSGTSASLAITGLQSEDEADYYC |
| | RYNRAPYTFGQGTKVEIKASTKGPSVFPLA | ASWTDGLSLVVFGGGTKLTVLGQPKAAPS |
| | PSSKSTSGGTAALGCLVKDYFPEPVTVSW | VTLFPPSSEELQANKATLVCLISDFYPGAV |
| | NSGALTSGVHTFPAVLQSSGLYSLSSVVTV | TVAWKADSSPVKAGVETTTPSKQSNNKY |
| | PSSSLGTQTYICNVNHKPSNTKVDKKV | AASSYLSLTPEQWKSHRSYSCQVTHEGST |
| | (SEQ ID NO: 24) | VEKTVAPTECS |
| | | (SEQ ID NO: 12) |
| | HC1 | HC2 |
| | EVQLVESGGGLVQPGRSLRLSCAASGFTF | EVQLVQSGAEVKKPGESLKISCKGSGYSFS |
| | DDYAMHWVRQAPGKGLEWVSAITWNSG | NYWIGWVRQMPGKGLEWMGIIDPSNSYT |
| | HIDYADSVEGRFTISRDNAKNSLYLQMNS | RYSPSFQGQVTISADKSISTAYLQWSSLKA |
| | LRAEDTAVYYCAKVSYLSTASSLDYWGQ | SDTAMYYCARWYYKPFDVWGQGTLVTV |
| | GTLVTVSSRTVAAPSVFIFPPSDEQLKSGT | SSASTKGPSVFPLAPSSKSTSGGTAALGCL |
| | ASVVCLLNNFYPREAKVQWKVDNALQSG | VKDYFPEPVTVSWNSGALTSGVHTFPAVL |
| | NSQESVTEQDSKDSTYSLSSTLTLSKADYE | QSSGLYSLSSVVTVPSSSLGTQTYICNVNH |
| | KHKVYACEVTHQGLSSPVTKSFNRGECEP | KPSNTKVDKKVEPKSCDKTHTCPPCPAPE |
| | KSCDKTHTCPPCPAPELLGGPSVFLFPPKP | LLGGPSVFLFPPKPKDTLMISRTPEVTCVV |
| | KDTLMISRTPEVTCVVVDVSHEDPEVKFN | VDVSHEDPEVKFNWYVDGVEVHNAKTKP |
| | WYVDGVEVHNAKTKPREEQYNSTYRVVS | REEQYNSTYRVVSVLTVLHQDWLNGKEY |
| | VLTVLHQDWLNGKEYKCKVSNKALPAPIE | KCKVSNKALPAPIEKTISKAKGQPREPQVC |
| | KTISKAKGQPREPQVYTLPPCRDELTKNQ | TLPPSRDELTKNQVSLSCAVKGFYPSDIAV |
| | VSLWCLVKGFYPSDIAVEWESNGQPENNY | EWESNGQPENNYKTTPPVLDSDGSFFLVS |
| | KTTPPVLDSDGSFFLYSKLTVDKSRWQQG | KLTVDKSRWQQGNVFSCSVMHEALHNHY |
| | NVFSCSVMHEALHNHYTQKSLSLSPGK | TQKSLSLSPGK |
| | (SEQ ID NO: 25) | (SEQ ID NO: 26) |
| A6 | LC1 | LC2 |
| | EIVLTQSPATLSLSPGERATLSCRASQSVYS | QSVLTQPPSVSGAPGQRVTISCTGSSSNIGS |
| | YLAWYQQKPGQAPRLLIYDASNRATGIPA | GYDVHWYQQLPGTAPKLLIYGNSKRPSGV |
| | RFSGSGSGTDFTLTISSLEPEDFAVYYCQQ | PDRFSGSKSGTSASLAITGLQSEDEADYYC |
| | RSNWPPFTFGPGTKVDIKASTKGPSVFPLA | ASWTDGLSLVVFGGGTKLTVLGQPKAAPS |
| | PSSKSTSGGTAALGCLVKDYFPEPVTVSW | VTLFPPSSEELQANKATLVCLISDFYPGAV |
| | NSGALTSGVHTFPAVLQSSGLYSLSSVVTV | TVAWKADSSPVKAGVETTTPSKQSNNKY |
| | PSSSLGTQTYICNVNHKPSNTKVDKKV | AASSYLSLTPEQWKSHRSYSCQVTHEGST |
| | (SEQ ID NO: 27) | VEKTVAPTECS |
| | | (SEQ ID NO: 12) |
| | HC1 | HC2 |
| | QVQLVESGGGVVQPGRSLRLSCAASGFIFS | EVQLVQSGAEVKKPGESLKISCKGSGYSFS |
| | SYAMHWVRQAPGNGLEWVAFMSYDGSN | NYWIGWVRQMPGKGLEWMGIIDPSNSYT |
| | KKYADSVKGRFTISRDNSKNTLYLQMNSL | RYSPSFQGQVTISADKSISTAYLQWSSLKA |
| | RAEDTAVYYCARDRGIAAGGNYYYYGM | SDTAMYYCARWYYKPFDVWGQGTLVTV |
| | DVWGQGTTVTVSSRTVAAPSVFIFPPSDEQ | SSASTKGPSVFPLAPSSKSTSGGTAALGCL |
| | LKSGTASVVCLLNNFYPREAKVQWKVDN | VKDYFPEPVTVSWNSGALTSGVHTFPAVL |
| | ALQSGNSQESVTEQDSKDSTYSLSSTLTLS | QSSGLYSLSSVVTVPSSSLGTQTYICNVNH |
| | KADYEKHKVYACEVTHQGLSSPVTKSFNR | KPSNTKVDKKVEPKSCDKTHTCPPCPAPE |
| | GECEPKSCDKTHTCPPCPAPELLGGPSVFL | LLGGPSVFLFPPKPKDTLMISRTPEVTCVV |
| | FPPKPKDTLMISRTPEVTCVVVDVSHEDPE | VDVSHEDPEVKFNWYVDGVEVHNAKTKP |
| | VKFNWYVDGVEVHNAKTKPREEQYNSTY | REEQYNSTYRVVSVLTVLHQDWLNGKEY |
| | RVVSVLTVLHQDWLNGKEYKCKVSNKAL | KCKVSNKALPAPIEKTISKAKGQPREPQVC |
| | PAPIEKTISKAKGQPREPQVYTLPPCRDELT | TLPPSRDELTKNQVSLSCAVKGFYPSDIAV |
| | KNQVSLWCLVKGFYPSDIAVEWESNGQPE | EWESNGQPENNYKTTPPVLDSDGSFFLVS |
| | NNYKTTPPVLDSDGSFFLYSKLTVDKSRW | KLTVDKSRWQQGNVFSCSVMHEALHNHY |
| | QQGNVFSCSVMHEALHNHYTQKSLSLSPG | TQKSLSLSPGK |
| | K | (SEQ ID NO: 26) |
| | (SEQ ID NO: 28) | |
| A7 | LC1 | LC2 |
| | DIQMTQSPSSLSASVGDRVTITCRASQGIR | DIQMTQSPSSLSASVGDRVTITCRTSENIYS |
| | NYLAWYQQKPGKAPKLLIYAASTLQSGVP | YLAWYQQKPGKAPKLLIYNAKTLAEGVPS |
| | SRFSGSGSGTDFTLTISSLQPEDVATYYCQ | RFSGSGSGTDFTLTISSLQPEDFATYYCQH |
| | RYNRAPYTFGQGTKVEIKASTKGPSVFPLA | HYGIPFTFGQGTKVEIKRTVAAPSVFIFPPS |
| | PSSKSTSGGTAALGCLVKDYFPEPVTVSW | DEQLKSGTASVVCLLNNFYPREAKVQWK |
| | NSGALTSGVHTFPAVLQSSGLYSLSSVVTV | VDNALQSGNSQESVTEQDSKDSTYSLSSTL |
| | PSSSLGTQTYICNVNHKPSNTKVDKKV | TLSKADYEKHKVYACEVTHQGLSSPVTKS |
| | (SEQ ID NO: 24) | FNRGEC |
| | | (SEQ ID NO: 16) |
| | HC1 | HC2 |
| | EVQLVESGGGLVQPGRSLRLSCAASGFTF | QVQLVQSGAEVKKPGASVKVSCKASGYIF |
| | DDYAMHWVRQAPGKGLEWVSAITWNSG | ITYWMTWVRQAPGQGLEWMGQIFPASGS |
| | HIDYADSVEGRFTISRDNAKNSLYLQMNS | ADYNEKFEGRVTMTTDTSTSTAYMELRSL |
| | LRAEDTAVYYCAKVSYLSTASSLDYWGQ | RSDDTAVYYCARGGGGFAYWGQGTLVT |
| | GTLVTVSSRTVAAPSVFIFPPSDEQLKSGT | VSSASTKGPSVFPLAPSSKSTSGGTAALGC |
| | ASVVCLLNNFYPREAKVQWKVDNALQSG | LVKDYFPEPVTVSWNSGALTSGVHTFPAV |
| | NSQESVTEQDSKDSTYSLSSTLTLSKADYE | LQSSGLYSLSSVVTVPSSSLGTQTYICNVN |
| | KHKVYACEVTHQGLSSPVTKSFNRGECEP | HKPSNTKVDKKVEPKSCDKTHTCPPCPAP |
| | KSCDKTHTCPPCPAPELLGGPSVFLFPPKP | ELLGGPSVFLFPPKPKDTLMISRTPEVTCV |
| | KDTLMISRTPEVTCVVVDVSHEDPEVKFN | VVDVSHEDPEVKFNWYVDGVEVHNAKT |
| | WYVDGVEVHNAKTKPREEQYNSTYRVVS | KPREEQYNSTYRVVSVLTVLHQDWLNGK |
| | VLTVLHQDWLNGKEYKCKVSNKALPAPIE | EYKCKVSNKALPAPIEKTISKAKGQPREPQ |
| | KTISKAKGQPREPQVYTLPPCRDELTKNQ | VCTLPPSRDELTKNQVSLSCAVKGFYPSDI |
| | VSLWCLVKGFYPSDIAVEWESNGQPENNY | AVEWESNGQPENNYKTTPPVLDSDGSFFL |
| | KTTPPVLDSDGSFFLYSKLTVDKSRWQQG | VSKLTVDKSRWQQGNVFSCSVMHEALHN |
| | NVFSCSVMHEALHNHYTQKSLSLSPGK | HYTQKSLSLSPGK |
| | (SEQ ID NO: 25) | (SEQ ID NO: 29) |
| A8 | LC1 | LC2 |
| | EIVLTQSPATLSLSPGERATLSCRASQSVYS | DIQMTQSPSSLSASVGDRVTITCRTSENIYS |
| | YLAWYQQKPGQAPRLLIYDASNRATGIPA | YLAWYQQKPGKAPKLLIYNAKTLAEGVPS |
| | RFSGSGSGTDFTLTISSLEPEDFAVYYCQQ | RFSGSGSGTDFTLTISSLQPEDFATYYCQH |
| | RSNWPPFTFGPGTKVDIKASTKGPSVFPLA | HYGIPFTFGQGTKVEIKRTVAAPSVFIFPPS |
| | PSSKSTSGGTAALGCLVKDYFPEPVTVSW | DEQLKSGTASVVCLLNNFYPREAKVQWK |
| | NSGALTSGVHTFPAVLQSSGLYSLSSVVTV | VDNALQSGNSQESVTEQDSKDSTYSLSSTL |
| | PSSSLGTQTYICNVNHKPSNTKVDKKV | TLSKADYEKHKVYACEVTHQGLSSPVTKS |
| | (SEQ ID NO: 27) | FNRGEC |
| | | (SEQ ID NO: 16) |
| | HC1 | HC2 |
| | QVQLVESGGGVVQPGRSLRLSCAASGFIFS | QVQLVQSGAEVKKPGASVKVSCKASGYIF |
| | SYAMHWVRQAPGNGLEWVAFMSYDGSN | ITYWMTWVRQAPGQGLEWMGQIFPASGS |
| | KKYADSVKGRFTISRDNSKNTLYLQMNSL | ADYNEKFEGRVTMTTDTSTSTAYMELRSL |
| | RAEDTAVYYCARDRGIAAGGNYYYYGM | RSDDTAVYYCARGGGGFAYWGQGTLVT |
| | DVWGQGTTVTVSSRTVAAPSVFIFPPSDEQ | VSSASTKGPSVFPLAPSSKSTSGGTAALGC |
| | LKSGTASVVCLLNNFYPREAKVQWKVDN | LVKDYFPEPVTVSWNSGALTSGVHTFPAV |
| | ALQSGNSQESVTEQDSKDSTYSLSSTLTLS | LQSSGLYSLSSVVTVPSSSLGTQTYICNVN |
| | KADYEKHKVYACEVTHQGLSSPVTKSFNR | HKPSNTKVDKKVEPKSCDKTHTCPPCPAP |
| | GECEPKSCDKTHTCPPCPAPELLGGPSVFL | ELLGGPSVFLFPPKPKDTLMISRTPEVTCV |
| | FPPKPKDTLMISRTPEVTCVVVDVSHEDPE | VVDVSHEDPEVKFNWYVDGVEVHNAKT |
| | VKFNWYVDGVEVHNAKTKPREEQYNSTY | KPREEQYNSTYRVVSVLTVLHQDWLNGK |
| | RVVSVLTVLHQDWLNGKEYKCKVSNKAL | EYKCKVSNKALPAPIEKTISKAKGQPREPQ |
| | PAPIEKTISKAKGQPREPQVYTLPPCRDELT | VCTLPPSRDELTKNQVSLSCAVKGFYPSDI |
| | KNQVSLWCLVKGFYPSDIAVEWESNGQPE | AVEWESNGQPENNYKTTPPVLDSDGSFFL |
| | NNYKTTPPVLDSDGSFFLYSKLTVDKSRW | VSKLTVDKSRWQQGNVFSCSVMHEALHN |
| | QQGNVFSCSVMHEALHNHYTQKSLSLSPG | HYTQKSLSLSPGK |
| | K | (SEQ ID NO: 29) |
| | (SEQ ID NO: 28) | |

In some embodiments, provided herein are bispecific antibody that specifically bind to human TNFα and to human IL23p19 having LC1, HC1, LC2, and HC2, wherein the LC1, HC1, LC2, and HC2 each has at least 90%, at least 95%, at least 98%, at least 99%, or 100% sequence identity to the amino acid sequences of (1) SEQ ID NOs: 18, 19, 3, and 20, respectively; (2) SEQ ID NOs: 21, 22, 3, and 20 respectively; (3) SEQ ID NOs: 18, 19, 7 and 23, respectively; (4) SEQ ID NOs: 21, 22, 7, and 23, respectively; (5) SEQ ID NOs: 24, 25, 12, and 26, respectively; (6) SEQ ID NOs: 27, 28, 12, and 26, respectively; (7) SEQ ID NOs: 24, 25, 16, and 29, respectively; or (8) SEQ ID NOs: 27, 28, 16, and 29, respectively.

In some embodiments, provided herein are bispecific antibody that specifically bind to human TNFα and to human IL23p19 having LC1, HC1, LC2, and HC2, wherein the LC1, HC1, LC2, and HC2 each has at least 90% at least 95%, at least 98% a, at least 99% or 100% a sequence identity to the amino acid sequences of SEQ ID NOs: 18, 19, 3, and 20, respectively. In some embodiments, the LC1 has at least 90%, at least 95%, at least 98%, at least 99%, or 100% sequence identity to SEQ ID NO: 18. In some embodiments, the LC1 has the amino acid sequence of SEQ ID NO: 18. In some embodiments, the HC1 has at least 90%, at least 95%, at least 98%, at least 99%, or 100% sequence identity to SEQ ID NO: 19. In some embodiments, the HC1 has the amino acid sequence of SEQ ID NO: 19. In some embodiments, the LC2 has at least 90%, at least 95%, at least 98%, at least 99%, or 100% sequence identity to SEQ ID NO: 3. In some embodiments, the LC2 has the amino acid sequence of SEQ ID NO: 3. In some embodiments, the HC2 has at least 90%, at least 95%, at least 98%, at least 99%, or 100% sequence identity to SEQ ID NO: 20. In some embodiments, the HC2 has the amino acid sequence of SEQ ID NO: 20. In some embodiments, the LC1, HC1, LC2, and HC2 each has at least 90% sequence identity to the amino acid sequences of SEQ ID NOs: 18, 19, 3, and 20, respectively. In some embodiments, the LC1, HC1, LC2, and HC2 each has at least 95% sequence identity to the amino acid sequences of SEQ ID NOs: 18, 19, 3, and 20, respectively. In some embodiments, the LC1, HC1, LC2, and HC2 each has at least 98% sequence identity to the amino acid sequences of SEQ ID NOs: 18, 19, 3, and 20, respectively. In some embodiments, the LC1, HC1, LC2, and HC2 each has at least 99% sequence identity to the amino acid sequences of SEQ ID NOs: 18, 19, 3, and 20, respectively. In some embodiments, provided herein is the bispecific antibody designated as A1, of which the LC1, HC1, LC2, and HC2 have the amino acid sequences of SEQ ID NOs: 18, 19, 3, and 20, respectively.

In some embodiments, provided herein are bispecific antibody that specifically bind to human TNFα and to human IL23p19 having LC1, HC1, LC2, and HC2, wherein the LC1, HC1, LC2, and HC2 each has at least 90%, at least 95%, at least 98%, at least 99%, or 100% sequence identity to the amino acid sequences of SEQ ID NOs: 21, 22, 3, and 20 respectively. In some embodiments, the LC1 has at least 90%, at least 95%, at least 98%, at least 99%, or 100% sequence identity to SEQ ID NO: 21. In some embodiments, the LC1 has the amino acid sequence of SEQ ID NO: 21. In some embodiments, the HC1 has at least 90%, at least 95%, at least 98%, at least 99%, or 100% sequence identity to SEQ ID NO: 22. In some embodiments, the HC1 has the amino acid sequence of SEQ ID NO: 22. In some embodiments, the LC2 has at least 90%, at least 95%, at least 98%, at least 99%, or 100% sequence identity to SEQ ID NO: 3. In some embodiments, the LC2 has the amino acid sequence of SEQ ID NO: 3. In some embodiments, the HC2 has at least 90%, at least 95%, at least 98%, at least 99%, or 100% sequence identity to SEQ ID NO: 20. In some embodiments, the HC2 has the amino acid sequence of SEQ ID NO: 20. In some embodiments, the LC1, HC1, LC2, and HC2 each has at least 90% sequence identity to the amino acid sequences of SEQ ID NOs: 21, 22, 3, and 20, respectively. In some embodiments, the LC1, HC1, LC2, and HC2 each has at least 95% sequence identity to the amino acid sequences of SEQ ID NOs: 21, 22, 3, and 20, respectively. In some embodiments, the LC1, HC1, LC2, and HC2 each has at least 98% sequence identity to the amino acid sequences of SEQ ID NOs: 21, 22, 3, and 20, respectively. In some embodiments, the LC1, HC1, LC2, and HC2 each has at least 99% sequence identity to the amino acid sequences of SEQ ID NOs: 21, 22, 3, and 20, respectively. In some embodiments, provided herein is the bispecific antibody designated as A2, of which the LC1, HC1, LC2, and HC2 have the amino acid sequences of SEQ ID NOs: 21, 22, 3, and 20, respectively.

In some embodiments, provided herein are bispecific antibody that specifically bind to human TNFα and to human IL23p19 having LC1, HC1, LC2, and HC2, wherein the LC1, HC1, LC2, and HC2 each has at least 90%, at least 95%, at least 98%, at least 99%, or 100% sequence identity to the amino acid sequences of SEQ ID NOs: 18, 19, 7 and 23, respectively. In some embodiments, the LC1 has at least 90%, at least 95%, at least 98%, at least 99%, or 100% sequence identity to SEQ ID NO: 18. In some embodiments, the LC1 has the amino acid sequence of SEQ ID NO: 18. In some embodiments, the HC1 has at least 90%, at least 95%, at least 98%, at least 99%, or 100% sequence identity to SEQ ID NO: 19. In some embodiments, the HC1 has the amino acid sequence of SEQ ID NO: 19. In some embodiments, the LC2 has at least 90%, at least 95%, at least 98%, at least 99%, or 100% sequence identity to SEQ ID NO: 7. In some embodiments, the LC2 has the amino acid sequence of SEQ ID NO: 7. In some embodiments, the HC2 has at least 90%, at least 95%, at least 98%, at least 99%, or 100% sequence identity to SEQ ID NO: 23. In some embodiments, the HC2 has the amino acid sequence of SEQ ID NO: 23. In some embodiments, the LC1, HC1, LC2, and HC2 each has at least 90% sequence identity to the amino acid sequences of SEQ ID NOs: 18, 19, 7, and 23, respectively. In some embodiments, the LC1, HC1, LC2, and HC2 each has at least 95% sequence identity to the amino acid sequences of SEQ ID NOs: 18, 19, 7, and 23, respectively. In some embodiments, the LC1, HC1, LC2, and HC2 each has at least 98% sequence identity to the amino acid sequences of SEQ ID NOs: 18, 19, 7, and 23, respectively. In some embodiments, the LC1, HC1, LC2, and HC2 each has at least 99% sequence identity to the amino acid sequences of SEQ ID NOs: 18, 19, 7, and 23, respectively. In some embodiments, provided herein is the bispecific antibody designated as A3, of which the LC1, HC1, LC2, and HC2 have the amino acid sequences of SEQ ID NOs: 18, 19, 7, and 23, respectively.

In some embodiments, provided herein are bispecific antibody that specifically bind to human TNFα and to human IL23p19 having LC1, HC1, LC2, and HC2, wherein the LC1, HC1, LC2, and HC2 each has at least 90%, at least 95%, at least 98%, at least 99%, or 100% sequence identity to the amino acid sequences of SEQ ID NOs: 21, 22, 7, and 23, respectively. In some embodiments, the LC1 has at least 90%, at least 95%, at least 98%, at least 99%, or 100% sequence identity to SEQ ID NO: 21. In some embodiments, the LC1 has the amino acid sequence of SEQ ID NO: 21. In some embodiments, the HC1 has at least 90%, at least 95%, at least 98%, at least 99%, or 100% sequence identity to SEQ ID NO: 22. In some embodiments, the HC1 has the amino acid sequence of SEQ ID NO: 22. In some embodiments, the LC2 has at least 90%, at least 95%, at least 98%, at least 99%, or 100% sequence identity to SEQ ID NO: 7. In some embodiments, the LC2 has the amino acid sequence of SEQ ID NO: 7. In some embodiments, the HC2 has at least 90%, at least 95%, at least 98%, at least 99%, or 100% sequence identity to SEQ ID NO: 23. In some embodiments, the HC2 has the amino acid sequence of SEQ ID NO: 23. In some embodiments, the LC1, HC1, LC2, and HC2 each has at least 90% sequence identity to the amino acid sequences of SEQ ID NOs: 21, 22, 7, and 23, respectively. In some embodiments, the LC1, HC1, LC2, and HC2 each has at least 95% sequence identity to the amino acid sequences of SEQ ID NOs: 21, 22, 7, and 23, respectively. In some embodiments, the LC1, HC1, LC2, and HC2 each has at least 98% sequence identity to the amino acid sequences of SEQ ID NOs: 21, 22, 7, and 23, respectively. In some embodiments, the LC1, HC1, LC2, and HC2 each has at least 99% sequence identity to the amino acid sequences of SEQ ID NOs: 21, 22, 7, and 23, respectively. In some embodiments, provided herein is the bispecific antibody designated as A4, of which the LC1, HC1, LC2, and HC2 have the amino acid sequences of SEQ ID NOs: 21, 22, 7, and 23, respectively.

In some embodiments, provided herein are bispecific antibody that specifically bind to human TNFα and to human IL23p19 having LC1, HC1, LC2, and HC2, wherein the LC1, HC1, LC2, and HC2 each has at least 90%, at least 95%, at least 98%, at least 99%, or 100% sequence identity to the amino acid sequences of SEQ ID NOs: 24, 25, 12, and 26, respectively. In some embodiments, the LC1 has at least 90%, at least 95%, at least 98%, at least 99%, or 100% sequence identity to SEQ ID NO: 24. In some embodiments, the LC1 has the amino acid sequence of SEQ ID NO: 24. In some embodiments, the HC1 has at least 90%, at least 95%, at least 98%, at least 99%, or 100% sequence identity to SEQ ID NO: 25. In some embodiments, the HC1 has the amino acid sequence of SEQ ID NO: 25. In some embodiments, the LC2 has at least 90%, at least 95%, at least 98%, at least 99%, or 100% sequence identity to SEQ ID NO: 12. In some embodiments, the LC2 has the amino acid sequence of SEQ ID NO: 12. In some embodiments, the HC2 has at least 90%, at least 95%, at least 98%, at least 99%, or 100% sequence identity to SEQ ID NO: 26. In some embodiments, the HC2 has the amino acid sequence of SEQ ID NO: 26. In some embodiments, the LC1, HC1, LC2, and HC2 each has at least 90% sequence identity to the amino acid sequences of SEQ ID NOs: 24, 25, 12, and 26, respectively. In some embodiments, the LC1, HC1, LC2, and HC2 each has at least 95% sequence identity to the amino acid sequences of SEQ ID NOs: 24, 25, 12, and 26, respectively. In some embodiments, the LC1, HC1, LC2, and HC2 each has at least 98% sequence identity to the amino acid sequences of SEQ ID NOs: 24, 25, 12, and 26, respectively. In some embodiments, the LC1, HC1, LC2, and HC2 each has at least 99% sequence identity to the amino acid sequences of SEQ ID NOs: 24, 25, 12, and 26, respectively. In some embodiments, provided herein is the bispecific antibody designated as A5, of which the LC1, HC1, LC2, and HC2 have the amino acid sequences of SEQ ID NOs: 24, 25, 12, and 26, respectively.

In some embodiments, provided herein are bispecific antibody that specifically bind to human TNFα and to human IL23p19 having LC1, HC1, LC2, and HC2, wherein the LC1, HC1, LC2, and HC2 each has at least 90%, at least 95%, at least 98%, at least 99%, or 100% sequence identity to the amino acid sequences of SEQ ID NOs: 27, 28, 12, and 26, respectively. In some embodiments, the LC1 has at least 90%, at least 95%, at least 98%, at least 99%, or 100% sequence identity to SEQ ID NO: 27. In some embodiments, the LC1 has the amino acid sequence of SEQ ID NO: 27. In some embodiments, the HC1 has at least 90%, at least 95%, at least 98%, at least 99%, or 100% sequence identity to SEQ ID NO: 28. In some embodiments, the HC1 has the amino acid sequence of SEQ ID NO: 28. In some embodiments, the LC2 has at least 90%, at least 95%, at least 98%, at least 99%, or 100% sequence identity to SEQ ID NO: 12. In some embodiments, the LC2 has the amino acid sequence of SEQ ID NO: 12. In some embodiments, the HC2 has at least 90%, at least 95%, at least 98%, at least 99%, or 100% sequence identity to SEQ ID NO: 26. In some embodiments, the HC2 has the amino acid sequence of SEQ ID NO: 26. In some embodiments, the LC1, HC1, LC2, and HC2 each has at least 90% sequence identity to the amino acid sequences of SEQ ID NOs: 27, 28, 12, and 26, respectively. In some embodiments, the LC1, HC1, LC2, and HC2 each has at least 95% sequence identity to the amino acid sequences of SEQ ID NOs: 27, 28, 12, and 26, respectively. In some embodiments, the LC1, HC1, LC2, and HC2 each has at least 98% sequence identity to the amino acid sequences of SEQ ID NOs: 27, 28, 12, and 26, respectively. In some embodiments, the LC1, HC1, LC2, and HC2 each has at least 99% sequence identity to the amino acid sequences of SEQ ID NOs: 27, 28, 12, and 26, respectively. In some embodiments, provided herein is the bispecific antibody designated as A6, of which the LC1, HC1, LC2, and HC2 have the amino acid sequences of SEQ ID NOs: 27, 28, 12, and 26, respectively.

In some embodiments, provided herein are bispecific antibody that specifically bind to human TNFα and to human IL23p19 having LC1, HC1, LC2, and HC2, wherein LC1, HC1, LC2, and HC2 each has at least 90%, at least 95%, at least 98%, at least 99%, or 100% sequence identity to the amino acid sequences of SEQ ID NOs: 24, 25, 16, and 29, respectively. In some embodiments, the LC1 has at least 90%, at least 95%, at least 98%, at least 99%, or 100% sequence identity to SEQ ID NO: 24. In some embodiments, the LC1 has the amino acid sequence of SEQ ID NO: 24. In some embodiments, the HC1 has at least 90%, at least 95%, at least 98%, at least 99%, or 100% sequence identity to SEQ ID NO: 25. In some embodiments, the HC1 has the amino acid sequence of SEQ ID NO: 25. In some embodiments, the LC2 has at least 90%, at least 95%, at least 98%, at least 99%, or 100% sequence identity to SEQ ID NO: 16. In some embodiments, the LC2 has the amino acid sequence of SEQ ID NO: 16. In some embodiments, the HC2 has at least 90%, at least 95%, at least 98%, at least 99%, or 100% sequence identity to SEQ ID NO: 29. In some embodiments, the HC2 has the amino acid sequence of SEQ ID NO: 29. In some embodiments, the LC1, HC1, LC2, and HC2 each has at least 90% sequence identity to the amino acid sequences of SEQ ID NOs: 24, 25, 16, and 29, respectively. In some embodiments, the LC1, HC1, LC2, and HC2 each has at least 95% sequence identity to the amino acid sequences of SEQ ID NOs: 24, 25, 16, and 29, respectively. In some embodiments, the LC1, HC1, LC2, and HC2 each has at least 98% sequence identity to the amino acid sequences of SEQ ID NOs: 24, 25, 16, and 29, respectively. In some embodiments, the LC1, HC1, LC2, and HC2 each has at least 99% sequence identity to the amino acid sequences of SEQ ID NOs: 24, 25, 16, and 29, respectively. In some embodiments, provided herein is the bispecific antibody designated as A7, of which the LC1, HC1, LC2, and HC2 have the amino acid sequences of SEQ ID NOs: 24, 25, 16, and 29, respectively.

In some embodiments, provided herein are bispecific antibody that specifically bind to human TNFα and to human IL23p19 having LC1, HC1, LC2, and HC2, wherein LC1, HC1, LC2, and HC2 each has at least 90%, at least 95%, at least 98%, at least 99%, or 100% sequence identity to the amino acid sequences of SEQ ID NOs: 27, 28, 16, and 29, respectively. In some embodiments, the LC1 has at least 90%, at least 95%, at least 98%, at least 99%, or 100% sequence identity to SEQ ID NO: 27. In some embodiments, the LC1 has the amino acid sequence of SEQ ID NO: 27. In some embodiments, the HC1 has at least 90%, at least 95%, at least 98%, at least 99%, or 100% sequence identity to SEQ ID NO: 28. In some embodiments, the HC1 has the amino acid sequence of SEQ ID NO: 28. In some embodiments, the LC2 has at least 90%, at least 95%, at least 98%, at least 99%, or 100% sequence identity to SEQ ID NO: 16. In some embodiments, the LC2 has the amino acid sequence of SEQ ID NO: 16. In some embodiments, the HC2 has at least 90%, at least 95%, at least 98%, at least 99%, or 100% sequence identity to SEQ ID NO: 29. In some embodiments, the HC2 has the amino acid sequence of SEQ ID NO: 29. In some embodiments, the LC1, HC1, LC2, and HC2 each has at least 90% sequence identity to the amino acid sequences of SEQ ID NOs: 27, 28, 16, and 29, respectively. In some embodiments, the LC1, HC1, LC2, and HC2 each has at least 95% sequence identity to the amino acid sequences of SEQ ID NOs: 27, 28, 16, and 29, respectively. In some embodiments, the LC1, HC1, LC2, and HC2 each has at least 98% sequence identity to the amino acid sequences of SEQ ID NOs: 27, 28, 16, and 29, respectively. In some embodiments, the LC1, HC1, LC2, and HC2 each has at least 99% sequence identity to the amino acid sequences of SEQ ID NOs: 27, 28, 16, and 29, respectively. In some embodiments, provided herein is the bispecific antibody designated as A8, of which the LC1, HC1, LC2, and HC2 have the amino acid sequences of SEQ ID NOs: 27, 28, 16, and 29, respectively. 2.3 IgG-ScFv

In some embodiments, the bispecific antibodies provided herein that specifically bind to human TNFα and to human IL23p19 have the “IgG-ScFv” structure depicted in FIG. 1B. As shown, an scFv that comprises a VH/VL pair that specifically binds to a first antigen is linked to the CH3 domain of an IgG that specifically binds to a second antigen. In some embodiments, the N-terminus of the scFv is linked to the C-terminus of the heavy chain of the IgG. In some embodiments, the N-terminus of the scFv is linked to the C-terminus of the CH3 domain of the IgG. In some embodiments, the N-terminus of the scFv is linked to the C-terminus of the light chain of the IgG. In some embodiments, the N-terminus of the scFv is linked to the C-terminus of the VL region of the IgG. In some embodiments, the scFv has a linker that connects the VH and VL. In some embodiments of the scFv, the N-terminus of the VH is linked to the C-terminus of the VL in the scFv.

In some embodiments of the scFv, the N-terminus of the VL is linked to the C-terminus of the VH in the scFv.

In some embodiments, the scFv specifically binds to human TNFα and the IgG specifically binds to human IL23p19. In some embodiments, the scFv specifically binds to human IL23p19 and the IgG specifically binds to human TNFα.

Accordingly, provided herein are bispecific antibodies that specifically bind to human TNFα and to human IL23p19, comprising: (1) a light chain (LC) comprising a first light chain variable domain (VL1) and a CL region; and (2) a heavy chain (HC) comprising a first heavy chain variable domain (VH1), a heavy chain constant region (CH), a second light chain variable domain (VL2); and a second heavy chain variable domain (VH2); wherein the VL1/VH1 pair and VL2/VH2 pair specifically bind to human TNFα and human IL23p19, respectively, or to human IL23p19 and human TNFα, respectively. In some embodiments, the bispecific antibodies provided herein have two identical pairs of the LC and HC.

Alternatively, provided herein are bispecific antibodies that specifically bind to human TNFα and to human IL23p19, comprising: (1) a light chain (LC) comprising a first light chain variable domain (VL1), a CL region, a second light chain variable domain (VL2), and a second heavy chain variable domain (VH2); and (2) a heavy chain (HC) comprising a first heavy chain variable domain (VH1) and a heavy chain constant region (CH); wherein the VL1/VH1 pair and VL2/VH2 pair specifically bind to human TNFα and human IL23p19, respectively, or to human IL23p19 and human TNFα, respectively. In some embodiments, the bispecific antibodies provided herein have two identical pairs of the LC and HC.

Alternatively, provided herein are bispecific antibodies that specifically bind to human TNFα and to human IL23p19, comprising: (1) a light chain (LC) comprising a first light chain variable domain (VL1) and a CL region; and (2) a heavy chain (HC) comprising a first heavy chain variable domain (VH1), a heavy chain constant region (CH), a second heavy chain variable domain (VH2); and a second light chain variable domain (VL2); wherein the VL1/VH1 pair and VL2/VH2 pair specifically bind to human TNFα and human IL23p19, respectively, or to human IL23p19 and human TNFα, respectively. In some embodiments, the bispecific antibodies provided herein have two identical pairs of the LC and HC.

Alternatively, provided herein are bispecific antibodies that specifically bind to human TNFα and to human IL23p19, comprising: (1) a light chain (LC) comprising a first light chain variable domain (VL1), a CL region, a second heavy chain variable domain (VH2), and a second light chain variable domain (VL2); and (2) a heavy chain (HC) comprising a first heavy chain variable domain (VH1) and a heavy chain constant region (CH); wherein the VL1/VH1 pair and VL2/VH2 pair specifically bind to human TNFα and human IL23p19, respectively, or to human IL23p19 and human TNFα, respectively. In some embodiments, the bispecific antibodies provided herein have two identical pairs of the LC and HC.

The amino acid sequences of the CL and the CH region of the bispecific antibodies disclosed herein can be derived from any appropriate source, e.g., a constant region of an antibody such as an IgG1, IgG2, IgG3, or IgG4. In some embodiments, the constant domains and constant regions of the bispecific antibodies provided herein are derived from human IgG. In some embodiments, the constant domains and constant regions of the bispecific antibodies provided herein are derived from human IgG1. In some embodiments, the constant domains and constant regions of the bispecific antibodies provided herein are derived from human IgG2. In some embodiments, the constant domains and constant regions of the bispecific antibodies provided herein are derived from human IgG3. In some embodiments, the constant domains and constant regions of the bispecific antibodies provided herein are derived from human IgG4. In some embodiments, the amino acid sequences of the CH1, the CL region, and the Fc region (hinge, CH2 and CH3) of the bispecific antibodies disclosed herein can comprise one or more amino acid substitutions that differ from the wild type immunoglobulin, e.g., one or more amino acid substitutions in a wild type IgG1 or IgG4. Such substitutions are known in the art (see, e.g., U.S. Pat. Nos. 7,704,497, 7,083,784, 6,821,505, 8,323,962, 6,737,056, and 7,416,727).

In some embodiments, the CH region can be selected from the group consisting of a human IgG1 CH region (SEQ ID NO: 58), a human IgG2 CH region (SEQ ID NO: 80), a human IgG3 CH region (SEQ ID NO: 81), and a human IgG4 CH region (SEQ ID NO: 82). In some embodiments, the CH region is human IgG1 CH region (SEQ ID NO: 58).

In some embodiments, the CL region can be kappa CL (Cκ; SEQ ID NO: 68). In some embodiments, the CL region can be lambda CL (Ck, SEQ ID NO: 69).

In some embodiments of the bispecific antibodies provided herein, (1) the CL region is Cκ (SEQ ID NO: 68) or C (SEQ ID NO: 69); or (2) the CH region is human IgG1 CH region (SEQ ID NO: 58); or both (1) and (2).

In some embodiments of the bispecific antibodies provided herein, the scFv is linked to the CH region or the CL region via a linker. In some embodiments, the linker has the amino acid sequence of (GGGGS)n, n=1, 2, 3, 4, or 5 (SEQ ID NO:71). In some embodiments, the linker has the amino acid sequence of GGGGSGGGGSGGGGS (SEQ ID NO: 72). In some embodiments, the linker has the amino acid sequence of (EAAAK)n, n=1, 2, 3, 4, or 5 (SEQ ID NO:73).

In some embodiments of the bispecific antibodies provided herein, the VL2 and VH2 of the scFv are linked via a linker. In some embodiments, the linker has the amino acid sequence of (GGGGS)n, n=1, 2, 3, 4, or 5 (SEQ ID NO:71). In some embodiments, the linker has the amino acid sequence of GGGGSGGGGSGGGGS (SEQ ID NO: 72). In some embodiments, the linker has the amino acid sequence of (EAAAK)n, n=1, 2, 3, 4, or 5 (SEQ ID NO:73).

In some embodiments of the bispecific antibodies provided herein, the VL1/VH1 pair specifically binds to human TNFα and the VL2/VH2 pair specifically binds to human IL23p19. The VL1/VH1 pair can be any VL/VH pair that specifically binds to human TNFα. In some embodiments, the VL1 and VH1 are the VL and VH of adalimumab and have the amino acid sequences of SEQ ID NOs: 1 and 2, respectively. In some embodiments, the VL1 and VH1 are the VL and VH of golimumab and have the amino acid sequences of SEQ ID NOs: 5 and 6, respectively. The VL2/VH2 pair can be any VL/VH pair that specifically binds to human IL23p19. In some embodiments, the VL2 and VH2 are the VL and VH of guselkumab and have the amino acid sequences of SEQ ID NOs: 10 and 11, respectively. In some embodiments, the VL2 and VH2 are the VL and VH of guselkumab variant and have the amino acid sequences of SEQ ID NOs: 93 and 94, respectively. In some embodiments, the VL2 and VH2 are the VL and VH of tildrakizumab and have the amino acid sequences of SEQ ID NOs: 14 and 15, respectively.

In some embodiments of the bispecific antibodies provided herein, the VL1 and VH1 have the amino acid sequences of (1) SEQ ID NOs: 1 and 2, respectively; or (2) SEQ ID NOs: 5 and 6, respectively. In some embodiments, the VL1 and VH1 have the amino acid sequences of SEQ ID NOs: 1 and 2, respectively. In some embodiments, the VL1 and VH1 have the amino acid sequences of SEQ ID NOs: 5 and 6, respectively. In some embodiments of the bispecific antibodies provided herein, the VL2 and VH2 have the amino acid sequences of (1) SEQ ID NOs: 10 and 11, respectively; or (2) SEQ ID NOs: 14 and 15, respectively; or (3) SEQ ID NOs: 93 and 94, respectively. In some embodiments, the VL2 and VH2 have the amino acid sequences of SEQ ID NOs: 10 and 11, respectively. In some embodiments, the VL2 and VH2 have the amino acid sequences of SEQ ID NOs: 14 and 15, respectively. In some embodiments, the VL2 and VH2 have the amino acid sequences of SEQ ID NOs: 93 and 94, respectively.

In some embodiments of the bispecific antibodies provided herein, the VL1, VH1, VL2 and VH2 have the amino acid sequences of (1) SEQ ID NOs: 1, 2, 10, and 11, respectively; (2) SEQ ID NOs: 1, 2, 14, and 15, respectively; (3) SEQ ID NOs: 1, 2, 93, and 94, respectively; (4) SEQ ID NOs: 5, 6, 10, and 11, respectively; (5) SEQ ID NOs: 5, 6, 14, and 15, respectively; or (6) SEQ ID NOs: 5, 6, 93, and 94, respectively. In some embodiments, the VL1, VH1, VL2 and VH2 have the amino acid sequences of SEQ ID NOs: 1, 2, 10, and 11, respectively. In some embodiments, the VL1, VH1, VL2 and VH2 have the amino acid sequences of SEQ ID NOs: 1, 2, 14, and 15, respectively. In some embodiments, the VL1, VH1, VL2 and VH2 have the amino acid sequences of SEQ ID NOs: 1, 2, 93, and 94, respectively. In some embodiments, the VL1, VH1, VL2 and VH2 have the amino acid sequences of SEQ ID NOs: 5, 6, 10, and 11, respectively. In some embodiments, the VL1, VH1, VL2 and VH2 have the amino acid sequences of SEQ ID NOs: 5, 6, 14, and 15, respectively. In some embodiments, the VL1, VH1, VL2 and VH2 have the amino acid sequences of SEQ ID NOs: 5, 6, 93, and 94, respectively.

In some embodiments of the bispecific antibodies provided herein, the VL1/VH1 pair specifically binds to human IL23p19 and the VL2/VH2 pair specifically binds to human TNFα. The VL1/VH1 pair can be any VL/VH pair that specifically binds to human IL23p19. In some embodiments, the VL1 and VH1 are the VL and VH of guselkumab and have the amino acid sequences of SEQ ID NOs: 10 and 11, respectively. In some embodiments, the VL1 and VHI are the VL and VH of tildrakizumab and have the amino acid sequences of SEQ ID NOs: 14 and 15, respectively. The VL2/VH2 pair can be any VL/VH pair that specifically binds to human TNFα. In some embodiments, the VL2 and VH2 are the VL and VH of adalimumab and have the amino acid sequences of SEQ ID NOs: 1 and 2, respectively. In some embodiments, the VL2 and VH2 are the VL and VH of golimumab and have the amino acid sequences of SEQ ID NOs: 5 and 6, respectively.

In some embodiments of the bispecific antibodies provided herein, the VL1 and VH1 have the amino acid sequences of (1) SEQ ID NOs: 10 and 11, respectively; or (2) SEQ ID NOs: 14 and 15, respectively. In some embodiments, the VL1 and VH1 have the amino acid sequences of SEQ ID NOs: 10 and 11, respectively. In some embodiments, the VL1 and VH1 have the amino acid sequences of SEQ ID NOs: 14 and 15, respectively. In some embodiments of the bispecific antibodies provided herein, the VL2 and VH2 have the amino acid sequences of (1) SEQ ID NOs: 1 and 2, respectively; or (2) SEQ ID NOs: 5 and 6, respectively. In some embodiments, the VL2 and VH2 have the amino acid sequences of SEQ ID NOs: 1 and 2, respectively. In some embodiments, the VL2 and VH2 have the amino acid sequences of SEQ ID NOs: 5 and 6, respectively.

In some embodiments of the bispecific antibodies provided herein, the VL1, VH1, VL2 and VH2 have the amino acid sequences of (1) SEQ ID NOs: 10, 11, 1, and 2, respectively; (2) SEQ ID NOs: 10, 11, 5, and 6, respectively; (3) SEQ ID NOs: 14, 15, 1, and 2, respectively, or (4) SEQ ID NOs: 14, 15, 5, and 6, respectively. In some embodiments, the VL1, VH1, VL2 and VH2 have the amino acid sequences of SEQ ID NOs: 10, 11, 1, and 2, respectively. In some embodiments, the VL1, VH1, VL2 and VH2 have the amino acid sequences of SEQ ID NOs: 10, 11, 5, and 6, respectively.

In some embodiments, the VL1, VH1, VL2 and VH2 have the amino acid sequences of SEQ ID NOs: 14, 15, 1, and 2, respectively. In some embodiments, the VL1, VH1, VL2 and VH2 have the amino acid sequences of SEQ ID NOs: 14, 15, 5, and 6, respectively. A list of exemplary LC and HC of bispecific antibodies is provided below as Table 4B.

TABLE 4B
Exemplified Bispecific Antibodies to human TNFα and human IL23p19

| | | |
|---|---|---|
| B1 | LC | HC |
| | DIQMTQSPSSLSASVGD | EVQLVESGGGLVQPGRSLRLSCAASGFTFDDYAMHWVRQAPGKG |
| | RVTITCRASQGIRNYLA | LEWVSAITWNSGHIDYADSVEGRFTISRDNAKNSLYLQMNSLRAE |
| | WYQQKPGKAPKLLIYA | DTAVYYCAKVSYLSTASSLDYWGQGTLVTVSSASTKGPSVFPLAP |
| | ASTLQSGVPSRFSGSGS | SSKSTSGGTAALGCLVKDYFPEPVTVSWNSGALTSGVHTFPAVLQ |
| | GTDFTLTISSLQPEDVA | SSGLYSLSSVVTVPSSSLGTQTYICNVNHKPSNTKVDKKVEPKSCD |
| | TYYCQRYNRAPYTFGQ | KTHTCPPCPAPELLGGPSVFLFPPKPKDTLMISRTPEVTCVVVDVSH |
| | GTKVEIKRTVAAPSVFI | EDPEVKFNWYVDGVEVHNAKTKPREEQYNSTYRVVSVLTVLHQD |
| | FPPSDEQLKSGTASVVC | WLNGKEYKCKVSNKALPAPIEKTISKAKGQPREPQVYTLPPSRDEL |
| | LLNNFYPREAKVQWK | TKNQVSLTCLVKGFYPSDIAVEWESNGQPENNYKTTPPVLDSDGSF |
| | VDNALQSGNSQESVTE | FLYSKLTVDKSRWQQGNVFSCSVMHEALHNHYTQKSLSLSPGKG |
| | QDSKDSTYSLSSTLTLS | GGGSGGGGSGGGGSQSVLTQPPSVSGAPGQRVTISCTGSSSNIGSG |
| | KADYEKHKVYACEVT | YDVHWYQQLPGTAPKLLIYGNSKRPSGVPDRFSGSKSGTSASLAIT |
| | HQGLSSPVTKSFNRGE | GLQSEDEADYYCASWTDGLSLVVFGGGTKLTVLGGGGSGGGGSG |
| | C | GGGSEVQLVQSGAEVKKPGESLKISCKGSGYSFSNYWIGWVRQMP |
| | (SEQ ID NO: 3) | GKGLEWMGIIDPSNSYTRYSPSFQGQVTISADKSISTAYLQWSSLK |
| | | ASDTAMYYCARWYYKPFDVWGQGTLVTVSS |
| | | (SEQ ID NO: 30) |
| B2 | LC | HC |
| | DIQMTQSPSSLSASVGD | EVQLVESGGGLVQPGRSLRLSCAASGFTFDDYAMHWVRQAPGKG |
| | RVTITCRASQGIRNYLA | LEWVSAITWNSGHIDYADSVEGRFTISRDNAKNSLYLQMNSLRAE |
| | WYQQKPGKAPKLLIYA | DTAVYYCAKVSYLSTASSLDYWGQGTLVTVSSASTKGPSVFPLAP |
| | ASTLQSGVPSRFSGSGS | SSKSTSGGTAALGCLVKDYFPEPVTVSWNSGALTSGVHTFPAVLQ |
| | GTDFTLTISSLQPEDVA | SSGLYSLSSVVTVPSSSLGTQTYICNVNHKPSNTKVDKKVEPKSCD |
| | TYYCQRYNRAPYTFGQ | KTHTCPPCPAPELLGGPSVFLFPPKPKDTLMISRTPEVTCVVVDVSH |
| | GTKVEIKRTVAAPSVFI | EDPEVKFNWYVDGVEVHNAKTKPREEQYNSTYRVVSVLTVLHQD |
| | FPPSDEQLKSGTASVVC | WLNGKEYKCKVSNKALPAPIEKTISKAKGQPREPQVYTLPPSRDEL |
| | LLNNFYPREAKVQWK | TKNQVSLTCLVKGFYPSDIAVEWESNGQPENNYKTTPPVLDSDGSF |
| | VDNALQSGNSQESVTE | FLYSKLTVDKSRWQQGNVFSCSVMHEALHNHYTQKSLSLSPGKG |
| | QDSKDSTYSLSSTLTLS | GGGSGGGGSGGGGSDIQMTQSPSSLSASVGDRVTITCRTSENIYSY |
| | KADYEKHKVYACEVT | LAWYQQKPGKAPKLLIYNAKTLAEGVPSRFSGSGSGTDFTLTISSL |
| | HQGLSSPVTKSFNRGE | QPEDFATYYCQHHYGIPFTFGQGTKVEIKGGGGSGGGGSGGGGSQ |
| | C (SEQ ID NO: 3) | VQLVQSGAEVKKPGASVKVSCKASGYIFITYWMTWVRQAPGQGL |
| | | EWMGQIFPASGSADYNEKFEGRVTMTTDTSTSTAYMELRSLRSDD |
| | | TAVYYCARGGGGFAYWGQGTLVTVSS |
| | | (SEQ ID NO: 31) |
| B3 | LC | HC |
| | EIVLTQSPATLSLSPGER | QVQLVESGGGVVQPGRSLRLSCAASGFIFSSYAMHWVRQAPGNGL |
| | ATLSCRASQSVYSYLA | EWVAFMSYDGSNKKYADSVKGRFTISRDNSKNTLYLQMNSLRAE |
| | WYQQKPGQAPRLLIYD | DTAVYYCARDRGIAAGGNYYYYGMDVWGQGTTVTVSSASTKGP |
| | ASNRATGIPARFSGSGS | SVFPLAPSSKSTSGGTAALGCLVKDYFPEPVTVSWNSGALTSGVHT |
| | GTDFTLTISSLEPEDFA | FPAVLQSSGLYSLSSVVTVPSSSLGTQTYICNVNHKPSNTKVDKKV |
| | VYYCQQRSNWPPFTFG | EPKSCDKTHTCPPCPAPELLGGPSVFLFPPKPKDTLMISRTPEVTCV |
| | PGTKVDIKRTVAAPSVF | VVDVSHEDPEVKFNWYVDGVEVHNAKTKPREEQYNSTYRVVSVL |
| | IFPPSDEQLKSGTASVV | TVLHQDWLNGKEYKCKVSNKALPAPIEKTISKAKGQPREPQVYTL |
| | CLLNNFYPREAKVQW | PPSRDELTKNQVSLTCLVKGFYPSDIAVEWESNGQPENNYKTTPPV |
| | KVDNALQSGNSQESVT | LDSDGSFFLYSKLTVDKSRWQQGNVFSCSVMHEALHNHYTQKSLS |
| | EQDSKDSTYSLSSTLTL | LSPGKGGGGSGGGGSGGGGSQSVLTQPPSVSGAPGQRVTISCTGSS |
| | SKADYEKHKVYACEV | SNIGSGYDVHWYQQLPGTAPKLLIYGNSKRPSGVPDRFSGSKSGTS |
| | THQGLSSPVTKSFNRGE | ASLAITGLQSEDEADYYCASWTDGLSLVVFGGGTKLTVLGGGGSG |
| | C | GGGSGGGGSEVQLVQSGAEVKKPGESLKISCKGSGYSFSNYWIGW |
| | (SEQ ID NO: 7) | VRQMPGKGLEWMGIIDPSNSYTRYSPSFQGQVTISADKSISTAYLQ |
| | | WSSLKASDTAMYYCARWYYKPFDVWGQGTLVTVSS |
| | | (SEQ ID NO: 32) |
| B3-1 | LC | HC |
| | EIVLTQSPATLSLSPGER | QVQLVESGGGVVQPGRSLRLSCAASGFIFSSYAMHWVRQAPGNGL |
| | ATLSCRASQSVYSYLA | EWVAFMSYDGSNKKYADSVKGRFTISRDNSKNTLYLQMNSLRAE |
| | WYQQKPGQAPRLLIYD | DTAVYYCARDRGIAAGGNYYYYGMDVWGQGTTVTVSSASTKGP |
| | ASNRATGIPARFSGSGS | SVFPLAPSSKSTSGGTAALGCLVKDYFPEPVTVSWNSGALTSGVHT |
| | GTDFTLTISSLEPEDFA | FPAVLQSSGLYSLSSVVTVPSSSLGTQTYICNVNHKPSNTKVDKKV |
| | VYYCQQRSNWPPFTFG | EPKSCDKTHTCPPCPAPELLGGPSVFLFPPKPKDTLMISRTPEVTCV |
| | PGTKVDIKRTVAAPSVE | VVDVSHEDPEVKFNWYVDGVEVHNAKTKPREEQYNSTYRVVSVL |
| | IFPPSDEQLKSGTASVV | TVLHQDWLNGKEYKCKVSNKALPAPIEKTISKAKGQPREPQVYTL |
| | CLLNNFYPREAKVQW | PPSRDELTKNQVSLTCLVKGFYPSDIAVEWESNGQPENNYKTTPPV |
| | KVDNALQSGNSQESVT | LDSDGSFFLYSKLTVDKSRWQQGNVFSCSVMHEALHNHYTQKSLS |
| | EQDSKDSTYSLSSTLTL | LSPGKGGGGSGGGGSGGGGSQSVLTQPPSVSGAPGQRVTISCTGSS |
| | SKADYEKHKVYACEV | SNIGSGYDVHWYQQLPGTAPKLLIYGNSKRPSGVPDRFSGSKSGTS |
| | THQGLSSPVTKSFNRGE | ASLAITGLQSEDEADYYCASWTDGLSLVVFGCGTKLTVLGGGGSG |
| | C | GGGSGGGGSEVQLVQSGAEVKKPGESLKISCKGSGYSFSNYWIGW |
| | (SEQ ID NO: 7) | VRQMPGKCLEWMGIIDPSNSYTRYSPSFQGQVTISADKSISTAYLQ |
| | | WSSLKASDTAMYYCARWYYKPFDVWGQGTLVTVSS |
| | | (SEQ ID NO: 92) |
| B4 | LC | HC |
| | EIVLTQSPATLSLSPGER | QVQLVESGGGVVQPGRSLRLSCAASGFIFSSYAMHWVRQAPGNGL |
| | ATLSCRASQSVYSYLA | EWVAFMSYDGSNKKYADSVKGRFTISRDNSKNTLYLQMNSLRAE |
| | WYQQKPGQAPRLLIYD | DTAVYYCARDRGIAAGGNYYYYGMDVWGQGTTVTVSSASTKGP |
| | ASNRATGIPARFSGSGS | SVFPLAPSSKSTSGGTAALGCLVKDYFPEPVTVSWNSGALTSGVHT |
| | GTDFTLTISSLEPEDFA | FPAVLQSSGLYSLSSVVTVPSSSLGTQTYICNVNHKPSNTKVDKKV |
| | VYYCQQRSNWPPFTFG | EPKSCDKTHTCPPCPAPELLGGPSVFLFPPKPKDTLMISRTPEVTCV |
| | PGTKVDIKRTVAAPSVF | VVDVSHEDPEVKFNWYVDGVEVHNAKTKPREEQYNSTYRVVSVL |
| | IFPPSDEQLKSGTASVV | TVLHQDWLNGKEYKCKVSNKALPAPIEKTISKAKGQPREPQVYTL |
| | CLLNNFYPREAKVQW | PPSRDELTKNQVSLTCLVKGFYPSDIAVEWESNGQPENNYKTTPPV |
| | KVDNALQSGNSQESVT | LDSDGSFFLYSKLTVDKSRWQQGNVFSCSVMHEALHNHYTQKSLS |
| | EQDSKDSTYSLSSTLTL | LSPGKGGGGSGGGGSGGGGSDIQMTQSPSSLSASVGDRVTITCRTS |
| | SKADYEKHKVYACEV | ENIYSYLAWYQQKPGKAPKLLIYNAKTLAEGVPSRFSGSGSGTDFT |
| | THQGLSSPVTKSFNRGE | LTISSLQPEDFATYYCQHHYGIPFTFGQGTKVEIKGGGGSGGGGSG |
| | C | GGGSQVQLVQSGAEVKKPGASVKVSCKASGYIFITYWMTWVRQA |
| | (SEQ ID NO: 7) | PGQGLEWMGQIFPASGSADYNEKFEGRVTMTTDTSTSTAYMELRS |
| | | LRSDDTAVYYCARGGGGFAYWGQGTLVTVSS |
| | | (SEQ ID NO: 33) |
| B5 | LC | HC |
| | QSVLTQPPSVSGAPGQ | EVQLVQSGAEVKKPGESLKISCKGSGYSFSNYWIGWVRQMPGKGL |
| | RVTISCTGSSSNIGSGY | EWMGIIDPSNSYTRYSPSFQGQVTISADKSISTAYLQWSSLKASDTA |
| | DVHWYQQLPGTAPKL | MYYCARWYYKPFDVWGQGTLVTVSSASTKGPSVFPLAPSSKSTSG |
| | LIYGNSKRPSGVPDRFS | GTAALGCLVKDYFPEPVTVSWNSGALTSGVHTFPAVLQSSGLYSL |
| | GSKSGTSASLAITGLQS | SSVVTVPSSSLGTQTYICNVNHKPSNTKVDKKVEPKSCDKTHTCPP |
| | EDEADYYCASWTDGLS | CPAPELLGGPSVFLFPPKPKDTLMISRTPEVTCVVVDVSHEDPEVKF |
| | LVVFGGGTKLTVLGQP | NWYVDGVEVHNAKTKPREEQYNSTYRVVSVLTVLHQDWLNGKE |
| | KAAPSVTLFPPSSEELQ | YKCKVSNKALPAPIEKTISKAKGQPREPQVYTLPPSRDELTKNQVS |
| | ANKATLVCLISDFYPG | LTCLVKGFYPSDIAVEWESNGQPENNYKTTPPVLDSDGSFFLYSKL |
| | AVTVAWKADSSPVKA | TVDKSRWQQGNVFSCSVMHEALHNHYTQKSLSLSPGKGGGGSGG |
| | GVETTTPSKQSNNKYA | GGSGGGGSDIQMTQSPSSLSASVGDRVTITCRASQGIRNYLAWYQ |
| | ASSYLSLTPEQWKSHR | QKPGKAPKLLIYAASTLQSGVPSRFSGSGSGTDFTLTISSLQPEDVA |
| | SYSCQVTHEGSTVEKT | TYYCQRYNRAPYTFGQGTKVEIKGGGGSGGGGSGGGGSEVQLVE |
| | VAPTECS | SGGGLVQPGRSLRLSCAASGFTFDDYAMHWVRQAPGKGLEWVSA |
| | (SEQ ID NO: 12) | ITWNSGHIDYADSVEGRFTISRDNAKNSLYLQMNSLRAEDTAVYY |
| | | CAKVSYLSTASSLDYWGQGTLVTVSS |
| | | (SEQ ID NO: 34) |
| B6 | LC | HC |
| | QSVLTQPPSVSGAPGQ | EVQLVQSGAEVKKPGESLKISCKGSGYSFSNYWIGWVRQMPGKGL |
| | RVTISCTGSSSNIGSGY | EWMGIIDPSNSYTRYSPSFQGQVTISADKSISTAYLQWSSLKASDTA |
| | DVHWYQQLPGTAPKL | MYYCARWYYKPFDVWGQGTLVTVSSASTKGPSVFPLAPSSKSTSG |
| | LIYGNSKRPSGVPDRFS | GTAALGCLVKDYFPEPVTVSWNSGALTSGVHTFPAVLQSSGLYSL |
| | GSKSGTSASLAITGLQS | SSVVTVPSSSLGTQTYICNVNHKPSNTKVDKKVEPKSCDKTHTCPP |
| | EDEADYYCASWTDGLS | CPAPELLGGPSVFLFPPKPKDTLMISRTPEVTCVVVDVSHEDPEVKF |
| | LVVFGGGTKLTVLGQP | NWYVDGVEVHNAKTKPREEQYNSTYRVVSVLTVLHQDWLNGKE |
| | KAAPSVTLFPPSSEELQ | YKCKVSNKALPAPIEKTISKAKGQPREPQVYTLPPSRDELTKNQVS |
| | ANKATLVCLISDFYPG | LTCLVKGFYPSDIAVEWESNGQPENNYKTTPPVLDSDGSFFLYSKL |
| | AVTVAWKADSSPVKA | TVDKSRWQQGNVFSCSVMHEALHNHYTQKSLSLSPGKGGGGSGG |
| | GVETTTPSKQSNNKYA | GGSGGGGSEIVLTQSPATLSLSPGERATLSCRASQSVYSYLAWYQQ |
| | ASSYLSLTPEQWKSHR | KPGQAPRLLIYDASNRATGIPARFSGSGSGTDFTLTISSLEPEDFAVY |
| | SYSCQVTHEGSTVEKT | YCQQRSNWPPFTFGPGTKVDIKGGGGSGGGGSGGGGSQVQLVES |
| | VAPTECS | GGGVVQPGRSLRLSCAASGFIFSSYAMHWVRQAPGNGLEWVAFM |
| | (SEQ ID NO: 12) | SYDGSNKKYADSVKGRFTISRDNSKNTLYLQMNSLRAEDTAVYY |
| | | CARDRGIAAGGNYYYYGMDVWGQGTTVTVSS |
| | | (SEQ ID NO: 35) |
| B7 | LC | HC |
| | DIQMTQSPSSLSASVGD | QVQLVQSGAEVKKPGASVKVSCKASGYIFITYWMTWVRQAPGQG |
| | RVTITCRTSENIYSYLA | LEWMGQIFPASGSADYNEKFEGRVTMTTDTSTSTAYMELRSLRSD |
| | WYQQKPGKAPKLLIYN | DTAVYYCARGGGGFAYWGQGTLVTVSSASTKGPSVFPLAPSSKST |
| | AKTLAEGVPSRFSGSGS | SGGTAALGCLVKDYFPEPVTVSWNSGALTSGVHTFPAVLQSSGLY |
| | GTDFTLTISSLQPEDFA | SLSSVVTVPSSSLGTQTYICNVNHKPSNTKVDKKVEPKSCDKTHTC |
| | TYYCQHHYGIPFTFGQ | PPCPAPELLGGPSVFLFPPKPKDTLMISRTPEVTCVVVDVSHEDPEV |
| | GTKVEIKRTVAAPSVFI | KFNWYVDGVEVHNAKTKPREEQYNSTYRVVSVLTVLHQDWLNG |
| | FPPSDEQLKSGTASVVC | KEYKCKVSNKALPAPIEKTISKAKGQPREPQVYTLPPSRDELTKNQ |
| | LLNNFYPREAKVQWK | VSLTCLVKGFYPSDIAVEWESNGQPENNYKTTPPVLDSDGSFFLYS |
| | VDNALQSGNSQESVTE | KLTVDKSRWQQGNVFSCSVMHEALHNHYTQKSLSLSPGKGGGGS |
| | QDSKDSTYSLSSTLTLS | GGGGSGGGGSDIQMTQSPSSLSASVGDRVTITCRASQGIRNYLAW |
| | KADYEKHKVYACEVT | YQQKPGKAPKLLIYAASTLQSGVPSRFSGSGSGTDFTLTISSLQPED |
| | HQGLSSPVTKSFNRGE | VATYYCQRYNRAPYTFGQGTKVEIKGGGGSGGGGSGGGGSEVQL |
| | C | VESGGGLVQPGRSLRLSCAASGFTFDDYAMHWVRQAPGKGLEWV |
| | (SEQ ID NO: 16) | SAITWNSGHIDYADSVEGRFTISRDNAKNSLYLQMNSLRAEDTAV |
| | | YYCAKVSYLSTASSLDYWGQGTLVTVSS |
| | | (SEQ ID NO: 36) |
| B8 | LC | HC |
| | DIQMTQSPSSLSASVGD | QVQLVQSGAEVKKPGASVKVSCKASGYIFITYWMTWVRQAPGQG |
| | RVTITCRTSENIYSYLA | LEWMGQIFPASGSADYNEKFEGRVTMTTDTSTSTAYMELRSLRSD |
| | WYQQKPGKAPKLLIYN | DTAVYYCARGGGGFAYWGQGTLVTVSSASTKGPSVFPLAPSSKST |
| | AKTLAEGVPSRFSGSGS | SGGTAALGCLVKDYFPEPVTVSWNSGALTSGVHTFPAVLQSSGLY |
| | GTDFTLTISSLQPEDFA | SLSSVVTVPSSSLGTQTYICNVNHKPSNTKVDKKVEPKSCDKTHTC |
| | TYYCQHHYGIPFTFGQ | PPCPAPELLGGPSVFLFPPKPKDTLMISRTPEVTCVVVDVSHEDPEV |
| | GTKVEIKRTVAAPSVFI | KFNWYVDGVEVHNAKTKPREEQYNSTYRVVSVLTVLHQDWLNG |
| | FPPSDEQLKSGTASVVC | KEYKCKVSNKALPAPIEKTISKAKGQPREPQVYTLPPSRDELTKNQ |
| | LLNNFYPREAKVQWK | VSLTCLVKGFYPSDIAVEWESNGQPENNYKTTPPVLDSDGSFFLYS |
| | VDNALQSGNSQESVTE | KLTVDKSRWQQGNVFSCSVMHEALHNHYTQKSLSLSPGKGGGGS |
| | QDSKDSTYSLSSTLTLS | GGGGSGGGGSEIVLTQSPATLSLSPGERATLSCRASQSVYSYLAWY |
| | KADYEKHKVYACEVT | QQKPGQAPRLLIYDASNRATGIPARFSGSGSGTDFTLTISSLEPEDFA |
| | HQGLSSPVTKSFNRGE | VYYCQQRSNWPPFTFGPGTKVDIKGGGGSGGGGSGGGGSQVQLV |
| | C | ESGGGVVQPGRSLRLSCAASGFIFSSYAMHWVRQAPGNGLEWVA |
| | (SEQ ID NO: 16) | FMSYDGSNKKYADSVKGRFTISRDNSKNTLYLQMNSLRAEDTAV |
| | | YYCARDRGIAAGGNYYYYGMDVWGQGTTVTVSS |
| | | (SEQ ID NO: 37) |

In some embodiments, provided herein are bispecific antibody that specifically bind to human TNFα and to human IL23p19 having LC and HC, wherein the LC and HC each has at least 9000, at least 9500, at least 98%, at least 99%, or 10000 sequence identity to the amino acid sequences of (1) SEQ ID NOs: 3 and 30, respectively; (2) SEQ ID NOs: 3 and 31 respectively; (3) SEQ ID NOs: 7 and 32, respectively; (4) SEQ ID NOs: 7 and 33, respectively; (5) SEQ ID NOs: 12 and 34, respectively; (6) SEQ ID NOs: 12 and 35, respectively; (7) SEQ ID NOs: 16 and 36, respectively; (8) SEQ ID NOs: 16 and 37, respectively; or (9) SEQ ID NOs: 7 and 92, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs: 3 and 30, respectively. In some embodiments, the LC has at least 90%, at least 95%, at least 98%, at least 99%, or 100% sequence identity to SEQ ID NO: 3. In some embodiments, the LC has the amino acid sequence of SEQ ID NO: 3. In some embodiments, the HC has at least 90%, at least 95%, at least 98%, at least 99%, or 100% sequence identity to SEQ ID NO: 30. In some embodiments, the HC has the amino acid sequence of SEQ ID NO: 30. In some embodiments, the LC and HC each has at least 90% sequence identity to the amino acid sequences of SEQ ID NOs: 3 and 30, respectively. In some embodiments, the LC and HC each has at least 95% sequence identity to the amino acid sequences of SEQ ID NOs: 3 and 30, respectively. In some embodiments, the LC and HC each has at least 98% sequence identity to the amino acid sequences of SEQ ID NOs: 3 and 30, respectively. In some embodiments, the LC and HC each has at least 99% sequence identity to the amino acid sequences of SEQ ID NOs: 3 and 30, respectively. In some embodiments, provided herein is the bispecific antibody designated as B1, of which the LC and HC have the amino acid sequences of SEQ ID NOs: 3 and 30, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:3 and 31, respectively. In some embodiments, the LC has at least 90%, at least 95%, at least 98%, at least 99%, or 100% sequence identity to SEQ ID NO: 3. In some embodiments, the LC has the amino acid sequence of SEQ ID NO: 3. In some embodiments, the HC has at least 90%, at least 95%, at least 98%, at least 99%, or 100% sequence identity to SEQ ID NO: 31. In some embodiments, the HC has the amino acid sequence of SEQ ID NO: 31. In some embodiments, the LC and HC each has at least 90% sequence identity to the amino acid sequences of SEQ ID NOs: 3 and 31, respectively. In some embodiments, the LC and HC each has at least 95% sequence identity to the amino acid sequences of SEQ ID NOs: 3 and 31, respectively. In some embodiments, the LC and HC each has at least 98% sequence identity to the amino acid sequences of SEQ ID NOs: 3 and 31, respectively. In some embodiments, the LC and HC each has at least 99% sequence identity to the amino acid sequences of SEQ ID NOs: 3 and 31, respectively. In some embodiments, provided herein is the bispecific antibody designated as B2, of which the LC and HC have the amino acid sequences of SEQ ID NOs: 3 and 31, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:7 and 32, respectively. In some embodiments, the LC has at least 90%, at least 95%, at least 98%, at least 99%, or 100% sequence identity to SEQ ID NO: 7. In some embodiments, the LC has the amino acid sequence of SEQ ID NO: 7. In some embodiments, the HC has at least 90%, at least 95%, at least 98%, at least 99%, or 100% sequence identity to SEQ ID NO: 32. In some embodiments, the HC has the amino acid sequence of SEQ ID NO: 32. In some embodiments, the LC and HC each has at least 90% sequence identity to the amino acid sequences of SEQ ID NOs: 7 and 32, respectively. In some embodiments, the LC and HC each has at least 95% sequence identity to the amino acid sequences of SEQ ID NOs: 7 and 32, respectively. In some embodiments, the LC and HC each has at least 98% sequence identity to the amino acid sequences of SEQ ID NOs: 7 and 32, respectively. In some embodiments, the LC and HC each has at least 99% sequence identity to the amino acid sequences of SEQ ID NOs: 7 and 32, respectively. In some embodiments, provided herein is the bispecific antibody designated as B3, of which the LC and HC have the amino acid sequences of SEQ ID NOs: 7 and 32, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:7 and 92, respectively. In some embodiments, the LC has at least 90%, at least 95%, at least 98%, at least 99%, or 100% sequence identity to SEQ ID NO: 7. In some embodiments, the LC has the amino acid sequence of SEQ ID NO: 7. In some embodiments, the HC has at least 90%, at least 95%, at least 98%, at least 99%, or 100% sequence identity to SEQ ID NO: 92. In some embodiments, the HC has the amino acid sequence of SEQ ID NO: 92. In some embodiments, the LC and HC each has at least 90% sequence identity to the amino acid sequences of SEQ ID NOs: 7 and 92, respectively. In some embodiments, the LC and HC each has at least 95% sequence identity to the amino acid sequences of SEQ ID NOs: 7 and 92, respectively. In some embodiments, the LC and HC each has at least 98% sequence identity to the amino acid sequences of SEQ ID NOs: 7 and 92, respectively. In some embodiments, the LC and HC each has at least 99% sequence identity to the amino acid sequences of SEQ ID NOs: 7 and 92, respectively. In some embodiments, provided herein is the bispecific antibody designated as B3-1, of which the LC and HC have the amino acid sequences of SEQ ID NOs: 7 and 92, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:7 and 33, respectively. In some embodiments, the LC has at least 90%, at least 95%, at least 98%, at least 99%, or 100% sequence identity to SEQ ID NO: 7. In some embodiments, the LC has the amino acid sequence of SEQ ID NO: 7. In some embodiments, the HC has at least 90%, at least 95%, at least 98%, at least 99%, or 100% sequence identity to SEQ ID NO: 33. In some embodiments, the HC has the amino acid sequence of SEQ ID NO: 33. In some embodiments, the LC and HC each has at least 90% sequence identity to the amino acid sequences of SEQ ID NOs: 7 and 33, respectively. In some embodiments, the LC and HC each has at least 95% sequence identity to the amino acid sequences of SEQ ID NOs: 7 and 33, respectively. In some embodiments, the LC and HC each has at least 98% sequence identity to the amino acid sequences of SEQ ID NOs: 7 and 33, respectively. In some embodiments, the LC and HC each has at least 99% sequence identity to the amino acid sequences of SEQ ID NOs: 7 and 33, respectively. In some embodiments, provided herein is the bispecific antibody designated as B4, of which the LC and HC have the amino acid sequences of SEQ ID NOs: 7 and 33, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:12 and 34, respectively. In some embodiments, the LC has at least 90%, at least 95%, at least 98%, at least 99%, or 100% sequence identity to SEQ ID NO: 12. In some embodiments, the LC has the amino acid sequence of SEQ ID NO: 12. In some embodiments, the HC has at least 90%, at least 95%, at least 98%, at least 99%, or 100% sequence identity to SEQ ID NO: 34. In some embodiments, the HC has the amino acid sequence of SEQ ID NO: 34. In some embodiments, the LC and HC each has at least 90% sequence identity to the amino acid sequences of SEQ ID NOs: 12 and 34, respectively. In some embodiments, the LC and HC each has at least 95% sequence identity to the amino acid sequences of SEQ ID NOs: 12 and 34, respectively. In some embodiments, the LC and HC each has at least 98% sequence identity to the amino acid sequences of SEQ ID NOs: 12 and 34, respectively. In some embodiments, the LC and HC each has at least 99% sequence identity to the amino acid sequences of SEQ ID NOs: 12 and 34, respectively. In some embodiments, provided herein is the bispecific antibody designated as B5, of which the LC and HC have the amino acid sequences of SEQ ID NOs: 12 and 34, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:12 and 35, respectively. In some embodiments, the LC has at least 90%, at least 95%, at least 98%, at least 99%, or 100% sequence identity to SEQ ID NO: 12. In some embodiments, the LC has the amino acid sequence of SEQ ID NO: 12. In some embodiments, the HC has at least 90%, at least 95%, at least 98%, at least 99%, or 100% sequence identity to SEQ ID NO: 35. In some embodiments, the HC has the amino acid sequence of SEQ ID NO: 35. In some embodiments, the LC and HC each has at least 90% sequence identity to the amino acid sequences of SEQ ID NOs: 12 and 35, respectively. In some embodiments, the LC and HC each has at least 95% sequence identity to the amino acid sequences of SEQ ID NOs: 12 and 35, respectively. In some embodiments, the LC and HC each has at least 98% sequence identity to the amino acid sequences of SEQ ID NOs: 12 and 35, respectively. In some embodiments, the LC and HC each has at least 99% sequence identity to the amino acid sequences of SEQ ID NOs: 12 and 35, respectively. In some embodiments, provided herein is the bispecific antibody designated as B6, of which the LC and HC have the amino acid sequences of SEQ ID NOs: 12 and 35, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:16 and 36, respectively. In some embodiments, the LC has at least 90%, at least 95%, at least 98%, at least 99%, or 100% sequence identity to SEQ ID NO: 16. In some embodiments, the LC has the amino acid sequence of SEQ ID NO: 16. In some embodiments, the HC has at least 90%, at least 95%, at least 98%, at least 99%, or 100% sequence identity to SEQ ID NO: 36. In some embodiments, the HC has the amino acid sequence of SEQ ID NO: 36. In some embodiments, the LC and HC each has at least 90% sequence identity to the amino acid sequences of SEQ ID NOs: 16 and 36, respectively. In some embodiments, the LC and HC each has at least 95% sequence identity to the amino acid sequences of SEQ ID NOs: 16 and 36, respectively. In some embodiments, the LC and HC each has at least 98% sequence identity to the amino acid sequences of SEQ ID NOs: 16 and 36, respectively. In some embodiments, the LC and HC each has at least 99% sequence identity to the amino acid sequences of SEQ ID NOs: 16 and 36, respectively. In some embodiments, provided herein is the bispecific antibody designated as B7, of which the LC and HC have the amino acid sequences of SEQ ID NOs: 16 and 36, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:16 and 37, respectively. In some embodiments, the LC has at least 90%, at least 95%, at least 98%, at least 99%, or 100% sequence identity to SEQ ID NO: 16. In some embodiments, the LC has the amino acid sequence of SEQ ID NO: 16. In some embodiments, the HC has at least 90%, at least 95%, at least 98%, at least 99%, or 100% sequence identity to SEQ ID NO: 37. In some embodiments, the HC has the amino acid sequence of SEQ ID NO: 37. In some embodiments, the LC and HC each has at least 90% sequence identity to the amino acid sequences of SEQ ID NOs: 16 and 37, respectively. In some embodiments, the LC and HC each has at least 95% sequence identity to the amino acid sequences of SEQ ID NOs: 16 and 37, respectively. In some embodiments, the LC and HC each has at least 98% sequence identity to the amino acid sequences of SEQ ID NOs: 16 and 37, respectively. In some embodiments, the LC and HC each has at least 99% sequence identity to the amino acid sequences of SEQ ID NOs: 16 and 37, respectively. In some embodiments, provided herein is the bispecific antibody designated as B8, of which the LC and HC have the amino acid sequences of SEQ ID NOs: 16 and 37, respectively. 2.4 DVD-Ig

In some embodiments, the bispecific antibodies provided herein that specifically bind to human TNFα and to human IL23p19 have the “dual variable domain-immunoglobulin” or “DVD-Ig” structure depicted in FIG. 1C. The DVD-Ig is a symmetrical structure with four antigen binding sites that can target two different targets at the same time. The DVD-Ig structure contains the Fc region, and each antibody arm uses flexible short peptides to connect two variable regions.

Accordingly, provided herein are bispecific antibodies that specifically bind to human TNFα and to human IL23p19, comprising: (1) a light chain (LC) comprising a first light chain variable domain (VL1), a second light chain variable domain (VL2), and a CL region; and (2) a heavy chain (HC) comprising a first heavy chain variable domain (VH1), a second heavy chain variable domain (VH2), and a CH region; wherein the VL1/VH1 pair and VL2/VH2 pair specifically bind to human TNFα and human IL23p19, respectively, or to human IL23p19 and human TNFα, respectively.

The amino acid sequences of the CL and the CH region of the bispecific antibodies disclosed herein can be derived from any appropriate source, e.g., a constant region of an antibody such as an IgG1, IgG2, IgG3, or IgG4. In some embodiments, the constant domains and constant regions of the bispecific antibodies provided herein are derived from human IgG. In some embodiments, the constant domains and constant regions of the bispecific antibodies provided herein are derived from human IgG1. In some embodiments, the constant domains and constant regions of the bispecific antibodies provided herein are derived from human IgG2. In some embodiments, the constant domains and constant regions of the bispecific antibodies provided herein are derived from human IgG3. In some embodiments, the constant domains and constant regions of the bispecific antibodies provided herein are derived from human IgG4. In some embodiments, the amino acid sequences of the CH1, the CL region, and the Fc region (hinge, CH2 and CH3) of the bispecific antibodies disclosed herein can comprise one or more amino acid substitutions that differ from the wild type immunoglobulin, e.g., one or more amino acid substitutions in a wild type IgG1 or IgG4. Such substitutions are known in the art (see, e.g., U.S. Pat. Nos. 7,704,497, 7,083,784, 6,821,505, 8,323,962, 6,737,056, and 7,416,727).

In some embodiments, the CH region can be selected from the group consisting of a human IgG1 CH region (SEQ ID NO: 58), a human IgG2 CH region (SEQ ID NO: 80), a human IgG3 CH region (SEQ ID NO: 81), and a human IgG4 CH region (SEQ ID NO: 82). In some embodiments, the CH region is human IgG1 CH region (SEQ ID NO: 58).

In some embodiments, the CL region can be kappa CL (Cκ; SEQ ID NO: 68). In some embodiments, the CL region can be lambda CL (Cλ, SEQ ID NO: 69).

In some embodiments of the bispecific antibodies provided herein, (1) the CL region is Cκ (SEQ ID NO: 68) or Cλ (SEQ ID NO: 69); or (2) the CH region is human IgG1 CH region (SEQ ID NO: 58); or both (1) and (2).

In some embodiments of the bispecific antibodies provided herein, the variable domains are linked together via a linker. In some embodiments, the VH1 is linked to VH2 via a linker. In some embodiments, the VL1 is linked to VL2 via a linker. In some embodiments, the linker has the amino acid sequence of (GGGGS)n, n=1, 2, 3, 4, or 5 (SEQ ID NO: 71). In some embodiments, the linker has the amino acid sequence of GGGGSGGGGSGGGGS (SEQ ID NO: 72). In some embodiments, the linker has the amino acid sequence of (EAAAK)n, n=1, 2, 3, 4, or 5 (SEQ ID NO: 73).

In some embodiments of the bispecific antibodies provided herein, the VL1/VH1 pair specifically binds to human TNFα and the VL2/VH2 pair specifically binds to human IL23p19. The VL1/VH1 pair can be any VL/VH pair that specifically binds to human TNFα. In some embodiments, the VL1 and VH1 are the VL and VH of adalimumab and have the amino acid sequences of SEQ ID NOs: 1 and 2, respectively. In some embodiments, the VL1 and VH1 are the VL and VH of golimumab and have the amino acid sequences of SEQ ID NOs: 5 and 6, respectively. The VL2/VH2 pair can be any VL/VH pair that specifically binds to human IL23p19. In some embodiments, the VL2 and VH2 are the VL and VH of guselkumab and have the amino acid sequences of SEQ ID NOs: 10 and 11, respectively. In some embodiments, the VL2 and VH2 are the VL and VH of tildrakizumab and have the amino acid sequences of SEQ ID NOs: 14 and 15, respectively.

In some embodiments of the bispecific antibodies provided herein, the VL1 and VH1 have the amino acid sequences of (1) SEQ ID NOs: 1 and 2, respectively; or (2) SEQ ID NOs: 5 and 6, respectively. In some embodiments, the VL1 and VH1 have the amino acid sequences of SEQ ID NOs: 1 and 2, respectively. In some embodiments, the VL1 and VH1 have the amino acid sequences of SEQ ID NOs: 5 and 6, respectively. In some embodiments of the bispecific antibodies provided herein, the VL2 and VH2 have the amino acid sequences of (1) SEQ ID NOs: 10 and 11, respectively; or (2) SEQ ID NOs: 14 and 15, respectively. In some embodiments, the VL2 and VH2 have the amino acid sequences of SEQ ID NOs: 10 and 11, respectively. In some embodiments, the VL2 and VH2 have the amino acid sequences of SEQ ID NOs: 14 and 15, respectively.

In some embodiments of the bispecific antibodies provided herein, the VL1, VH1, VL2 and VH2 have the amino acid sequences of (1) SEQ ID NOs: 1, 2, 10, and 11, respectively; (2) SEQ ID NOs: 1, 2, 14, and 15, respectively; (3) SEQ ID NOs: 5, 6, 10, and 11, respectively; or (4) SEQ ID NOs: 5, 6, 14, and 15, respectively. In some embodiments, the VL1, VH1, VL2 and VH2 have the amino acid sequences of SEQ ID NOs: 1, 2, 10, and 11, respectively. In some embodiments, the VL1, VH1, VL2 and VH2 have the amino acid sequences of SEQ ID NOs: 1, 2, 14, and 15, respectively. In some embodiments, the VL1, VH1, VL2 and VH2 have the amino acid sequences of SEQ ID NOs: 5, 6, 10, and 11, respectively. In some embodiments, the VL1, VH1, VL2 and VH2 have the amino acid sequences of SEQ ID NOs: 5, 6, 14, and 15, respectively.

In some embodiments of the bispecific antibodies provided herein, the VL1/VH1 pair specifically binds to human IL23p19 and the VL2/VH2 pair specifically binds to human TNFα. The VL1/VH1 pair can be any VL/VH pair that specifically binds to human IL23p19. In some embodiments, the VL1 and VH1 are the VL and VH of guselkumab and have the amino acid sequences of SEQ ID NOs: 10 and 11, respectively. In some embodiments, the VL1 and VH1 are the VL and VH of tildrakizumab and have the amino acid sequences of SEQ ID NOs: 14 and 15, respectively. The VL2/VH2 pair can be any VL/VH pair that specifically binds to human TNFα. In some embodiments, the VL2 and VH2 are the VL and VH of adalimumab and have the amino acid sequences of SEQ ID NOs: 1 and 2, respectively. In some embodiments, the VL2 and VH2 are the VL and VH of golimumab and have the amino acid sequences of SEQ ID NOs: 5 and 6, respectively.

In some embodiments of the bispecific antibodies provided herein, the VL1 and VHI have the amino acid sequences of (1) SEQ ID NOs: 10 and 11, respectively; or (2) SEQ ID NOs: 14 and 15, respectively. In some embodiments, the VL1 and VH1 have the amino acid sequences of SEQ ID NOs: 10 and 11, respectively. In some embodiments, the VL1 and VH1 have the amino acid sequences of SEQ ID NOs: 14 and 15, respectively. In some embodiments of the bispecific antibodies provided herein, the VL2 and VH-2 have the amino acid sequences of (1) SEQ ID NOs:1 and 2, respectively; or (2) SEQ ID NOs:5 and 6, respectively. In some embodiments, the VL2 and VH-2 have the amino acid sequences of SEQ ID NOs: 1 and 2, respectively. In some embodiments, the VL2 and VH42 have the amino acid sequences of SEQ ID NOs: 5 and 6, respectively.

In some embodiments of the bispecific antibodies provided herein, the VL1, VHI, VL2 and VH-2 have the amino acid sequences of (1) SEQ ID NOs: 10, 11, 1, and 2, respectively; (2) SEQ ID NOs: 10, 11, 5, and 6, respectively; (3) SEQ ID NOs: 14, 15, 1, and 2, respectively, or (4) SEQ ID NOs: 14, 15, 5, and 6, respectively. In some embodiments, the VL1, VH1, VL2 and VH-2 have the amino acid sequences of SEQ ID NOs: 10, 11, 1, and 2, respectively. In some embodiments, the VL1, VH1, VL2 and VH-2 have the amino acid sequences of SEQ ID NOs: 10, 11, 5, and 6, respectively. In some embodiments, the VL1, VHI, VL2 and VH-2 have the amino acid sequences of SEQ ID NOs: 14, 15, 1, and 2, respectively. In some embodiments, the VL1, VH1, VL2 and VH2 have the amino acid sequences of SEQ ID NOs: 14, 15, 5, and 6, respectively. A list of exemplary LC and HC of bispecific antibodies is provided below as Table 4C.

TABLE 4C
Exemplified Bispecific Antibodies to human TNFα and human IL23p19

| | | |
|---|---|---|
| C1 | LC | HC |
| | QSVLTQPPSVSGAPGQRVTISCT | EVQLVQSGAEVKKPGESLKISCKGSGYSFSNYWIG |
| | GSSSNIGSGYDVHWYQQLPGTAP | WVRQMPGKGLEWMGIIDPSNSYTRYSPSFQGQVTIS |
| | KLLIYGNSKRPSGVPDRFSGSKS | ADKSISTAYLQWSSLKASDTAMYYCARWYYKPFD |
| | GTSASLAITGLQSEDEADYYCAS | VWGQGTLVTVSSGGGGSGGGGSGGGGSEVQLVES |
| | WTDGLSLVVFGGGTKLTVLGGG | GGGLVQPGRSLRLSCAASGFTFDDYAMHWVRQAP |
| | GSGGGGSGGGGSDIQMTQSPSSL | GKGLEWVSAITWNSGHIDYADSVEGRFTISRDNAK |
| | SASVGDRVTITCRASQGIRNYLA | NSLYLQMNSLRAEDTAVYYCAKVSYLSTASSLDYW |
| | WYQQKPGKAPKLLIYAASTLQS | GQGTLVTVSSASTKGPSVFPLAPSSKSTSGGTAALG |
| | GVPSRFSGSGSGTDFTLTISSLQP | CLVKDYFPEPVTVSWNSGALTSGVHTFPAVLQSSGL |
| | EDVATYYCQRYNRAPYTFGQGT | YSLSSVVTVPSSSLGTQTYICNVNHKPSNTKVDKKV |
| | KVEIKRTVAAPSVFIFPPSDEQLK | EPKSCDKTHTCPPCPAPELLGGPSVFLFPPKPKDTLM |
| | SGTASVVCLLNNFYPREAKVQW | ISRTPEVTCVVVDVSHEDPEVKFNWYVDGVEVHNA |
| | KVDNALQSGNSQESVTEQDSKD | KTKPREEQYNSTYRVVSVLTVLHQDWLNGKEYKC |
| | STYSLSSTLTLSKADYEKHKVYA | KVSNKALPAPIEKTISKAKGQPREPQVYTLPPSRDEL |
| | CEVTHQGLSSPVTKSFNRGEC | TKNQVSLTCLVKGFYPSDIAVEWESNGQPENNYKT |
| | (SEQ ID NO: 38) | TPPVLDSDGSFFLYSKLTVDKSRWQQGNVFSCSVM |
| | | HEALHNHYTQKSLSLSPGK |
| | | (SEQ ID NO: 39) |
| C2 | LC | HC |
| | DIQMTQSPSSLSASVGDRVTITCR | QVQLVQSGAEVKKPGASVKVSCKASGYIFITYWMT |
| | TSENIYSYLAWYQQKPGKAPKLL | WVRQAPGQGLEWMGQIFPASGSADYNEKFEGRVT |
| | IYNAKTLAEGVPSRFSGSGSGTD | MTTDTSTSTAYMELRSLRSDDTAVYYCARGGGGFA |
| | FTLTISSLQPEDFATYYCQHHYGI | YWGQGTLVTVSSGGGGSGGGGSGGGGSEVQLVES |
| | PFTFGQGTKVEIKGGGGSGGGGS | GGGLVQPGRSLRLSCAASGFTFDDYAMHWVRQAP |
| | GGGGSDIQMTQSPSSLSASVGDR | GKGLEWVSAITWNSGHIDYADSVEGRFTISRDNAK |
| | VTITCRASQGIRNYLAWYQQKPG | NSLYLQMNSLRAEDTAVYYCAKVSYLSTASSLDYW |
| | KAPKLLIYAASTLQSGVPSRFSGS | GQGTLVTVSSASTKGPSVFPLAPSSKSTSGGTAALG |
| | GSGTDFTLTISSLQPEDVATYYC | CLVKDYFPEPVTVSWNSGALTSGVHTFPAVLQSSGL |
| | QRYNRAPYTFGQGTKVEIKRTV | YSLSSVVTVPSSSLGTQTYICNVNHKPSNTKVDKKV |
| | AAPSVFIFPPSDEQLKSGTASVVC | EPKSCDKTHTCPPCPAPELLGGPSVFLFPPKPKDTLM |
| | LLNNFYPREAKVQWKVDNALQS | ISRTPEVTCVVVDVSHEDPEVKFNWYVDGVEVHNA |
| | GNSQESVTEQDSKDSTYSLSSTL | KTKPREEQYNSTYRVVSVLTVLHQDWLNGKEYKC |
| | TLSKADYEKHKVYACEVTHQGL | KVSNKALPAPIEKTISKAKGQPREPQVYTLPPSRDEL |
| | SSPVTKSFNRGEC | TKNQVSLTCLVKGFYPSDIAVEWESNGQPENNYKT |
| | (SEQ ID NO: 40) | TPPVLDSDGSFFLYSKLTVDKSRWQQGNVFSCSVM |
| | | HEALHNHYTQKSLSLSPGK |
| | | (SEQ ID NO: 41) |
| C3 | LC | HC |
| | QSVLTQPPSVSGAPGQRVTISCT | EVQLVQSGAEVKKPGESLKISCKGSGYSFSNYWIG |
| | GSSSNIGSGYDVHWYQQLPGTAP | WVRQMPGKGLEWMGIIDPSNSYTRYSPSFQGQVTIS |
| | KLLIYGNSKRPSGVPDRFSGSKS | ADKSISTAYLQWSSLKASDTAMYYCARWYYKPFD |
| | GTSASLAITGLQSEDEADYYCAS | VWGQGTLVTVSSGGGGSGGGGSGGGGSQVQLVES |
| | WTDGLSLVVFGGGTKLTVLGGG | GGGVVQPGRSLRLSCAASGFIFSSYAMHWVRQAPG |
| | GSGGGGSGGGGSEIVLTQSPATL | NGLEWVAFMSYDGSNKKYADSVKGRFTISRDNSKN |
| | SLSPGERATLSCRASQSVYSYLA | TLYLQMNSLRAEDTAVYYCARDRGIAAGGNYYYY |
| | WYQQKPGQAPRLLIYDASNRAT | GMDVWGQGTTVTVSSASTKGPSVFPLAPSSKSTSG |
| | GIPARFSGSGSGTDFTLTISSLEPE | GTAALGCLVKDYFPEPVTVSWNSGALTSGVHTFPA |
| | DFAVYYCQQRSNWPPFTFGPGT | VLQSSGLYSLSSVVTVPSSSLGTQTYICNVNHKPSNT |
| | KVDIKRTVAAPSVFIFPPSDEQLK | KVDKKVEPKSCDKTHTCPPCPAPELLGGPSVFLFPP |
| | SGTASVVCLLNNFYPREAKVQW | KPKDTLMISRTPEVTCVVVDVSHEDPEVKFNWYVD |
| | KVDNALQSGNSQESVTEQDSKD | GVEVHNAKTKPREEQYNSTYRVVSVLTVLHQDWL |
| | STYSLSSTLTLSKADYEKHKVYA | NGKEYKCKVSNKALPAPIEKTISKAKGQPREPQVYT |
| | CEVTHQGLSSPVTKSFNRGEC | LPPSRDELTKNQVSLTCLVKGFYPSDIAVEWESNGQ |
| | (SEQ ID NO: 42) | PENNYKTTPPVLDSDGSFFLYSKLTVDKSRWQQGN |
| | | VFSCSVMHEALHNHYTQKSLSLSPGK |
| | | (SEQ ID NO: 43) |
| C4 | LC | HC |
| | DIQMTQSPSSLSASVGDRVTITCR | QVQLVQSGAEVKKPGASVKVSCKASGYIFITYWMT |
| | TSENIYSYLAWYQQKPGKAPKLL | WVRQAPGQGLEWMGQIFPASGSADYNEKFEGRVT |
| | IYNAKTLAEGVPSRFSGSGSGTD | MTTDTSTSTAYMELRSLRSDDTAVYYCARGGGGFA |
| | FTLTISSLQPEDFATYYCQHHYGI | YWGQGTLVTVSSGGGGSGGGGSGGGGSQVQLVES |
| | PFTFGQGTKVEIKGGGGSGGGGS | GGGVVQPGRSLRLSCAASGFIFSSYAMHWVRQAPG |
| | GGGGSEIVLTQSPATLSLSPGERA | NGLEWVAFMSYDGSNKKYADSVKGRFTISRDNSKN |
| | TLSCRASQSVYSYLAWYQQKPG | TLYLQMNSLRAEDTAVYYCARDRGIAAGGNYYYY |
| | QAPRLLIYDASNRATGIPARFSGS | GMDVWGQGTTVTVSSASTKGPSVFPLAPSSKSTSG |
| | GSGTDFTLTISSLEPEDFAVYYCQ | GTAALGCLVKDYFPEPVTVSWNSGALTSGVHTFPA |
| | QRSNWPPFTFGPGTKVDIKRTVA | VLQSSGLYSLSSVVTVPSSSLGTQTYICNVNHKPSNT |
| | APSVFIFPPSDEQLKSGTASVVCL | KVDKKVEPKSCDKTHTCPPCPAPELLGGPSVFLFPP |
| | LNNFYPREAKVQWKVDNALQSG | KPKDTLMISRTPEVTCVVVDVSHEDPEVKFNWYVD |
| | NSQESVTEQDSKDSTYSLSSTLTL | GVEVHNAKTKPREEQYNSTYRVVSVLTVLHQDWL |
| | SKADYEKHKVYACEVTHQGLSS | NGKEYKCKVSNKALPAPIEKTISKAKGQPREPQVYT |
| | PVTKSFNRGEC | LPPSRDELTKNQVSLTCLVKGFYPSDIAVEWESNGQ |
| | (SEQ ID NO: 44) | PENNYKTTPPVLDSDGSFFLYSKLTVDKSRWQQGN |
| | | VFSCSVMHEALHNHYTQKSLSLSPGK |
| | | (SEQ ID NO: 45) |
| C5 | LC | HC |
| | DIQMTQSPSSLSASVGDRVTITCR | EVQLVESGGGLVQPGRSLRLSCAASGFTFDDYAMH |
| | ASQGIRNYLAWYQQKPGKAPKL | WVRQAPGKGLEWVSAITWNSGHIDYADSVEGRFTI |
| | LIYAASTLQSGVPSRFSGSGSGTD | SRDNAKNSLYLQMNSLRAEDTAVYYCAKVSYLSTA |
| | FTLTISSLQPEDVATYYCQRYNR | SSLDYWGQGTLVTVSSGGGGSGGGGSGGGGSEVQL |
| | APYTFGQGTKVEIKGGGGSGGG | VQSGAEVKKPGESLKISCKGSGYSFSNYWIGWVRQ |
| | GSGGGGSQSVLTQPPSVSGAPGQ | MPGKGLEWMGIIDPSNSYTRYSPSFQGQVTISADKSI |
| | RVTISCTGSSSNIGSGYDVHWYQ | STAYLQWSSLKASDTAMYYCARWYYKPFDVWGQ |
| | QLPGTAPKLLIYGNSKRPSGVPD | GTLVTVSSASTKGPSVFPLAPSSKSTSGGTAALGCLV |
| | RFSGSKSGTSASLAITGLQSEDEA | KDYFPEPVTVSWNSGALTSGVHTFPAVLQSSGLYSL |
| | DYYCASWTDGLSLVVFGGGTKL | SSVVTVPSSSLGTQTYICNVNHKPSNTKVDKKVEPK |
| | TVLGQPKAAPSVTLFPPSSEELQA | SCDKTHTCPPCPAPELLGGPSVFLFPPKPKDTLMISR |
| | NKATLVCLISDFYPGAVTVAWK | TPEVTCVVVDVSHEDPEVKFNWYVDGVEVHNAKT |
| | ADSSPVKAGVETTTPSKQSNNKY | KPREEQYNSTYRVVSVLTVLHQDWLNGKEYKCKV |
| | AASSYLSLTPEQWKSHRSYSCQV | SNKALPAPIEKTISKAKGQPREPQVYTLPPSRDELTK |
| | THEGSTVEKTVAPTECS | NQVSLTCLVKGFYPSDIAVEWESNGQPENNYKTTPP |
| | (SEQ ID NO: 46) | VLDSDGSFFLYSKLTVDKSRWQQGNVFSCSVMHEA |
| | | LHNHYTQKSLSLSPGK |
| | | (SEQ ID NO: 47) |
| C6 | LC | HC |
| | EIVLTQSPATLSLSPGERATLSCR | QVQLVESGGGVVQPGRSLRLSCAASGFIFSSYAMH |
| | ASQSVYSYLAWYQQKPGQAPRL | WVRQAPGNGLEWVAFMSYDGSNKKYADSVKGRF |
| | LIYDASNRATGIPARFSGSGSGTD | TISRDNSKNTLYLQMNSLRAEDTAVYYCARDRGIA |
| | FTLTISSLEPEDFAVYYCQQRSN | AGGNYYYYGMDVWGQGTTVTVSSGGGGSGGGGS |
| | WPPFTFGPGTKVDIKGGGGSGGG | GGGGSEVQLVQSGAEVKKPGESLKISCKGSGYSFSN |
| | GSGGGGSQSVLTQPPSVSGAPGQ | YWIGWVRQMPGKGLEWMGIIDPSNSYTRYSPSFQG |
| | RVTISCTGSSSNIGSGYDVHWYQ | QVTISADKSISTAYLQWSSLKASDTAMYYCARWYY |
| | QLPGTAPKLLIYGNSKRPSGVPD | KPFDVWGQGTLVTVSSASTKGPSVFPLAPSSKSTSG |
| | RFSGSKSGTSASLAITGLQSEDEA | GTAALGCLVKDYFPEPVTVSWNSGALTSGVHTFPA |
| | DYYCASWTDGLSLVVFGGGTKL | VLQSSGLYSLSSVVTVPSSSLGTQTYICNVNHKPSNT |
| | TVLGQPKAAPSVTLFPPSSEELQA | KVDKKVEPKSCDKTHTCPPCPAPELLGGPSVFLFPP |
| | NKATLVCLISDFYPGAVTVAWK | KPKDTLMISRTPEVTCVVVDVSHEDPEVKFNWYVD |
| | ADSSPVKAGVETTTPSKQSNNKY | GVEVHNAKTKPREEQYNSTYRVVSVLTVLHQDWL |
| | AASSYLSLTPEQWKSHRSYSCQV | NGKEYKCKVSNKALPAPIEKTISKAKGQPREPQVYT |
| | THEGSTVEKTVAPTECS | LPPSRDELTKNQVSLTCLVKGFYPSDIAVEWESNGQ |
| | (SEQ ID NO: 48) | PENNYKTTPPVLDSDGSFFLYSKLTVDKSRWQQGN |
| | | VFSCSVMHEALHNHYTQKSLSLSPGK |
| | | (SEQ ID NO: 49) |
| C7 | LC | HC |
| | DIQMTQSPSSLSASVGDRVTITCR | EVQLVESGGGLVQPGRSLRLSCAASGFTFDDYAMH |
| | ASQGIRNYLAWYQQKPGKAPKL | WVRQAPGKGLEWVSAITWNSGHIDYADSVEGRFTI |
| | LIYAASTLQSGVPSRFSGSGSGTD | SRDNAKNSLYLQMNSLRAEDTAVYYCAKVSYLSTA |
| | FTLTISSLQPEDVATYYCQRYNR | SSLDYWGQGTLVTVSSGGGGSGGGGSGGGGSQVQ |
| | APYTFGQGTKVEIKGGGGSGGG | LVQSGAEVKKPGASVKVSCKASGYIFITYWMTWVR |
| | GSGGGGSDIQMTQSPSSLSASVG | QAPGQGLEWMGQIFPASGSADYNEKFEGRVTMTTD |
| | DRVTITCRTSENIYSYLAWYQQK | TSTSTAYMELRSLRSDDTAVYYCARGGGGFAYWG |
| | PGKAPKLLIYNAKTLAEGVPSRF | QGTLVTVSSASTKGPSVFPLAPSSKSTSGGTAALGCL |
| | SGSGSGTDFTLTISSLQPEDFATY | VKDYFPEPVTVSWNSGALTSGVHTFPAVLQSSGLYS |
| | YCQHHYGIPFTFGQGTKVEIKRT | LSSVVTVPSSSLGTQTYICNVNHKPSNTKVDKKVEP |
| | VAAPSVFIFPPSDEQLKSGTASVV | KSCDKTHTCPPCPAPELLGGPSVFLFPPKPKDTLMIS |
| | CLLNNFYPREAKVQWKVDNALQ | RTPEVTCVVVDVSHEDPEVKFNWYVDGVEVHNAK |
| | SGNSQESVTEQDSKDSTYSLSSTL | TKPREEQYNSTYRVVSVLTVLHQDWLNGKEYKCK |
| | TLSKADYEKHKVYACEVTHQGL | VSNKALPAPIEKTISKAKGQPREPQVYTLPPSRDELT |
| | SSPVTKSFNRGEC | KNQVSLTCLVKGFYPSDIAVEWESNGQPENNYKTT |
| | (SEQ ID NO: 50) | PPVLDSDGSFFLYSKLTVDKSRWQQGNVFSCSVMH |
| | | EALHNHYTQKSLSLSPGK |
| | | (SEQ ID NO: 51) |
| C8 | LC | HC |
| | EIVLTQSPATLSLSPGERATLSCR | QVQLVESGGGVVQPGRSLRLSCAASGFIFSSYAMH |
| | ASQSVYSYLAWYQQKPGQAPRL | WVRQAPGNGLEWVAFMSYDGSNKKYADSVKGRF |
| | LIYDASNRATGIPARFSGSGSGTD | TISRDNSKNTLYLQMNSLRAEDTAVYYCARDRGIA |
| | FTLTISSLEPEDFAVYYCQQRSN | AGGNYYYYGMDVWGQGTTVTVSSGGGGSGGGGS |
| | WPPFTFGPGTKVDIKGGGGSGGG | GGGGSQVQLVQSGAEVKKPGASVKVSCKASGYIFIT |
| | GSGGGGSDIQMTQSPSSLSASVG | YWMTWVRQAPGQGLEWMGQIFPASGSADYNEKFE |
| | DRVTITCRTSENIYSYLAWYQQK | GRVTMTTDTSTSTAYMELRSLRSDDTAVYYCARGG |
| | PGKAPKLLIYNAKTLAEGVPSRF | GGFAYWGQGTLVTVSSASTKGPSVFPLAPSSKSTSG |
| | SGSGSGTDFTLTISSLQPEDFATY | GTAALGCLVKDYFPEPVTVSWNSGALTSGVHTFPA |
| | YCQHHYGIPFTFGQGTKVEIKRT | VLQSSGLYSLSSVVTVPSSSLGTQTYICNVNHKPSNT |
| | VAAPSVFIFPPSDEQLKSGTASVV | KVDKKVEPKSCDKTHTCPPCPAPELLGGPSVFLFPP |
| | CLLNNFYPREAKVQWKVDNALQ | KPKDTLMISRTPEVTCVVVDVSHEDPEVKFNWYVD |
| | SGNSQESVTEQDSKDSTYSLSSTL | GVEVHNAKTKPREEQYNSTYRVVSVLTVLHQDWL |
| | TLSKADYEKHKVYACEVTHQGL | NGKEYKCKVSNKALPAPIEKTISKAKGQPREPQVYT |
| | SSPVTKSFNRGEC | LPPSRDELTKNQVSLTCLVKGFYPSDIAVEWESNGQ |
| | (SEQ ID NO: 52) | PENNYKTTPPVLDSDGSFFLYSKLTVDKSRWQQGN |
| | | VFSCSVMHEALHNHYTQKSLSLSPGK |
| | | (SEQ ID NO: 53) |

In some embodiments, provided herein are bispecific antibody that specifically bind to human TNFα and to human IL23p19 having LC and HC, wherein LC and HC each has at least 90%, at least 95%, at least 98%, at least 99%, or 100% sequence identity to the amino acid sequences of (1) SEQ ID NOs: 38 and 39, respectively; (2) SEQ ID NOs: 40 and 41, respectively; (3) SEQ ID NOs: 42 and 43, respectively; (4) SEQ ID NOs: 44 and 45, respectively; (5) SEQ ID NOs: 46 and 47, respectively; (6) SEQ ID NOs: 48 and 49 respectively; (7) SEQ ID NOs: 50 and 51, respectively; or (8) SEQ ID NOs: 52 and 53, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs: 38 and 39, respectively. In some embodiments, the LC has at least 90%, at least 95%, at least 98%, at least 99%, or 100% sequence identity to SEQ ID NO: 38. In some embodiments, the LC has the amino acid sequence of SEQ ID NO: 38. In some embodiments, the HC has at least 90%, at least 95%, at least 98%, at least 99%, or 100% sequence identity to SEQ ID NO: 39. In some embodiments, the HC has the amino acid sequence of SEQ ID NO: 39. In some embodiments, the LC and HC each has at least 90% sequence identity to the amino acid sequences of SEQ ID NOs: 38 and 39, respectively. In some embodiments, the LC and HC each has at least 95% sequence identity to the amino acid sequences of SEQ ID NOs: 38 and 39, respectively. In some embodiments, the LC and HC each has at least 98% sequence identity to the amino acid sequences of SEQ ID NOs: 38 and 39, respectively. In some embodiments, the LC and HC each has at least 99% sequence identity to the amino acid sequences of SEQ ID NOs: 38 and 39, respectively. In some embodiments, provided herein is the bispecific antibody designated as C1, of which the LC and HC have the amino acid sequences of SEQ ID NOs: 38 and 39, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:40 and 41, respectively. In some embodiments, the LC has at least 90%, at least 95%, at least 98%, at least 99%, or 100% sequence identity to SEQ ID NO: 40. In some embodiments, the LC has the amino acid sequence of SEQ ID NO: 40. In some embodiments, the HC has at least 90%, at least 95%, at least 98%, at least 99%, or 100% sequence identity to SEQ ID NO: 41. In some embodiments, the HC has the amino acid sequence of SEQ ID NO: 41. In some embodiments, the LC and HC each has at least 90% sequence identity to the amino acid sequences of SEQ ID NOs: 40 and 41, respectively. In some embodiments, the LC and HC each has at least 95% sequence identity to the amino acid sequences of SEQ ID NOs: 40 and 41, respectively. In some embodiments, the LC and HC each has at least 98% sequence identity to the amino acid sequences of SEQ ID NOs: 40 and 41, respectively. In some embodiments, the LC and HC each has at least 99% sequence identity to the amino acid sequences of SEQ ID NOs: 40 and 41, respectively. In some embodiments, provided herein is the bispecific antibody designated as C2, of which the LC and HC have the amino acid sequences of SEQ ID NOs: 40 and 41, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:42 and 43, respectively. In some embodiments, the LC has at least 90%, at least 95%, at least 98%, at least 99%, or 100% sequence identity to SEQ ID NO: 42. In some embodiments, the LC has the amino acid sequence of SEQ ID NO: 42. In some embodiments, the HC has at least 90%, at least 95%, at least 98%, at least 99%, or 100% sequence identity to SEQ ID NO: 43. In some embodiments, the HC has the amino acid sequence of SEQ ID NO: 43. In some embodiments, the LC and HC each has at least 90% sequence identity to the amino acid sequences of SEQ ID NOs: 42 and 43, respectively. In some embodiments, the LC and HC each has at least 95% sequence identity to the amino acid sequences of SEQ ID NOs: 42 and 43, respectively. In some embodiments, the LC and HC each has at least 98% sequence identity to the amino acid sequences of SEQ ID NOs: 42 and 43, respectively. In some embodiments, the LC and HC each has at least 99% sequence identity to the amino acid sequences of SEQ ID NOs: 42 and 43, respectively. In some embodiments, provided herein is the bispecific antibody designated as C3, of which the LC and HC have the amino acid sequences of SEQ ID NOs: 42 and 43, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:44 and 45, respectively. In some embodiments, the LC has at least 90%, at least 95% at least 98%, at least 99%, or 100% sequence identity to SEQ ID NO: 44. In some embodiments, the LC has the amino acid sequence of SEQ ID NO: 44. In some embodiments, the HC has at least 90%, at least 95%, at least 98%, at least 99%, or 100% sequence identity to SEQ ID NO: 45. In some embodiments, the HC has the amino acid sequence of SEQ ID NO: 45. In some embodiments, the LC and HC each has at least 90% sequence identity to the amino acid sequences of SEQ ID NOs: 44 and 45, respectively. In some embodiments, the LC and HC each has at least 95% sequence identity to the amino acid sequences of SEQ ID NOs: 44 and 45, respectively. In some embodiments, the LC and HC each has at least 98% sequence identity to the amino acid sequences of SEQ ID NOs: 44 and 45, respectively. In some embodiments, the LC and HC each has at least 99% sequence identity to the amino acid sequences of SEQ ID NOs: 44 and 45, respectively. In some embodiments, provided herein is the bispecific antibody designated as C4, of which the LC and HC have the amino acid sequences of SEQ ID NOs: 44 and 45, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs: 46 and 47, respectively. In some embodiments, the LC has at least 90%, at least 95%, at least 98%, at least 99%, or 100% sequence identity to SEQ ID NO: 46. In some embodiments, the LC has the amino acid sequence of SEQ ID NO: 46. In some embodiments, the HC has at least 90%, at least 95%, at least 98%, at least 99%, or 100% sequence identity to SEQ ID NO: 47. In some embodiments, the HC has the amino acid sequence of SEQ ID NO: 47. In some embodiments, the LC and HC each has at least 90% sequence identity to the amino acid sequences of SEQ ID NOs: 46 and 47, respectively. In some embodiments, the LC and HC each has at least 95% sequence identity to the amino acid sequences of SEQ ID NOs: 46 and 47, respectively. In some embodiments, the LC and HC each has at least 98% sequence identity to the amino acid sequences of SEQ ID NOs: 46 and 47, respectively. In some embodiments, the LC and HC each has at least 99% sequence identity to the amino acid sequences of SEQ ID NOs: 46 and 47, respectively. In some embodiments, provided herein is the bispecific antibody designated as C5, of which the LC and HC have the amino acid sequences of SEQ ID NOs: 46 and 47, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:48 and 49, respectively. In some embodiments, the LC has at least 90%, at least 95% at least 98%, at least 99%, or 100% sequence identity to SEQ ID NO: 48. In some embodiments, the LC has the amino acid sequence of SEQ ID NO: 48. In some embodiments, the HC has at least 90%, at least 95%, at least 98%, at least 99%, or 100% sequence identity to SEQ ID NO: 49. In some embodiments, the HC has the amino acid sequence of SEQ ID NO: 49. In some embodiments, the LC and HC each has at least 90% sequence identity to the amino acid sequences of SEQ ID NOs: 48 and 49, respectively. In some embodiments, the LC and HC each has at least 95% sequence identity to the amino acid sequences of SEQ ID NOs: 48 and 49, respectively. In some embodiments, the LC and HC each has at least 98% sequence identity to the amino acid sequences of SEQ ID NOs: 48 and 49, respectively. In some embodiments, the LC and HC each has at least 99% sequence identity to the amino acid sequences of SEQ ID NOs: 48 and 49, respectively. In some embodiments, provided herein is the bispecific antibody designated as C6, of which the LC and HC have the amino acid sequences of SEQ ID NOs: 48 and 49, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:50 and 51, respectively. In some embodiments, the LC has at least 90%, at least 95%, at least 98%, at least 99%, or 100% sequence identity to SEQ ID NO: 50. In some embodiments, the LC has the amino acid sequence of SEQ ID NO: 50. In some embodiments, the HC has at least 90%, at least 95%, at least 98%, at least 99%, or 100% sequence identity to SEQ ID NO: 51. In some embodiments, the HC has the amino acid sequence of SEQ ID NO: 51. In some embodiments, the LC and HC each has at least 90% sequence identity to the amino acid sequences of SEQ ID NOs: 50 and 51, respectively. In some embodiments, the LC and HC each has at least 95% sequence identity to the amino acid sequences of SEQ ID NOs: 50 and 51, respectively. In some embodiments, the LC and HC each has at least 98% sequence identity to the amino acid sequences of SEQ ID NOs: 50 and 51, respectively. In some embodiments, the LC and HC each has at least 99% sequence identity to the amino acid sequences of SEQ ID NOs: 50 and 51, respectively. In some embodiments, provided herein is the bispecific antibody designated as C7, of which the LC and HC have the amino acid sequences of SEQ ID NOs: 50 and 51, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:52 and 53, respectively. In some embodiments, the LC has at least 90%, at least 95%, at least 98%, at least 99%, or 100% sequence identity to SEQ ID NO: 52. In some embodiments, the LC has the amino acid sequence of SEQ ID NO: 52. In some embodiments, the HC has at least 90%, at least 95%, at least 98%, at least 99%, or 100% sequence identity to SEQ ID NO: 53. In some embodiments, the HC has the amino acid sequence of SEQ ID NO: 53. In some embodiments, the LC and HC each has at least 90% sequence identity to the amino acid sequences of SEQ ID NOs: 52 and 53, respectively. In some embodiments, the LC and HC each has at least 95% sequence identity to the amino acid sequences of SEQ ID NOs: 52 and 53, respectively. In some embodiments, the LC and HC each has at least 98% sequence identity to the amino acid sequences of SEQ ID NOs: 52 and 53, respectively. In some embodiments, the LC and HC each has at least 99% sequence identity to the amino acid sequences of SEQ ID NOs: 52 and 53, respectively. In some embodiments, provided herein is the bispecific antibody designated as C8, of which the LC and HC have the amino acid sequences of SEQ ID NOs: 52 and 53, respectively.

### 2.5 SMAB

In some embodiments, the bispecific antibodies provided herein that specifically bind to human TNFα and to human IL23p19 have the “Single-Domain Antibody fused to Monoclonal Antibody” or “SMAB” structure depicted in FIG. 1D. The single-domain antibody can be a single heavy chain variable domain antibody (“VHH”). The VHH can be linked to the heavy chain or light chain of the IgG. In some embodiments, the VHH is linked to the heavy chain of the IgG. In some embodiments, the VHH is linked to the N-terminus of the IgG heavy chain. In some embodiments, the VHH is linked to the C-terminus of the IgG heavy chain. In some embodiments, the VHH is linked to the light chain of the IgG. In some embodiments, the VHH is linked to the N-terminus of the IgG light chain. In some embodiments, the VHH is linked to the C-terminus of the IgG light chain.

Accordingly, provided herein are bispecific antibodies that specifically bind to human TNFα and human IL23p19, comprising: (1) a light chain (LC) comprising a light chain variable domain (VL) and a CL region; and (2) a heavy chain (HC) comprising a heavy chain variable domain (VH) and a CH region; wherein (1) the VL/VH pair specifically binds to human IL23p19; and (2) the HC further comprises a VHH that specifically binds to human TNFα. In some embodiments, the VHH is linked to the N-terminus of the HC. In some embodiments, the VHH is linked to the C-terminus of the HC.

Provided herein are also bispecific antibodies that specifically bind to human TNFα and human IL23p19, comprising: (1) a light chain (LC) comprising a light chain variable domain (VL) and a CL region; and (2) a heavy chain (HC) comprising a heavy chain variable domain (VH) and a CH region; wherein (1) the VL/VH pair specifically binds to human TNFα; and (2) the HC further comprises a VHH that specifically binds to human IL23p19. In some embodiments, the VHH is linked to the N-terminus of the HC. In some embodiments, the VHH is linked to the C-terminus of the HC.

Provided herein are also bispecific antibodies that specifically bind to human TNFα and human IL23p19, comprising: (1) a light chain (LC) comprising a light chain variable domain (VL) and a CL region; and (2) a heavy chain (HC) comprising a heavy chain variable domain (VH) and a CH region; wherein (1) the VL/VH pair specifically binds to human IL23p19; and (2) the LC further comprises a VHH that specifically binds to human TNFα. In some embodiments, the VHH is linked to the N-terminus of the HC. In some embodiments, the VHH is linked to the C-terminus of the LC.

Provided herein are also bispecific antibodies that specifically bind to human TNFα and human IL23p19, comprising: (1) a light chain (LC) comprising a light chain variable domain (VL) and a CL region; and (2) a heavy chain (HC) comprising a heavy chain variable domain (VH) and a CH region; wherein (1) the VL/VH pair specifically binds to human TNFα; and (2) the LC further comprises a VHH that specifically binds to human IL23p19. In some embodiments, the VHH is linked to the N-terminus of the LC. In some embodiments, the VHH is linked to the C-terminus of the LC.

The amino acid sequences of the CL and the CH region of the bispecific antibodies disclosed herein can be derived from any appropriate source, e.g., a constant region of an antibody such as an IgG1, IgG2, IgG3, or IgG4. In some embodiments, the constant domains and constant regions of the bispecific antibodies provided herein are derived from human IgG. In some embodiments, the constant domains and constant regions of the bispecific antibodies provided herein are derived from human IgG1. In some embodiments, the constant domains and constant regions of the bispecific antibodies provided herein are derived from human IgG2. In some embodiments, the constant domains and constant regions of the bispecific antibodies provided herein are derived from human IgG3. In some embodiments, the constant domains and constant regions of the bispecific antibodies provided herein are derived from human IgG4. In some embodiments, the amino acid sequences of the CH1, the CL region, and the Fc region (the hinge, CH2 and CH3) of the bispecific antibodies disclosed herein can comprise one or more amino acid substitutions that differ from the wild type immunoglobulin, e.g., one or more amino acid substitutions in a wild type IgG1 or IgG4. Such substitutions are known in the art (see, e.g., U.S. Pat. Nos. 7,704,497, 7,083,784, 6,821,505, 8,323,962, 6,737,056, and 7,416,727).

In some embodiments, the CH region can be selected from the group consisting of a human IgG1 CH region (SEQ ID NO: 58), a human IgG2 CH region (SEQ ID NO: 80), a human IgG3 CH region (SEQ ID NO: 81), and a human IgG4 CH region (SEQ ID NO: 82). In some embodiments, the CH region is human IgG1 CH region (SEQ ID NO: 58).

In some embodiments, the CL region can be kappa CL (Cκ; SEQ ID NO: 68). In some embodiments, the CL region can be lambda CL (Cλ, SEQ ID NO: 69).

In some embodiments of the bispecific antibodies provided herein, (1) the CL region is Cκ (SEQ ID NO: 68) or Cλ (SEQ ID NO: 69); or (2) the CH region is human IgG1 CH region (SEQ ID NO: 58); or both (1) and (2).

In some embodiments of the bispecific antibodies provided herein, the VHH is linked to the IgG heavy chain via a linker. In some embodiments, the C-terminus of the VHH is linked to N-terminus of the VH via a linker. In some embodiments, the N-terminus VHH is linked to the C-terminus of the CH region via a linker. In some embodiments of the bispecific antibodies provided herein, the VHH is linked to the IgG light chain via a linker. In some embodiments, the C-terminus of the VHH is linked to N-terminus of the VL via a linker. In some embodiments, the N-terminus VHH is linked to the C-terminus of the CL region via a linker. In some embodiments, the linker has the amino acid sequence of (GGGGS)n, n=1, 2, 3, 4, or 5 (SEQ ID NO:71). In some embodiments, the linker has the amino acid sequence of GGGGSGGGGSGGGGS (SEQ ID NO: 72). In some embodiments, the linker has the amino acid sequence of (EAAAK)n, n=1, 2, 3, 4, or 5 (SEQ ID NO: 73).

In some embodiments, the bispecific antibodies provided herein comprise (1) a light chain (LC) comprising a light chain variable domain (VL) and a CL region; and (2) a heavy chain (HC) comprising a heavy chain variable domain (VH) and a CH region; wherein (1) the VL/VH pair specifically binds to human IL23p19; and (2) the HC further comprises a VHH that specifically binds to human TNFα. In some embodiments, the VHH is linked to the N-terminus of the HC. In some embodiments, the VHH is linked to the C-terminus of the HC. The VL/VH pair can be any VL/VH pair that specifically binds to human IL23p19. In some embodiments, the VL and VH are the VL and VH of guselkumab and have the amino acid sequences of SEQ ID NOs: 10 and 11, respectively. In some embodiments, the VL and VHI are the VL and VH of tildrakizumab and have the amino acid sequences of SEQ ID NOs: 14 and 15, respectively. The VHH can be any VHH that specifically binds to human TNFα. In some embodiments, the VHH can be ozoralizumab (SEQ ID NO: 9).

In some embodiments of the bispecific antibodies provided herein, the VL and VH have the amino acid sequences of SEQ ID NOs: 10 and 11, respectively, and the VHH has the amino acid sequence of SEQ ID NO: 9. In some embodiments, the VL and VH have the amino acid sequences of SEQ ID NOs: 14 and 15, respectively, and the VHH has the amino acid sequence of SEQ ID NO: 9. A list of exemplary LC and HC of bispecific antibodies is provided below as Table 4D.

TABLE 4D
Exemplified Bispecific Antibodies to human TNFα and human IL23p19

| | | |
|---|---|---|
| D1 | LC | HC |
| | QSVLTQPPSVSGAPGQRVTI | EVQLVESGGGLVQPGGSLRLSCAASGFTFSDYWMYWVRQAP |
| | SCTGSSSNIGSGYDVHWYQ | GKGLEWVSEINTNGLITKYPDSVKGRFTISRDNAKNTLYLQM |
| | QLPGTAPKLLIYGNSKRPSG | NSLRPEDTAVYYCARSPSGFNRGQGTLVTVSSGGGGSGGGGS |
| | VPDRFSGSKSGTSASLAITG | GGGGSEVQLVQSGAEVKKPGESLKISCKGSGYSFSNYWIGWV |
| | LQSEDEADYYCASWTDGLS | RQMPGKGLEWMGIIDPSNSYTRYSPSFQGQVTISADKSISTAY |
| | LVVFGGGTKLTVLGQPKAA | LQWSSLKASDTAMYYCARWYYKPFDVWGQGTLVTVSSAST |
| | PSVTLFPPSSEELQANKATL | KGPSVFPLAPSSKSTSGGTAALGCLVKDYFPEPVTVSWNSGA |
| | VCLISDFYPGAVTVAWKAD | LTSGVHTFPAVLQSSGLYSLSSVVTVPSSSLGTQTYICNVNHK |
| | SSPVKAGVETTTPSKQSNNK | PSNTKVDKKVEPKSCDKTHTCPPCPAPELLGGPSVFLFPPKPK |
| | YAASSYLSLTPEQWKSHRS | DTLMISRTPEVTCVVVDVSHEDPEVKFNWYVDGVEVHNAKT |
| | YSCQVTHEGSTVEKTVAPT | KPREEQYNSTYRVVSVLTVLHQDWLNGKEYKCKVSNKALPA |
| | ECS | PIEKTISKAKGQPREPQVYTLPPSRDELTKNQVSLTCLVKGFYP |
| | (SEQ ID NO: 12) | SDIAVEWESNGQPENNYKTTPPVLDSDGSFFLYSKLTVDKSR |
| | | WQQGNVFSCSVMHEALHNHYTQKSLSLSPGK |
| | | (SEQ ID NO: 54) |
| D2 | LC | HC |
| | QSVLTQPPSVSGAPGQRVTI | EVQLVQSGAEVKKPGESLKISCKGSGYSFSNYWIGWVRQMP |
| | SCTGSSSNIGSGYDVHWYQ | GKGLEWMGIIDPSNSYTRYSPSFQGQVTISADKSISTAYLQWS |
| | QLPGTAPKLLIYGNSKRPSG | SLKASDTAMYYCARWYYKPFDVWGQGTLVTVSSASTKGPSV |
| | VPDRFSGSKSGTSASLAITG | FPLAPSSKSTSGGTAALGCLVKDYFPEPVTVSWNSGALTSGV |
| | LQSEDEADYYCASWTDGLS | HTFPAVLQSSGLYSLSSVVTVPSSSLGTQTYICNVNHKPSNTK |
| | LVVFGGGTKLTVLGQPKAA | VDKKVEPKSCDKTHTCPPCPAPELLGGPSVFLFPPKPKDTLMI |
| | PSVTLFPPSSEELQANKATL | SRTPEVTCVVVDVSHEDPEVKFNWYVDGVEVHNAKTKPREE |
| | VCLISDFYPGAVTVAWKAD | QYNSTYRVVSVLTVLHQDWLNGKEYKCKVSNKALPAPIEKTI |
| | SSPVKAGVETTTPSKQSNNK | SKAKGQPREPQVYTLPPSRDELTKNQVSLTCLVKGFYPSDIAV |
| | YAASSYLSLTPEQWKSHRS | EWESNGQPENNYKTTPPVLDSDGSFFLYSKLTVDKSRWQQG |
| | YSCQVTHEGSTVEKTVAPT | NVFSCSVMHEALHNHYTQKSLSLSPGKGGGGSGGGGSGGGG |
| | ECS | SEVQLVESGGGLVQPGGSLRLSCAASGFTFSDYWMYWVRQA |
| | (SEQ ID NO: 12) | PGKGLEWVSEINTNGLITKYPDSVKGRFTISRDNAKNTLYLQ |
| | | MNSLRPEDTAVYYCARSPSGFNRGQGTLVTVSS |
| | | (SEQ ID NO: 55) |
| D3 | LC | HC |
| | DIQMTQSPSSLSASVGDRVT | EVQLVESGGGLVQPGGSLRLSCAASGFTFSDYWMYWVRQAP |
| | ITCRTSENIYSYLAWYQQKP | GKGLEWVSEINTNGLITKYPDSVKGRFTISRDNAKNTLYLQM |
| | GKAPKLLIYNAKTLAEGVPS | NSLRPEDTAVYYCARSPSGFNRGQGTLVTVSSGGGGSGGGGS |
| | RFSGSGSGTDFTLTISSLQPE | GGGGSQVQLVQSGAEVKKPGASVKVSCKASGYIFITYWMTW |
| | DFATYYCQHHYGIPFTFGQ | VRQAPGQGLEWMGQIFPASGSADYNEKFEGRVTMTTDTSTST |
| | GTKVEIKRTVAAPSVFIFPPS | AYMELRSLRSDDTAVYYCARGGGGFAYWGQGTLVTVSSAST |
| | DEQLKSGTASVVCLLNNFY | KGPSVFPLAPSSKSTSGGTAALGCLVKDYFPEPVTVSWNSGA |
| | PREAKVQWKVDNALQSGN | LTSGVHTFPAVLQSSGLYSLSSVVTVPSSSLGTQTYICNVNHK |
| | SQESVTEQDSKDSTYSLSST | PSNTKVDKKVEPKSCDKTHTCPPCPAPELLGGPSVFLFPPKPK |
| | LTLSKADYEKHKVYACEVT | DTLMISRTPEVTCVVVDVSHEDPEVKFNWYVDGVEVHNAKT |
| | HQGLSSPVTKSFNRGEC | KPREEQYNSTYRVVSVLTVLHQDWLNGKEYKCKVSNKALPA |
| | (SEQ ID NO: 16) | PIEKTISKAKGQPREPQVYTLPPSRDELTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| | | (SEQ ID NO: 56) |
| D4 | LC | HC |
| | DIQMTQSPSSLSASVGDRVT | QVQLVQSGAEVKKPGASVKVSCKASGYIFITYWMTWVRQAP |
| | ITCRTSENIYSYLAWYQQKP | GQGLEWMGQIFPASGSADYNEKFEGRVTMTTDTSTSTAYME |
| | GKAPKLLIYNAKTLAEGVPS | LRSLRSDDTAVYYCARGGGGFAYWGQGTLVTVSSASTKGPS |
| | RFSGSGSGTDFTLTISSLQPE | VFPLAPSSKSTSGGTAALGCLVKDYFPEPVTVSWNSGALTSG |
| | DFATYYCQHHYGIPFTFGQ | VHTFPAVLQSSGLYSLSSVVTVPSSSLGTQTYICNVNHKPSNT |
| | GTKVEIKRTVAAPSVFIFPPS | KVDKKVEPKSCDKTHTCPPCPAPELLGGPSVFLFPPKPKDTLM |
| | DEQLKSGTASVVCLLNNFY | ISRTPEVTCVVVDVSHEDPEVKFNWYVDGVEVHNAKTKPRE |
| | PREAKVQWKVDNALQSGN | EQYNSTYRVVSVLTVLHQDWLNGKEYKCKVSNKALPAPIEK |
| | SQESVTEQDSKDSTYSLSST | TISKAKGQPREPQVYTLPPSRDELTKNQVSLTCLVKGFYPSDI |
| | LTLSKADYEKHKVYACEVT | AVEWESNGQPENNYKTTPPVLDSDGSFFLYSKLTVDKSRWQ |
| | HQGLSSPVTKSFNRGEC | QGNVFSCSVMHEALHNHYTQKSLSLSPGKGGGGSGGGGSGG |
| | (SEQ ID NO: 16) | GGSEVQLVESGGGLVQPGGSLRLSCAASGFTFSDYWMYWVR |
| | | QAPGKGLEWVSEINTNGLITKYPDSVKGRFTISRDNAKNTLYL |
| | | QMNSLRPEDTAVYYCARSPSGFNRGQGTLVTVSS |
| | | (SEQ ID NO: 57) |

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:12 and 54, respectively. In some embodiments, the LC has at least 90%, at least 95%, at least 98%, at least 99%, or 100% sequence identity to SEQ ID NO: 12. In some embodiments, the LC has the amino acid sequence of SEQ ID NO: 12. In some embodiments, the HC has at least 90%, at least 95%, at least 98%, at least 99%, or 100% sequence identity to SEQ ID NO: 54. In some embodiments, the HC has the amino acid sequence of SEQ ID NO: 54. In some embodiments, the LC and HC each has at least 90% sequence identity to the amino acid sequences of SEQ ID NOs: 12 and 54, respectively. In some embodiments, the LC and HC each has at least 95% sequence identity to the amino acid sequences of SEQ ID NOs: 12 and 54, respectively. In some embodiments, the LC and HC each has at least 98% sequence identity to the amino acid sequences of SEQ ID NOs: 12 and 54, respectively. In some embodiments, the LC and HC each has at least 99% sequence identity to the amino acid sequences of SEQ ID NOs: 12 and 54, respectively. In some embodiments, provided herein is the bispecific antibody designated as D1, of which the LC and HC have the amino acid sequences of SEQ ID NOs: 12 and 54, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:12 and 55, respectively. In some embodiments, the LC has at least 90%, at least 95%, at least 98%, at least 99%, or 100% sequence identity to SEQ ID NO: 12. In some embodiments, the LC has the amino acid sequence of SEQ ID NO: 12. In some embodiments, the HC has at least 90%, at least 95%, at least 98%, at least 99%, or 100% sequence identity to SEQ ID NO: 55. In some embodiments, the HC has the amino acid sequence of SEQ ID NO: 55. In some embodiments, the LC and HC each has at least 90% sequence identity to the amino acid sequences of SEQ ID NOs: 12 and 55, respectively. In some embodiments, the LC and HC each has at least 95% sequence identity to the amino acid sequences of SEQ ID NOs: 12 and 55, respectively. In some embodiments, the LC and HC each has at least 98% sequence identity to the amino acid sequences of SEQ ID NOs: 12 and 55, respectively. In some embodiments, the LC and HC each has at least 99% sequence identity to the amino acid sequences of SEQ ID NOs: 12 and 55, respectively. In some embodiments, provided herein is the bispecific antibody designated as D2, of which the LC and HC have the amino acid sequences of SEQ ID NOs: 12 and 55, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs: 16 and 56, respectively. In some embodiments, the LC has at least 90%, at least 95%, at least 98%, at least 99%, or 100% sequence identity to SEQ ID NO: 16. In some embodiments, the LC has the amino acid sequence of SEQ ID NO: 16. In some embodiments, the HC has at least 90%, at least 95%, at least 98%, at least 99%, or 100% sequence identity to SEQ ID NO: 56. In some embodiments, the HC has the amino acid sequence of SEQ ID NO: 56. In some embodiments, the LC and HC each has at least 90% sequence identity to the amino acid sequences of SEQ ID NOs: 16 and 56, respectively. In some embodiments, the LC and HC each has at least 95% sequence identity to the amino acid sequences of SEQ ID NOs: 16 and 56, respectively. In some embodiments, the LC and HC each has at least 98% sequence identity to the amino acid sequences of SEQ ID NOs: 16 and 56, respectively. In some embodiments, the LC and HC each has at least 99% sequence identity to the amino acid sequences of SEQ ID NOs: 16 and 56, respectively. In some embodiments, provided herein is the bispecific antibody designated as D3, of which the LC and HC have the amino acid sequences of SEQ ID NOs: 16 and 56, respectively.

In some embodiments, provided herein are bispecific antibodies that specifically bind to human TNFα and to human IL23p19 having LC and HC, wherein the LC and HC each has at least 90%, at least 95%, at least 98%, at least 99%, or 100% sequence identity to the amino acid sequences of SEQ ID NOs:16 and 57, respectively. In some embodiments, the LC has at least 90%, at least 95%, at least 98%, at least 99%, or 100% sequence identity to SEQ ID NO: 16. In some embodiments, the LC has the amino acid sequence of SEQ ID NO: 16. In some embodiments, the HC has at least 90%, at least 95%, at least 98%, at least 99%, or 100% sequence identity to SEQ ID NO: 57. In some embodiments, the HC has the amino acid sequence of SEQ ID NO: 57. In some embodiments, the LC and HC each has at least 90% sequence identity to the amino acid sequences of SEQ ID NOs: 16 and 57, respectively. In some embodiments, the LC and HC each has at least 95% sequence identity to the amino acid sequences of SEQ ID NOs: 16 and 57, respectively. In some embodiments, the LC and HC each has at least 98% sequence identity to the amino acid sequences of SEQ ID NOs: 16 and 57, respectively. In some embodiments, the LC and HC each has at least 99% sequence identity to the amino acid sequences of SEQ ID NOs: 16 and 57, respectively. In some embodiments, provided herein is the bispecific antibody designated as D4, of which the LC and HC have the amino acid sequences of SEQ ID NOs: 16 and 57, respectively.

### 2.6 Variants

The present disclosure further contemplates additional variants and equivalents that are substantially homologous to the bispecific antibodies described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Antibodies comprising functional variants of the heavy chain, light chains, VL regions, VH regions, or one or more CDRs of the antibodies of the examples as also provided herein. A functional variant of a heavy chain, a light chain, VL, VH, or CDRs used in the context of an antibody still allows the antibody to retain at least a substantial proportion (at least about 90%, 95% or more) of functional features of the “reference” and/or “parent” antibody, including affinity and/or the specificity/selectivity, Fe inertness and PK parameters such as half-life, Tmax, Cmax. Such functional variants typically retain significant sequence identity to the parent antibody and/or have substantially similar length of heavy and light chains. Exemplary variants include those which differ from heavy and/or light chains, VH and/or VL, and/or CDR regions of the parent antibody sequences mainly by conservative substitutions, e.g., 10, such as 9, 8, 7, 6, 5, 4, 3, 2 or 1 of the substitutions in the variant may be conservative amino acid residue replacements.

In some embodiments, a variant of a bispecific antibody disclosed herein can retain its ability to bind TNFα, and IL23p19 to a similar extent, the same extent, or to a higher extent, as the parent bispecific antibody. In some embodiments, the variant can be at least about 80%, about 85%, about 90%, about 91%, about 92%, about 93%, about 94%, about 95%, about 96%, about 97%, about 98%, about 99% or more identical in amino acid sequence to the parent antibody or antigen-binding fragment. In certain embodiments, a variant of a bispecific antibody disclosed herein comprises the amino acid sequence of the parent a bispecific antibody disclosed herein with one or more conservative amino acid substitution. Conservative amino acid substitutions are known in the art and include amino acid substitutions in which one amino acid having certain physical and/or chemical properties is exchanged for another amino acid that has the same or similar chemical or physical properties.

In some embodiments, a variant of a bispecific antibody disclosed herein comprises the amino acid sequence of the parent antibody with one or more non-conservative amino acid substitutions. In some embodiments, a variant of a bispecific antibody disclosed herein comprises the amino acid sequence of the parent binding antibody with one or more non-conservative amino acid substitution, wherein the one or more non-conservative amino acid substitutions do not interfere with or inhibit one or more biological activities of the variant. In certain embodiments, the one or more conservative amino acid substitutions and/or the one or more non-conservative amino acid substitutions can enhance a biological activity of the variant, such that the biological activity of the functional variant is increased as compared to the parent antibody.

In some embodiments, the variant can have 1, 2, 3, 4, or 5 amino acid substitutions in the CDRs (e.g., VH CDR1, VH CDR2, VH CDR3, VL CDR1, VL CDR2 and VL CDR3) of the binding moiety.

In some embodiments, the bispecific antibodies provided herein include modification in their Fc regions. In some embodiments, the modified antibodies (e.g., modified Fc region) provide for altered effector functions that, in turn, affect the biological profile of the antibody. For example, in some embodiments, the deletion or inactivation (through point mutations or other means) of a constant region reduces Fe receptor binding of the modified antibody as it circulates. In some embodiments, the constant region modifications reduce the immunogenicity of the antibody. In some embodiments, the constant region modifications increase the serum half-life of the antibody. In some embodiments, the constant region modifications reduce the serum half-life of the antibody. In some embodiments, the constant region modifications decrease or remove ADCC and/or complement dependent cytotoxicity (CDC) of the antibody. In some embodiments, specific amino acid substitutions in a human IgG1 Fc region with corresponding IgG2 or IgG4 residues reduce effector functions (e.g., ADCC and CDC) in the modified antibody. In some embodiments, an antibody does not have one or more effector functions (e.g., “effectorless” antibodies). In some embodiments, the antibody has no ADCC activity and/or no CDC activity. In some embodiments, the antibody does not bind an Fc receptor and/or complement factors. In some embodiments, the antibody has no effector function(s). In some embodiments, the constant region modifications increase or enhance ADCC and/or CDC of the antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites.

In some embodiments of the bispecific antibodies provided herein, the Fc domain comprises one or more amino acid substitution that reduces binding to an Fc receptor. The Fc receptor can be a human Fc receptor. The Fc receptor can be an Fcγ receptor. The Fc receptor can be an activating Fc receptor. The Fe receptor can be an activating human Fcγ receptor, such as a human Fcγ RIIIa, Fcγ RI or Fcγ RIIa. In some embodiments of the bispecific antibodies provided herein, the Fc domain comprises one or more amino acid substitution that reduces the effector function. The effector function can be complement dependent cytotoxicity (CDC), antibody-dependent cell-mediated cytotoxicity (ADCC), antibody-dependent cellular phagocytosis (ADCP), cytokine secretion, or any combination thereof. In some embodiments, the effector function is ADCC.

In some embodiments of the bispecific antibodies provided herein, the same one or more amino acid substitution is present in each of the two subunits of the Fc region. In one aspect, the one or more amino acid substitution reduces the binding affinity of the Fc region to an Fe receptor. In one aspect, the one or more amino acid substitution reduces the binding affinity of the Fc region to an Fe receptor by at least 2-fold, at least 5-fold, or at least 10-fold.

In some embodiments, the Fc region of the bispecific antibodies provided herein include an amino acid substitution at a position selected from the group of E233, L234, L235, N297, P331 and P329. In some embodiments, the Fc region includes an amino acid substitution at a position selected from the group of L234, L235 and P329. In some embodiments, the Fc region includes the amino acid substitutions L234A and L235A. In some embodiments, the Fc region is an IgG1 Fc region, particularly a human IgG1 Fc region. In some embodiments, the Fc region includes an amino acid substitution at position P329. In some embodiments, the amino acid substitution is P329A or P329G. In some embodiments, the Fc region includes an amino acid substitution at position P329 and a further amino acid substitution at a position selected from E233, L234, L235, N297 and P331. In some embodiments, the further amino acid substitution is E233P, L234A, L235A, L235E, N297A, N297D or P331S. In some embodiments, the Fc region includes amino acid substitutions at positions P329, L234 and L235. In more particular aspects, the Fc region comprises the amino acid mutations L234A, L235A and P329G. In some embodiments, the Fc region includes the amino acid substitutions L234A, L235A and P329G. All amino acid residues are numbered according to the EU index.

In some embodiments, variants can include addition of amino acid residues at the amino- and/or carboxyl-terminal end of the antibody. The length of additional amino acids residues can range from one residue to a hundred or more residues. In some embodiments, a variant comprises an N-terminal methionyl residue. In some embodiments, a variant is engineered to be detectable and may comprise a detectable label and/or protein (e.g., a fluorescent tag or an enzyme).

The variant antibodies described herein can be generated using methods known in the art, including but not limited to, site-directed mutagenesis, alanine scanning mutagenesis, and PCR mutagenesis.

In some embodiments, bispecific antibodies disclosed herein can be chemically modified naturally or by intervention. In some embodiments, the bispecific antibodies are chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications can be carried out by known techniques. The bispecific antibodies provided herein can comprise one or more analogs of an amino acid (including, for example, unnatural amino acids), as well as other modifications known in the art.

The bispecific antibodies of the present disclosure can be analyzed for their physical, chemical and/or biological properties by various methods known in the art. In some embodiments, a bispecific antibody provided herein is tested for its ability to bind human TNFα and/or human IL23p19. Binding assays include, but are not limited to, BLI, SPR (e.g., Biacore), ELISA, and FACS. In addition, antibodies can be evaluated for solubility, stability, thermostability, viscosity, expression levels, expression quality, and/or purification efficiency.

In some embodiments, bispecific antibodies disclosed herein can be conjugated to a detectable substance or molecule that allows the agent to be used for detection. A detectable substance can include, but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³I, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷Sc, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions positron emitting metals; and magnetic metal ions.

The anti-TNFα/IL23p19 bispecific antibodies disclosed herein can be attached to a solid support. Such solid supports include, but are not limited to, glass, cellulose, polyacrylamide, nylon, polystyrene, polyvinyl chloride, or polypropylene. In some embodiments, an immobilized bispecific antibody is used in an immunoassay. In some embodiments, an immobilized bispecific antibody is used in purification.

### 3. Polynucleotides, Vectors, and Cells

Provided herein are polynucleotides encoding at least one light chain or one heavy chain of the anti-TNFα/IL23p19 bispecific antibodies disclosed herein. In some embodiments, the polynucleotides provided herein encode one polypeptide, such as a light chain or a heavy chain of a bispecific antibody. In some embodiments, the polynucleotides provided herein encode more than one polypeptide. In some embodiments, the polynucleotides provided herein can encode, for example, the light chain and heavy chain of a bispecific antibody provided herein, respectively. Cistrons can be separated by, for example, an internal ribosomal entry site (IRES) or 2A element. An IRES, as understood in the art, refers to nucleotide sequences in an expression cassette which when transcribed into mRNA, can recruit ribosomes directly, without a previous scanning of untranslated region of mRNA by the ribosomes. A 2A element, as understood in the art, encoding self-cleaving short peptides (about 20 amino acids) that provide a mechanism for subsequent separation of equimolarly produced polypeptides of interest. Illustrative 2A self-cleaving peptides include P2A (SEQ ID NO: 76), E2A (SEQ ID NO: 77), F2A (SEQ ID NO: 78), and T2A (SEQ ID NO: 79).

In some embodiments, provided herein are polynucleotides encoding the LC1, the HC1, the LC2, the HC2, or any combination thereof, of the anti-TNFα/IL23p19 bispecific antibodies disclosed herein having the CrossMab-KIH structure. In some embodiments, provided herein are polynucleotides encoding the LC, the HC, or both of the anti-TNFα/IL23p19 bispecific antibodies disclosed herein having the IgG-ScFv structure, the DVD-Ig structure, or the SMAB structure.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

In some embodiments, provided herein are polynucleotides encoding the LC1, the HC1, the LC2, the HC2, or any combination thereof, of the bispecific antibody designated as A1. In some embodiments, provided herein are a plurality of polynucleotides that collectively encode the LC1, the HC1, the LC2, and the HC2 of the bispecific antibody designated as A1. In some embodiments, provided herein are polynucleotides encoding the LC1, the HC1, the LC2, the HC2, or any combination thereof, of the bispecific antibody designated as A2. In some embodiments, provided herein are a plurality of polynucleotides that collectively encode the LC1, the HC1, the LC2, and the HC2 of the bispecific antibody designated as A2. In some embodiments, provided herein are polynucleotides encoding the LC1, the HC1, the LC2, the HC2, or any combination thereof, of the bispecific antibody designated as A3. In some embodiments, provided herein are a plurality of polynucleotides that collectively encode the LC1, the HC1, the LC2, and the HC2 of the bispecific antibody designated as A3. In some embodiments, provided herein are polynucleotides encoding the LC1, the HC1, the LC2, the HC2, or any combination thereof, of the bispecific antibody designated as A4. In some embodiments, provided herein are a plurality of polynucleotides that collectively encode the LC1, the HC1, the LC2, and the HC2 of the bispecific antibody designated as A4. In some embodiments, provided herein are polynucleotides encoding the LC1, the HC1, the LC2, the HC2, or any combination thereof, of the bispecific antibody designated as A5. In some embodiments, provided herein are a plurality of polynucleotides that collectively encode the LC1, the HC1, the LC2, and the HC2 of the bispecific antibody designated as A5. In some embodiments, provided herein are polynucleotides encoding the LC1, the HC1, the LC2, the HC2, or any combination thereof, of the bispecific antibody designated as A6. In some embodiments, provided herein are a plurality of polynucleotides that collectively encode the LC1, the HC1, the LC2, and the HC2 of the bispecific antibody designated as A6. In some embodiments, provided herein are polynucleotides encoding the LC1, the HC1, the LC2, the HC2, or any combination thereof, of the bispecific antibody designated as A7. In some embodiments, provided herein are a plurality of polynucleotides that collectively encode the LC1, the HC1, the LC2, and the HC2 of the bispecific antibody designated as A7. In some embodiments, provided herein are polynucleotides encoding the LC1, the HC1, the LC2, the HC2, or any combination thereof, of the bispecific antibody designated as A8. In some embodiments, provided herein are a plurality of polynucleotides that collectively encode the LC1, the HC1, the LC2, and the HC2 of the bispecific antibody designated as A8.

In some embodiments, provided herein are polynucleotides encoding the LC, the HC, or both, of the bispecific antibody designated as B1. In some embodiments, provided herein are a first and a second polynucleotides that encode the LC and HC of the bispecific antibody designated as B1, respectively. In some embodiments, provided herein are polynucleotides encoding the LC, the HC, or both, of the bispecific antibody designated as B2. In some embodiments, provided herein are a first and a second polynucleotides that encode the LC and HC of the bispecific antibody designated as B2, respectively. In some embodiments, provided herein are polynucleotides encoding the LC, the HC, or both, of the bispecific antibody designated as B3. In some embodiments, provided herein are a first and a second polynucleotides that encode the LC and HC of the bispecific antibody designated as B3, respectively. In some embodiments, provided herein are polynucleotides encoding the LC, the HC, or both, of the bispecific antibody designated as B4. In some embodiments, provided herein are a first and a second polynucleotides that encode the LC and HC of the bispecific antibody designated as B4, respectively. In some embodiments, provided herein are polynucleotides encoding the LC, the HC, or both, of the bispecific antibody designated as B5. In some embodiments, provided herein are a first and a second polynucleotides that encode the LC and HC of the bispecific antibody designated as B5, respectively. In some embodiments, provided herein are polynucleotides encoding the LC, the HC, or both, of the bispecific antibody designated as B6. In some embodiments, provided herein are a first and a second polynucleotides that encode the LC and HC of the bispecific antibody designated as B6, respectively. In some embodiments, provided herein are polynucleotides encoding the LC, the HC, or both, of the bispecific antibody designated as B7. In some embodiments, provided herein are a first and a second polynucleotides that encode the LC and HC of the bispecific antibody designated as B7, respectively. In some embodiments, provided herein are polynucleotides encoding the LC, the HC, or both, of the bispecific antibody designated as B8. In some embodiments, provided herein are a first and a second polynucleotides that encode the LC and HC of the bispecific antibody designated as B8, respectively.

In some embodiments, provided herein are polynucleotides encoding the LC, the HC, or both, of the bispecific antibody designated as C1. In some embodiments, provided herein are a first and a second polynucleotides that encode the LC and HC of the bispecific antibody designated as C1, respectively. In some embodiments, provided herein are polynucleotides encoding the LC, the HC, or both, of the bispecific antibody designated as C2. In some embodiments, provided herein are a first and a second polynucleotides that encode the LC and HC of the bispecific antibody designated as C2, respectively. In some embodiments, provided herein are polynucleotides encoding the LC, the HC, or both, of the bispecific antibody designated as C3. In some embodiments, provided herein are a first and a second polynucleotides that encode the LC and HC of the bispecific antibody designated as C3, respectively. In some embodiments, provided herein are polynucleotides encoding the LC, the HC, or both, of the bispecific antibody designated as C4. In some embodiments, provided herein are a first and a second polynucleotides that encode the LC and HC of the bispecific antibody designated as C4, respectively. In some embodiments, provided herein are polynucleotides encoding the LC, the HC, or both, of the bispecific antibody designated as C5. In some embodiments, provided herein are a first and a second polynucleotides that encode the LC and HC of the bispecific antibody designated as C5, respectively. In some embodiments, provided herein are polynucleotides encoding the LC, the HC, or both, of the bispecific antibody designated as C6. In some embodiments, provided herein are a first and a second polynucleotides that encode the LC and HC of the bispecific antibody designated as C6, respectively. In some embodiments, provided herein are polynucleotides encoding the LC, the HC, or both, of the bispecific antibody designated as C7. In some embodiments, provided herein are a first and a second polynucleotides that encode the LC and HC of the bispecific antibody designated as C7, respectively. In some embodiments, provided herein are polynucleotides encoding the LC, the HC, or both, of the bispecific antibody designated as C8. In some embodiments, provided herein are a first and a second polynucleotides that encode the LC and HC of the bispecific antibody designated as C8, respectively.

In some embodiments, provided herein are polynucleotides encoding the LC, the HC, or both, of the bispecific antibody designated as D1. In some embodiments, provided herein are a first and a second polynucleotides that encode the LC and HC of the bispecific antibody designated as D1, respectively. In some embodiments, provided herein are polynucleotides encoding the LC, the HC, or both, of the bispecific antibody designated as D2. In some embodiments, provided herein are a first and a second polynucleotides that encode the LC and HC of the bispecific antibody designated as D2, respectively. In some embodiments, provided herein are polynucleotides encoding the LC, the HC, or both, of the bispecific antibody designated as D3. In some embodiments, provided herein are a first and a second polynucleotides that encode the LC and HC of the bispecific antibody designated as D3, respectively. In some embodiments, provided herein are polynucleotides encoding the LC, the HC, or both, of the bispecific antibody designated as D4. In some embodiments, provided herein are a first and a second polynucleotides that encode the LC and HC of the bispecific antibody designated as D4, respectively.

The term “polynucleotide that encode a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA can be cDNA, genomic DNA, or synthetic DNA, and can be double-stranded or single-stranded. Single stranded DNA can be the coding strand or non-coding (anti-sense) strand. The polynucleotides of the disclosure can be mRNA.

The present disclosure also provides variants of the polynucleotides described herein, wherein the variants have a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding at least one polypeptide chain of an anti-TNFα/IL23p19 bispecific antibody described herein. As used herein, the phrase “a polynucleotide having a nucleotide sequence at least about 95% identical to a polynucleotide sequence” means that the nucleotide sequence of the polynucleotide is identical to a reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises the coding sequence for a polypeptide (e.g., an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., an antibody) fused in the same reading frame to a marker or tag sequence. For example, in some embodiments, a marker sequence is a hexa-histidine tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker can be used in conjunction with other markers or tags.

In some embodiments, a polynucleotide is isolated. In some embodiments, a polynucleotide is substantially pure.

In some embodiments, provided herein are also vectors comprising a polynucleotide disclosed herein. The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product, and it is further understood that expression levels can be optimized to obtain sufficient expression using methods well known in the art.

In some embodiments, vectors provided herein can be expression vectors. In some embodiments, vectors provided herein comprise a polynucleotide encoding at least one polypeptide chain of the anti-TNFα/IL23p19 bispecific antibodies described herein. In some embodiments, provided herein are recombinant expression vectors, which can be used to amplify and express a polynucleotide encoding at least one polypeptide chain of the anti-TNFα/IL23p19 bispecific antibodies described herein. For example, a recombinant expression vector can be a replicable DNA construct that includes synthetic or cDNA-derived DNA fragments encoding at least one polypeptide chain of the anti-TNFα/IL23p19 bispecific antibodies described herein, operatively linked to suitable transcriptional and/or translational regulatory elements derived from mammalian, microbial, viral or insect genes. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in certain expression systems include a leader sequence enabling extracellular secretion of translated protein by a host cell. In some embodiments, in situations where recombinant protein is expressed without a leader or transport sequence, a polypeptide can include an N-terminal methionine residue.

Examples of vectors are plasmid, autonomously replicating sequences, and transposable elements. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. Useful expression vectors for eukaryotic hosts include, for example, vectors comprising expression control sequences from SV40, bovine papilloma virus, adenovirus, and cytomegalovirus. Exemplary transposon systems such as Sleeping Beauty and PiggyBac can be used, which can be stably integrated into the genome (e.g., Ivics et al., Cell, 91 (4): 501-510 (1997); Cadiinanos et al., (2007) Nucleic Acids Research. 35 (12): e87).

In some embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In some embodiments, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

“Expression control sequences,” “control elements,” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements can vary in their strength and specificity. Depending on the vector system and host utilized, any number of suitable transcription and translation elements, including ubiquitous promoters and inducible promoters can be used.

Illustrative ubiquitous expression control sequences that can be used in present disclosure include, but are not limited to, a cytomegalovirus (CMV) immediate early promoter, a viral simian virus 40 (SV40) promoter (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, and a β-actin promoter.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc. The bispecific antibodies described herein can be produced by any method known in the art, including chemical synthesis and recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) MOLECULAR CLONING: A LABORATORY MANUAL, Cold Spring Harbor Laboratory Press; Sambrook et al. (1989), MOLECULAR CLONING: A LABORATORY MANUAL, Second Edition, Cold Spring Harbor Laboratory Press; Sambrook et al. (2001) MOLECULAR CLONING: A LABORATORY MANUAL, Cold Spring Harbor Laboratory Press, Cold Spring Harbor, NY; Ausubel et al., CURRENT PROTOCOLS IN MOLECULAR BIOLOGY, John Wiley & Sons (1987 and annual updates); CURRENT PROTOCOLS IN IMMUNOLOGY, John Wiley & Sons (1987 and annual updates) Gait (ed.) (1984) OLIGONUCLEOTIDE SYNTHESIS: A PRACTICAL APPROACH, IRL Press; Eckstein (ed.) (1991) OLIGONUCLEOTIDES AND ANALOGUES: A PRACTICAL APPROACH, IRL Press; Birren et al. (eds.) (1999) GENOME ANALYSIS: A LABORATORY MANUAL, Cold Spring Harbor Laboratory Press; Borrebaeck (ed.) (1995) ANTIBODY ENGINEERING, Second Edition, Oxford University Press; Lo (ed.) (2006) ANTIBODY ENGINEERING: METHODS AND PROTOCOLS (METHODS IN MOLECULAR BIOLOGY); Vol. 248, Humana Press, Inc; each of which is incorporated herein by reference in its entirety.

The present disclosure also provides cells comprising the polynucleotides disclosed herein that encode at least one polypeptide chain of the anti-TNFα/IL23p19 bispecific antibodies described herein. In some embodiments, cells provided herein comprise a polynucleotide that encodes the LC1, the HC1, the LC2, and the HC2 of the anti-TNFα/IL23p19 bispecific antibodies disclosed herein having the CrossMab-KIH structure. In some embodiments, cells provided herein comprise a plurality of the polynucleotides that collectively encode the LC1, the LC2, the HC1, and the HC2 of the anti-TNFα/IL23p19 bispecific antibodies disclosed herein having the CrossMab-KIH structure.

In some embodiments, cells provided herein comprise a polynucleotide that encodes both the LC and the HC of the anti-TNFα/IL23p19 bispecific antibodies disclosed herein having the IgG-scFv structure, the DVID-Ig, or the SMAB structure. In some embodiments, cells provided herein comprise a first polynucleotide that encodes the LC and a second polynucleotide that encodes HC of the anti-TNFα/IL23p19 bispecific antibodies disclosed herein having the IgG-scFv structure, the DVID-Ig, or the SMAB structure.

Cells comprising vectors disclosed herein are also contemplated. In some embodiments, provided herein are host cells comprising a vector comprising a polynucleotide disclosed herein. In some embodiments, host cells provided herein comprise a vector or multiple vectors that collectively comprise the polynucleotides encoding the polypeptide chains of the anti-TNFα/IL23p19 bispecific antibodies described herein. In some embodiments, host cells provided herein produce the anti-TNFα/IL23p19 bispecific antibodies described herein.

Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

### 4. Methods of Production

Provided herein are also methods of producing the anti-TNFα/IL23p19 bispecific antibodies disclosed herein. In some embodiments, the bispecific antibodies disclosed herein are comprised of more than one polypeptide chain, which can be produced separately or together. In some embodiments, methods provided herein produce at least one polypeptide chain of the bispecific antibodies disclosed herein. In some embodiments, methods provided herein produce all polypeptide chains of the bispecific antibodies disclosed herein.

The bispecific antibodies or polypeptides described herein can be produced and isolated using methods known in the art. Polypeptides can be synthesized, in whole or in part, using chemical methods (see, e.g., Caruthers (1980). Nucleic Acids Res. Symp. Ser. 215; Horn (1980); and Banga, A. K., THERAPEUTIC PEPTIDES AND PROTEINS, FORMULATION, PROCESSING AND DELIVERY SYSTEMS (1995) Technomic Publishing Co., Lancaster, PA). Peptide synthesis can be performed using various solid phase techniques (see, e.g., Roberge, Science 269:202 (1995); Merrifield, Methods. Enzymol. 289:3 (1997)) and automated synthesis may be achieved, e.g., using the ABI 431A Peptide Synthesizer (Perkin Elmer) in accordance with the manufacturer's instructions. Peptides can also be synthesized using combinatorial methodologies. Synthetic residues and polypeptides can be synthesized using a variety of procedures and methodologies known in the art (see, e.g., ORGANIC SYNTHESES COLLECTIVE VOLUMES, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London Ser A 317:415 (1986)) and other techniques can be performed on cloned DNA to produce invention peptide sequences, variants, fusions and chimeras, and variations, derivatives, substitutions and modifications thereof.

A variety of host-expression vector systems can be utilized to recombinantly express the bispecific antibodies described herein or one or more of their polypeptide chains. Suitable host cells for expression include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art. Such host-expression systems represent vehicles by which the coding sequences of the bispecific antibodies described herein can be produced and subsequently purified, but also represent cells which may, when transformed or transfected with the appropriate polynucleotide coding sequences, express the bispecific antibodies described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the compounds described herein; yeast (e.g., Saccharomyces pichia) transformed with recombinant yeast expression vectors containing sequences encoding the compounds described herein; insect cell systems infected with recombinant virus expression vectors (e.g., baclovirus) containing the sequences encoding the compounds described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the molecules compounds described herein; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, many expression vectors can be advantageously selected depending upon the use intended for the protein being expressed. For example, when a large quantity of such a protein is to be produced, for the generation of pharmaceutical compositions of the bispecific antibodies described herein, vectors which direct the expression of high levels of protein products that are readily purified can be desirable. Such vectors include, but are not limited, to the E. coli expression vector pUR278 (Ruther et al. (1983), EMBO J. 2: 1791-1794); pIN vectors (Inouye et al. (1985), Nucleic Acids Res. 13:3101-3110; Van Heeke et al. (1989), J. Biol. Chem. 24:5503-5509); and the like. pGEX vectors can also be used to express polypeptides as fusion proteins with glutathione S-transferase (GST). In general, such proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

Useful expression vectors for eukaryotic hosts include, for example, vectors comprising expression control sequences from SV40, bovine papilloma virus, adenovirus, and cytomegalovirus. In mammalian host cells, a number of viral-based expression systems can be utilized. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art. Autographa californica nuclear polyhedrosis virus (AcNPV) is used as a vector to express foreign genes.

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. For example, in certain embodiments, the antibodies described herein can be expressed as a single gene product (e.g., as a single polypeptide chain, i.e., as a polyprotein precursor), requiring proteolytic cleavage by native or recombinant cellular mechanisms to form separate polypeptides of the bispecific antibodies described herein. The disclosure thus encompasses engineering a nucleic acid sequence to encode a polyprotein precursor molecule comprising the polypeptides of the bispecific antibodies described herein, which includes coding sequences capable of directing post translational cleavage of said polyprotein precursor. Post-translational cleavage of the polyprotein precursor results in the polypeptides of the bispecific antibodies described herein. The post translational cleavage of the precursor molecule comprising the polypeptides of the compounds described herein can occur in vivo (i.e., within the host cell by native or recombinant cell systems/mechanisms, e.g. furin cleavage at an appropriate site) or can occur in vitro (e.g. incubation of said polypeptide chain in a composition comprising proteases or peptidases of known activity and/or in a composition comprising conditions or reagents known to foster the desired proteolytic action). Purification and modification of recombinant proteins is well known in the art such that the design of the polyprotein precursor can include a number of embodiments readily appreciated by a skilled artisan. Any known proteases or peptidases known in the art can be used for the described modification of the precursor molecule.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express compounds described herein can be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the compounds described herein. Such engineered cell lines may be particularly useful in screening and evaluation of compounds that interact directly or indirectly with the compounds described herein.

A number of selection systems may be used, including but not limited to the herpes simplex virus thymidine kinase (Wigler et al. (1977), Cell 11: 223-232), hypoxanthine-guanine phosphoribosyltransferase (Szybalska et al. (1992) Bioessays 14: 495-500), and adenine phosphoribosyltransferase (Lowy et al. (1980), Cell 22: 817-823) genes can be employed in tk-, hgprt- or aprt-cells, respectively. Also, antimetabolite resistance can be used as the basis of selection for the following genes: dhfr, which confers resistance to methotrexate (Wigler et al. (1980) PNAS 77:3567-3570; O'Hare et al. (1981) PNAS, 78: 1527-1531); gpt, which confers resistance to mycophenolic acid (Mulligan et al. (1981) PNAS, 78: 2072-2076); neo, which confers resistance to the aminoglycoside G-418 (Tolstoshev (1993), Ann. Rev. Pharmacol. Toxicol. 32:573-596; Mulligan (1993), Science 260:926-932; and Morgan et al. (1993), Ann. Rev. Biochem. 62: 191-217) and hygro, which confers resistance to hygromycin (Santerre et al. (1984) Gene 30: 147-156). Methods commonly known in the art of recombinant DNA technology which can be used are described in Ausubel et al. (eds.), 1993, CURRENT PROTOCOLS IN MOLECULAR BIOLOGY, John Wiley & Sons, NY; Kriegler, 1990, GENE TRANSFER AND EXPRESSION, A LABORATORY MANUAL, Stockton Press, NY; and in Chapters 12 and 13, Dracopoli et al. (eds), 1994, CURRENT PROTOCOLS IN HUMAN GENETICS, John Wiley & Sons, NY.

The expression levels of bispecific antibodies described herein or their polypeptide chains can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system described herein is amplifiable, increase in the level of inhibitor present in culture of host cell will increase the number of copies of the marker gene. Since the amplified region is associated with the nucleotide sequence of a protein of interest, production of the protein of interest will also increase (Crouse et al. (1983) Mol. Cell. Biol. 3:257-266).

The host cell can be co-transfected with more than one expression vectors, each encoding a polypeptide chain of a bispecific antibody described herein. The vectors can contain identical selectable markers which enable equal expression of all polypeptides. Alternatively, a single vector can be used which encodes two or more polypeptides. The coding sequences for the polypeptides of compounds described herein can comprise cDNA or genomic DNA.

Once a bispecific antibody described herein or polypeptide described herein has been recombinantly expressed, it can be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (optionally after Protein A selection where the compound comprises an Fc domain (or portion thereof)), and sizing column chromatography), centrifugation, differential solubility, or by any other standard technique for the purification of polypeptides or antibodies.

Provided herein are methods of producing an anti-TNFα/IL23p19 bispecific antibody described herein or a polypeptide chain of a bispecific antibody described herein, the method comprising obtaining a cell described herein and expressing the polynucleotide described herein in said cell. In some embodiments, the method further comprises isolating and purifying a bispecific antibody or polypeptide chain described herein.

The bispecific antibodies described herein can be tested for binding to human TNFα and/or IL23p19 by, for example, standard ELISA. Briefly, microtiter plates are coated with purified antigen, and then blocked with bovine serum albumin. Dilutions of antibody are added to each well and incubated. The plates are washed and incubated with secondary reagent (e.g., for human antibodies, a goat-anti-human IgG Fc-specific polyclonal reagent) conjugated to horseradish peroxidase (HRP).

After washing, the plates can be developed and analyzed by a spectrophotometer. Antibodies can be further tested by flow cytometry for binding to a cell line expressing human TNFα and/or IL23p19, but not to a control cell line that does not express the target antigen. Briefly, the binding of antibodies can be assessed by incubating TNFα and/or IL23p19 expressing CHO cells with the bispecific antibody provided herein. The cells can be washed, and binding can be detected with an anti-human IgG Ab. Flow cytometric analyses can be performed using a FACS can flow cytometry (Becton Dickinson, San Jose, CA).

The anti-TNFα/IL23p19 bispecific antibodies provided herein can be further tested for reactivity with the target antigen(s) by Western blotting, and other methods known in the art for analyzing binding affinity, cross-reactivity, and binding kinetics of various anti-TNFα/IL23p19 bispecific antibodies described herein include, for example, biolayer interferometry (BLI) using, for example, Gator system (Probe Life) or the Octet-96 system (Sartorius AG), or BIACORE™ surface plasmon resonance (SPR) analysis using a BIACORE™ 2000 SPR instrument (Biacore AB, Uppsala, Sweden).

### 5. Pharmaceutical Compositions

Provided herein are also pharmaceutical compositions comprising the anti-TNFα/IL23p19 bispecific antibodies disclosed herein. In some embodiments, the pharmaceutical composition comprises a therapeutically effective amount of the bispecific antibodies disclosed herein and a pharmaceutically acceptable carrier. In some embodiments, the pharmaceutical compositions are useful in treating an inflammatory disease or an autoimmune disease.

The term “pharmaceutically acceptable carrier” or “pharmaceutically acceptable excipient” refers to a material that is suitable for drug administration to an individual along with an active agent without causing undesirable biological effects or interacting in a deleterious manner with any of the other components of the pharmaceutical composition. In some embodiments, the pharmaceutical compositions disclosed herein can comprise one or more of a buffer system, a preservative, a tonicity agent, a chelating agent, a stabilizer and/or a surfactant, as well as various combinations thereof. The use of preservatives, isotonic agents, chelating agents, stabilizers and surfactants in pharmaceutical compositions is well-known to the skilled person. Reference may be made to REMINGTON: THE SCIENCE AND PRACTICE OF PHARMACY, 19^{th} edition, 1995.

In some embodiments, the pharmaceutical compositions provided herein comprise the anti-TNFα/IL23p19 bispecific antibodies provided herein. The anti-TNFα/IL23p19 bispecific antibodies can be present at various concentrations. In some embodiments, the pharmaceutical compositions provided herein comprise the anti-TNFα/IL23p19 bispecific antibodies provided herein at 1-1000 mg/mL. In some embodiments, the pharmaceutical compositions comprise the anti-TNFα/IL23p19 bispecific antibodies provided herein at 10-500 mg/mL, 10-400 mg/mL, 10-300 mg/mL, 10-200 mg/mL, 10-100 mg/mL, 20-100 mg/mL, or 50-100 mg/mL. In some embodiments, the pharmaceutical compositions provided herein comprise the anti-TNFα/IL23p19 bispecific antibodies provided herein at about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 120 mg/mL, about 150 mg/mL, about 180 mg/mL, about 200 mg/mL, about 300 mg/mL, about 500 mg/mL, about 800 mg/mL, or about 1000 mg/mL. Dosages can be readily adjusted by those skilled in the art; for example, a decrease in purity may require an increase in dosage.

Pharmaceutically acceptable carriers that can be used in compositions provided herein include any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active ingredient (i.e., the anti-TNFα/IL23p19 bispecific antibodies) can be coated in a material to protect the active ingredient from the action of acids and other natural conditions that can inactivate the active ingredient.

Provided herein are also pharmaceutical compositions or formulations that improve the stability of the anti-TNFα/IL23p19 bispecific antibodies to allow for their long-term storage. In some embodiments, the pharmaceutical composition or formulation disclosed herein comprises: (a) the anti-TNFα/IL23p19 bispecific antibodies disclosed herein; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C.

Buffering agents useful in the pharmaceutical compositions or formulations disclosed herein can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity and other properties can also depend on the pH of the formulation. Common buffering agents include, but are not limited to, histidine, citrate, succinate, acetate and phosphate. In some embodiments, a buffering agent comprises histidine (e.g., L-histidine) with isotonicity agents and potentially pH adjustment with an acid or a base known in the art. In certain embodiments, the buffering agent is L-histidine. In certain embodiments, the pH of the formulation is maintained between about 2 and about 10, or between about 4 and about 8.

Stabilizing agents are added to a pharmaceutical product to stabilize that product. Such agents can stabilize proteins in different ways. Common stabilizing agents include, but are not limited to, amino acids such as glycine, alanine, lysine, arginine, or threonine, carbohydrates such as glucose, sucrose, trehalose, rafftnose, or maltose, polyols such as glycerol, mannitol, sorbitol, cyclodextrins or destrans of any kind and molecular weight, or PEG. In some embodiments, the stabilizing agent is chosen to maximize the stability of antibodies in lyophilized preparations. In certain embodiments, the stabilizing agent is sucrose and/or arginine.

Bulking agents can be added to a pharmaceutical composition or formulation to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In some embodiments, the pharmaceutical compositions disclosed herein are freeze-dried, to which the physician or the patient adds solvents and/or diluents prior to use.

Pharmaceutical compositions disclosed herein can also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. In some embodiments, provided herein is a pharmaceutical composition comprising the anti-TNFα/IL23p19 bispecific antibodies or cells provided herein wherein the composition is suitable for local administration.

Pharmaceutical compositions or formulations typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The amount of active ingredient which can be combined with a carrier material in the pharmaceutical compositions or formulations disclosed herein can vary. In some embodiments, the amount of active ingredient which can be combined with a carrier material is the amount that produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

The pharmaceutical compositions disclosed herein can be prepared with carriers that protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known to those skilled in the art. See. e.g., SUSTAINED AND CONTROLLED RELEASE DRUG DELIVERY SYSTEMS, J. R. Robinson, ed., Marcel Dekker, Inc., New York, 1978.

Provided herein are also kits for preparation of pharmaceutical compositions having the anti-TNFα/IL23p19 bispecific antibodies disclosed herein. In some embodiments, the kit comprises the anti-TNFα/IL23p19 bispecific antibodies disclosed herein and a pharmaceutically acceptable carrier in one or more containers. In another embodiment, the kits can comprise the anti-TNFα/IL23p19 bispecific antibodies disclosed herein for administration to a subject. In specific embodiments, the kits comprise instructions regarding the preparation and/or administration of the anti-TNFα/IL23p19 bispecific antibodies.

### 6. Methods and Uses

The anti-TNFα/IL23p19 bispecific antibodies provided herein can be used in medical treatment. Provided herein are also methods of reducing TNFα and/or IL23p19-associated autoimmunity or inflammation in a subject in need thereof comprising administering to the subject a therapeutically effective amount of the anti-TNFα/IL23p19 bispecific antibodies disclosed herein.

Provided herein are also methods of treating an autoimmune disease in a subject in need thereof comprising administering to the subject a therapeutically effective amount of the anti-TNFα/IL23p19 bispecific antibodies disclosed herein. Provided herein are also methods of treating an inflammatory disease comprising administering to the subject in a subject in need thereof a therapeutically effective amount of the anti-TNFα/IL23p19 bispecific antibodies disclosed herein. In some embodiments, the subject is a human. In some embodiments, the subject has an autoimmune disease. In some embodiments, the subject has an inflammatory disease. In some embodiments, the subject is at risk of developing an autoimmune disease. In some embodiments, the subject is at risk of developing an inflammatory disease.

Provided herein are uses of the anti-TNFα/IL23p19 bispecific antibodies disclosed herein as a medicament. Provided herein are also uses of the anti-TNFα/IL23p19 bispecific antibodies disclosed herein in treating an autoimmune disease. Provided herein are also uses of the anti-TNFα/IL23p19 bispecific antibodies disclosed herein in treating an inflammatory disease. Provided herein are uses of the anti-TNFα/IL23p19 bispecific antibodies disclosed herein for the preparation of a medicament for treating an autoimmune disease. Provided herein are also uses of the anti-TNFα/IL23p19 bispecific antibodies disclosed herein for the preparation of a medicament for treating an inflammatory disease.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “subject” as used herein refers to any animal (e.g., a mammal), including, but not limited to, humans, non-human primates, canines, felines, rodents, and the like, which is to be the recipient of a particular treatment. Mammals include, but are not limited to, farm animals, sport animals, pets, primates, horses, dogs, cats, mice and rats. A human subject who needs the treatment may be a human subject having, at risk for, or suspected of having a disease. A subject having a disease can be identified by routine medical examination, e.g., a physical examination, a laboratory test, an organ functional test, a CT scan, or an ultrasound. A subject suspected of having any of such a disease can show one or more symptoms of the disease. Signs and symptoms for diseases, e.g., autoimmune and inflammatory diseases, are well known to those of ordinary skill in the art. A subject at risk for the disease can be a subject having one or more of the risk factors for that disease. A subject can be a human. A subject can have a particular disease or condition.

Non-limiting examples of autoimmune diseases include rheumatoid arthritis, psoriasis, type 1 diabetes, systemic lupus erythematosus, transplant rejection, autoimmune thyroid disease (Hashimoto's disease), sarcoidosis, scleroderma, granulomatous vasculitis, Crohn's disease, ulcerative colitis, Sjogren's disease, ankylosing spondylitis, psoriatic arthritis, polymyositis dermatomyositis, polyarteritis nodosa, immunologically mediated blistering skin diseases, Behcet's syndrome, multiple sclerosis, systemic sclerosis, hidradenitis suppurativa, palmoplantar pustulosis, Pityriasis rubra pilaris, atopic dermatitis, juvenile idiopathic arthritis, Goodpasture's disease or immune mediated glomerulonephritis.

Non-limiting examples of inflammatory diseases include including rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (ALPS), autoimmune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyneuropathy, cicatricial pemphigoid, cold agglutinin disease, Crest syndrome, Crohn's disease, Dego's disease, dermatomyasitis, dermatomyositis-juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, inflammatory myopathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis. In some embodiments, the autoimmune or inflammatory disease is Crohn's disease, ankylosing spondylitis, or psoriatic arthritis.

In some embodiments, the bispecific antibodies and pharmaceutical compositions provided herein can be used for treating an autoimmune disease or an inflammatory disease that is plaque psoriasis, hidradenitis suppurativa, palmoplantar pustulosis, pityriasis rubra pilaris, atopic dermatitis, systemic sclerosis, takayasu arteritis, giant cell arteritis, uveitis, rheumatoid arthritis, psoriatic arthritis, juvenile idiopathic arthritis, ankylosing spondylitis, Crohn's disease, ulcerative colitis, intestinal Behcet's disease, or inflammatory myopathy. In some embodiments, the autoimmune disease or inflammatory disease is plaque psoriasis. In some embodiments, the autoimmune disease or inflammatory disease is psoriatic arthritis. In some embodiments, the autoimmune disease or inflammatory disease is Crohn's disease. In some embodiments, the autoimmune disease or inflammatory disease is ulcerative colitis. In some embodiments, the autoimmune disease or inflammatory disease is palmoplantar pustulosis.

In the methods and uses disclosed herein, a therapeutically effective amount of anti-TNFα/IL23p19 bispecific antibodies or pharmaceutical compositions disclosed herein is administered to a subject that can benefit from reduction in autoimmunity. The subject can have unwanted, unregulated, or excessive autoimmune activation. The subject can be at risk of developing unwanted, unregulated, or excessive autoimmune activation. Actual dosage levels of the active ingredients (i.e., the anti-TNFα/IL23p19 bispecific antibodies disclosed herein) in the pharmaceutical compositions described herein can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

The anti-TNFα/IL23p19 bispecific antibodies disclosed herein can be administered as a sustained release formulation, in which case less frequent administration is required. Dosage and frequency vary depending on the half-life of the anti-TNFα/IL23p19 bispecific antibodies in the patient. In therapeutic applications, a relatively high dosage at relatively short intervals is sometimes required until progression of the disease is reduced or terminated, and until the patient shows partial or complete amelioration of symptoms of disease.

The anti-TNFα/IL23p19 bispecific antibodies or pharmaceutical compositions provided herein can be administered to a subject by any methods known in the art, including, but not limited to, pleural administration, intravenous administration, subcutaneous administration, intranodal administration, intramuscular administration, intradermal administration, intrathecal administration, intrapleural administration, intraperitoneal administration, intracranial administration, spinal or other parenteral routes of administration, for example by injection or infusion, or direct administration to the thymus. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. In some embodiments, subcutaneous administration is adopted. In some embodiments, intravenous administration is adopted. In some embodiments, oral administration is adopted. In one embodiment, the antibodies or antigen-binding fragments provided herein can be delivered locally. In another embodiment, the antibodies or antigen-binding fragments provided herein can be administered systemically.

Anti-TNFα/IL23p19 bispecific antibodies or pharmaceutical compositions provided herein can be administered with medical devices known in the art. For example, in some embodiments, a needleless hypodermic injection device can be used, such as the devices disclosed in U.S. Pat. Nos. 5,399,163; 5,383,851; 5,312,335; 5,064,413; 4,941,880; 4,790,824; or 4,596,556. Examples of well-known implants and modules for use described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In some embodiments, the anti-TNFα/IL23p19 bispecific antibodies or pharmaceutical compositions provided herein can be administered with an additional therapy. The additional therapy can be administered prior to, concurrently with, or subsequent to administration of the bispecific antibodies or pharmaceutical compositions described herein. Combined administration can include co-administration, either in a single pharmaceutical formulation or using separate formulations, or consecutive administration in either order but generally within a time period such that all active agents can exert their biological activities simultaneously. A person skilled in the art can readily determine appropriate regimens for administering a pharmaceutical composition described herein and an additional therapy in combination, including the timing and dosing of an additional agent to be used in a combination therapy, based on the needs of the subject being treated.

### 7. Experimental

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

Briefly, results from the studies described below demonstrated that bispecific antibodies bind to both human TNFα and human IL23p19 with high affinities and effectively neutralize the bioactivity of human TNFα and human IL23p19 in vitro and in vivo.

Reference antibodies used in these studies included adalimumab (light chain and heavy chain: SEQ ID NOs: 3 and 4, respectively), golimumab (light chain and heavy chain: SEQ ID NOs: 7 and 8, respectively), ozoralizumab (SEQ ID NO: 9), guselkumab (light chain and heavy chain: SEQ ID NOs: 12 and 13, respectively), and tildrakizumab (light chain and heavy chain: SEQ ID NOs: 16 and 17, respectively).

### 7.1 Example 1: Bispecific Antibody Expression and Purification

Methods: The exemplified bispecific antibodies were expressed and purified as follows. Actively growing Expi CHO-S cells were inoculated into serum-free expression medium and incubated at 37° C. with 8% CO₂ in shaker. The cells were carefully examined for contamination and counted for cell density. Plasmids were mixed with transfection reagent and incubated at room temperature for 2 min. The mixture was added into Expi CHO-s cells and incubated. Enhancer and feed were added into the cells 18-22 h after transfection. Cells were centrifuged to collect supernatants. The supernatants were filtered with 0.22 m microfiltration membrane and loaded into the purification column. The antibody elutes were transferred to dialysis bag and dialyzed by PBS. Samples of the bispecific antibodies were stored at 4° C. for testing; the rest was frozen with liquid nitrogen and stored at −70° C.

### 7.2 Example 2: The Binding Affinity of Bispecific Antibodies to TNFα and IL23p19

Methods: The running buffer (HBS-EP+) was prepared by diluting one volume of the 10× stock with 9 volumes of degassed, filtered MilliQ water. The regeneration buffer (10 nM glycine) was prepared by dissolving glycine in MilliQ water and adjusting the pH to 1.5~1.7. The assay was performed at 25° C. Antibodies were injected as capture on the Series S Sensor Chip Protein A. Recombinant antigen (TNFα or IL23p19) at different concentrations was injected over the surface of flow cell 1 and 2 as the association phase, followed by running buffer as the dissociation phase. The affinity data were obtained through the analysis software.

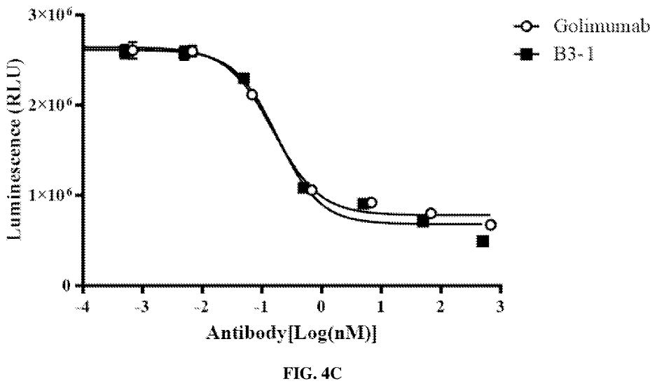

Results and conclusions: As shown in FIG. 2, the exemplary bispecific antibodies disclosed herein bound to both human TNFα and human IL23p19 with high affinities that were comparable to those of the reference antibodies.

### 7.3 Example 3: Simultaneous Binding of TNFα and IL-23p19

Methods: The bispecific antibodies were first captured onto Protein A sensor chip, then 100 nM human IL-23 was injected for 120s to saturate IL-23 binding site of bispecific antibodies. Then human TNFα was injected in a series of concentrations as association phase, followed by running buffer as the dissociation phase. Only one orientation was applied. The affinity data were obtained through the analysis software.

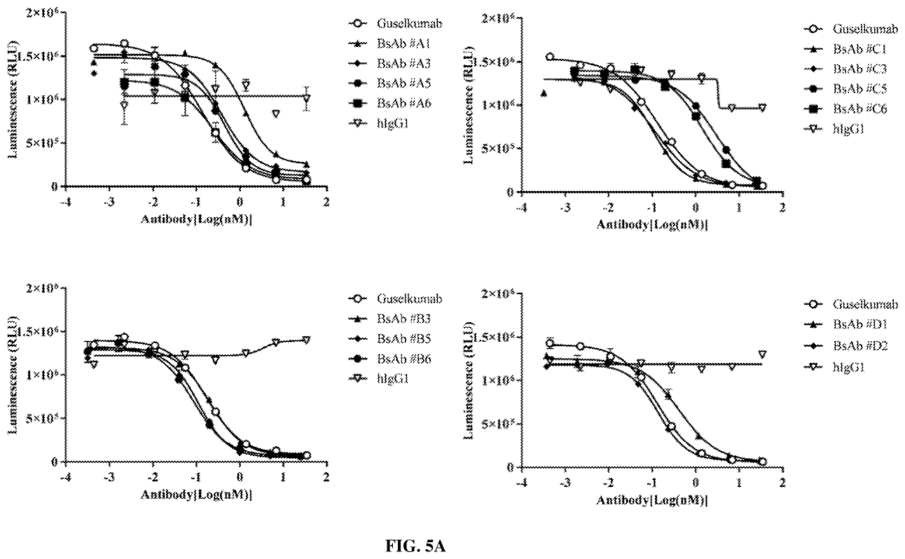

Results and conclusions: As shown in FIG. 3, the exemplary bispecific antibodies disclosed herein simultaneously bound to human TNFα and human IL23p19. Surprisingly, the TNFα binding affinities of the bispecific antibodies that were pre-bound with IL23p19 were comparable to their affinities to TNFα as the single antigen as measured in Example 2, indicating that dual therapeutic purposes could be achieved with these exemplified bispecific antibodies by separately and simultaneously targeting the two antigens, and that these antibodies could serve as a safer and more effective therapeutic option than antibodies targeting a single antigen.

### 7.4 Example 4: Inhibition of TNFα-Induced NF-κB Luciferase Signals in TNFR Reporter Cell Line

Methods: Inhibitory activities of the exemplary bispecific antibodies provided herein on the soluble TNFα signaling pathway were measured through the TNFα neutralization assay using a luciferase reporter cell line. The cell line was engineered to contain NF-κB response element upstream to the luciferase reporter gene. Mediated by TNFα binding to cell surface TNFR, signal cascade activating NF-κB in turn promoted the expression of the luciferase reporter gene. Serially diluted samples of exemplary bispecific antibodies as indicated were pre-incubated with TNFα at ambient temperature in 96-well plates. Following incubation, cells were transferred to wells in the assay plate and incubated. TNFα neutralization potency was determined by luciferase signals.

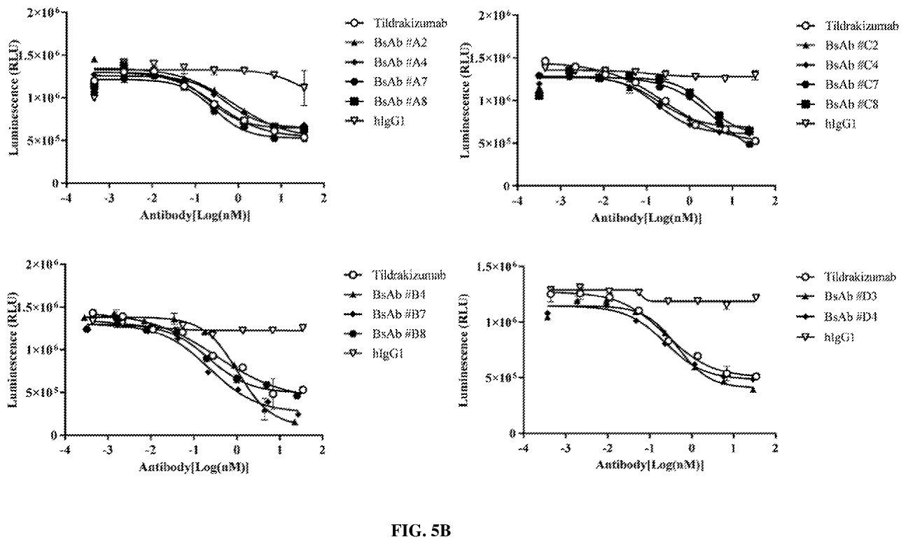

Results and conclusions: As shown in FIG. 4A, FIG. 4B, and FIG. 4C, the exemplary bispecific antibodies disclosed herein effectively inhibited human TNFα-induced NF-xB signaling with high potency. As shown, the inhibition potency of the exemplary bispecific antibodies provided herein was comparable to that observed with the reference antibody.

### 7.5 Example 5: Inhibition of IL-23-Induced STAT3 Phosphorylation Luciferase Signal in IL-23R Reporter Cell Line

Methods: Inhibitory activities of the exemplary bispecific antibodies provided herein on the IL-23 signaling pathway was measured by the IL-23 neutralization assay using a luciferase reporter cell line. Incubation of this reporter cell line with human IL-23 resulted in the phosphorylation of STAT3 mediated by IL-23R/JAK kinase, which was measured using commercial luciferase reporter gene assay reagent. Serially diluted samples of exemplary bispecific antibodies as indicated were pre-incubated with IL-23 at ambient temperature in 96-well plate. Following incubation, cells were transferred to wells in the assay plate and incubated. IL-23 neutralization potency was determined by luciferase signals.

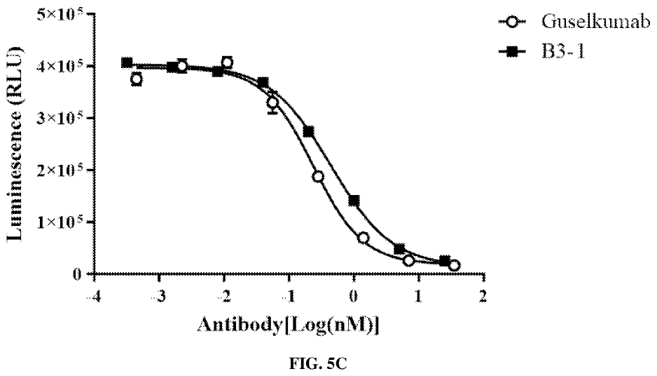

Results and conclusions: As shown in FIG. 5A, FIG. 5B, and FIG. 5C, the exemplary bispecific antibodies disclosed herein effectively inhibited human IL-23-induced STAT3 phosphorylation signaling with high potency. As shown, the inhibition potency of the exemplary bispecific antibodies provided herein was comparable to that observed with the reference antibody.

### 7.6 Example 6: Inhibition of TNFα-Induced U-937 Cells Apoptosis

Methods: U-937 is a pro-monocytic, human myeloid leukemia cell line that naturally expresses the TNF receptor. TNFα induces the caspase-dependent cell apoptosis in U-937 cells. The inhibition of TNFα-induced cell apoptosis is determined by measuring the activity of Caspase 3/7. Serially diluted bispecific antibodies are incubated with U-937 cell for 48h. Following incubation, the luminescent signal is measured by Caspase-Glo” 3/7 assay system.

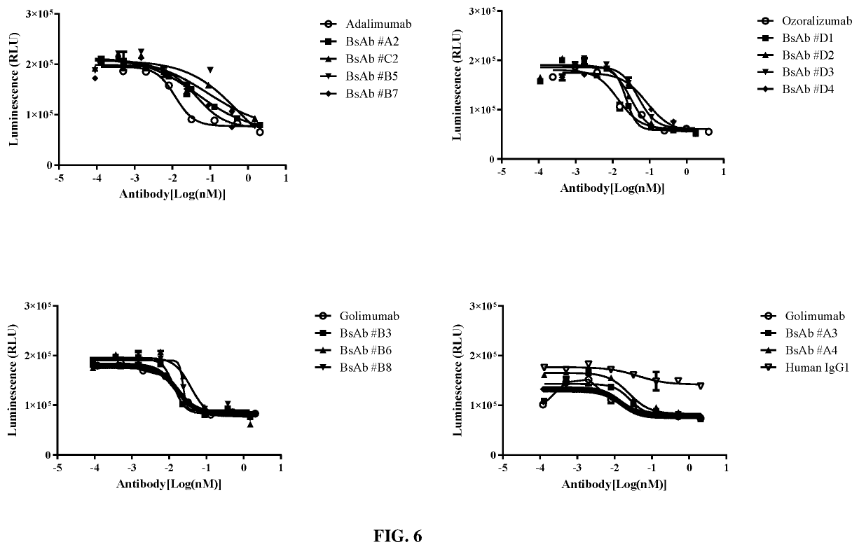

Results and conclusions: As shown in FIG. 6, the exemplary bispecific antibodies disclosed herein effectively inhibited human TNFα-induced U937 cell apoptosis. The inhibition potency of the exemplary bispecific antibodies provided herein was comparable to that observed with the reference antibody.

### 7.7 Example 7: Inhibition of TNFα-Induced Cell Cytotoxicity in L929

Methods: L929 is a mouse fibroblast cell line that naturally expresses the TNF receptor. TNFα induces the L929 cell death due to excessive formation of reactive oxygen intermediates. The inhibition of cell cytotoxicity by bispecific antibodies is determined by measuring the cell viability. Serially diluted bispecific antibodies and TNF-α are pre-incubated and added to L929 cells. The mixture is incubated at 37° C./5% CO₂. Following incubation, the level of cell cytotoxicity is determined by measuring the luminescence signals.

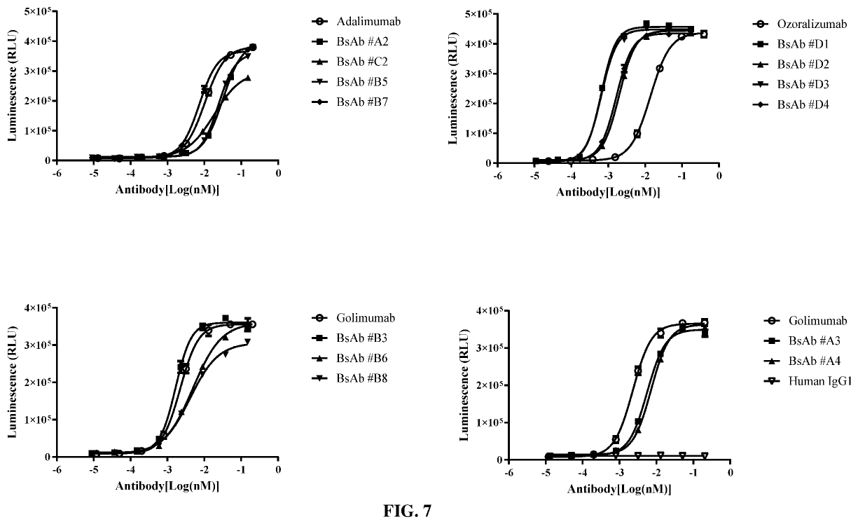

Results and conclusions: As shown in FIG. 7, the exemplary bispecific antibodies disclosed herein effectively inhibited human TNFα-induced L929 cell cytotoxicity. The inhibition potency of the exemplary bispecific antibodies provided herein was comparable to that observed with the reference antibody.

### 7.8 Example 8: Inhibition of TNFα Combination with IL-23-Induced IL-17 Cytokine Production in Human PBMC

Methods: TNFα is known to enhanced IL-17 secretion from Th17 cells. Human PBMC are treated with TNFα and IL-23 containing cytokine mixture that has been preincubated with titrated bispecific antibodies. Cell supernatants are collected and analyzed for IL-17 concentrations by commercial ELISA kits.

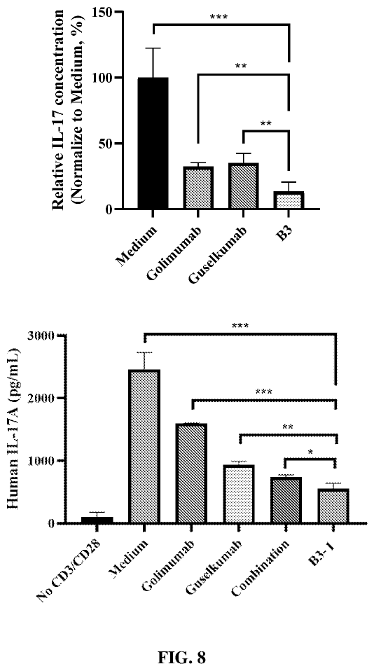

Results and conclusions: As shown in FIG. 8, the exemplary bispecific antibodies disclosed herein effectively inhibited human IL-17 cytokine production induced by TNFα combination with IL-23 in human PBMC. The inhibition potency of the exemplary bispecific antibodies provided herein was better than that observed with the reference antibody.

### 7.9 Example 9: Inhibition of Human TNFα-Induced mIL-6 Production in Mice

Methods: Mice are injected with bispecific antibodies provided herein and then challenged by human TNFα to induce the in vivo production of IL-6. After the challenge, longitudinal serum from whole blood is collected and analyzed for mouse IL-6 levels using a commercial ELISA assay kit.

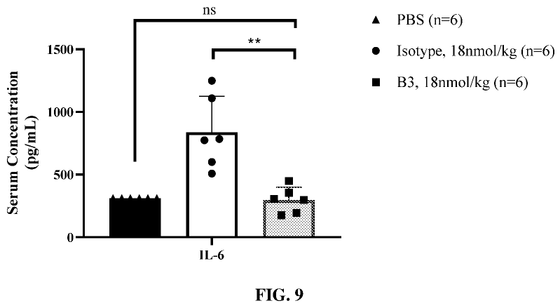

Results and conclusions: As shown in FIG. 9, the exemplary bispecific antibody disclosed herein significantly inhibited in vivo IL-6 production in mouse induced by human TNFα.

### 7.10 Example 10: Inhibition of Human IL-23 Induced Ear Hyperplasia in Mice

Methods: Mice are injected with bispecific antibodies and then intradermally challenged by human IL-23 to induce ear hyperplasia. Ear thickness, ear tissue cytokine and ear histopathology score are evaluated.

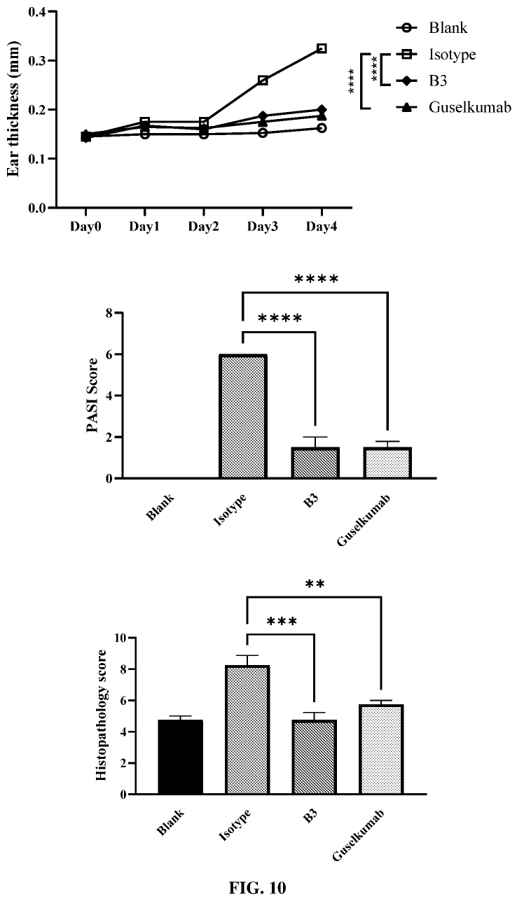

Results and conclusions: As shown in FIG. 10, the exemplary bispecific antibody disclosed herein significantly improved ear thickness, clinical PASI score, and histopathology induced by human IL-23 in the mouse ear hyperplasia model. The inhibition potency of the bispecific antibody provided herein was comparable to that observed with the reference antibody.

As such, compared to monospecific antibodies targeting TNFα or anti-IL23, or the combination thereof, the bispecific antibodies provided herein combine the benefits of additive and/or synergistic therapeutic efficacy, comparable safety, reduced costs, and the improved convenience, safety, and patient compliance as a single agent.

### REFERENCE TO SEQUENCE LISTING SUBMITTED ELECTRONICALLY

This application incorporates by reference a Sequence Listing entitled “[P22406692C].SEQ.XML” created on Sep. 18, 2022 and having a size of 142,259 bytes.

1. A bispecific antibody of any one of bispecific antibodies (A) to (D) that specifically binds to human TNF alpha (TNFα) and to the P19 subunit of human IL-23 (IL23p19), wherein bispecific antibody (A) comprises:
(1) a first light chain (LC1) comprising a first light chain variable domain (VL1) and a first heavy chain constant domain 1 (CH1);
(2) a first heavy chain (HC1) comprising a first heavy chain variable domain (VH1), a first light chain constant (CL) region, and a Knob-Fc region;
(3) a second light chain (LC2) comprising a second light chain variable domain (VL2) and a second CL region; and
(4) a second heavy chain (HC2) comprising a second heavy chain variable domain (VH2), a second CH1 domain, and a Hole-Fc region;
wherein
(i) the VL1/VH1 pair and the VL2/VH2 pair specifically bind to human TNFα and human IL23p19, respectively, or to human IL23p19 and human TNFα, respectively; and
(ii) the Knob-Fc region is a human IgG Fc region variant having a T366W substitution (numbered according to the EU Index); and the Hole-Fc region is a human IgG Fc region variant having a Y407V substitution (numbered according to the EU Index);
bispecific antibody (B) comprises:
(1) a light chain (LC) comprising a first light chain variable domain (VL1) and a CL region; and
(2) a heavy chain (HC) comprising a first heavy chain variable domain (VH1), a heavy chain constant region (CH), a second light chain variable domain (VL2); and a second heavy chain variable domain (VH2);
wherein the VL1/VH1 pair and VL2/VH2 pair specifically bind to human TNFα and human IL23p19, respectively, or to human IL23p19 and human TNFα, respectively;
bispecific antibody (C) comprises:
(1) a light chain (LC) comprising a first light chain variable domain (VL1), a second light chain variable domain (VL2), and a CL region; and
(2) a heavy chain (HC) comprising a first heavy chain variable domain (VH1), a second heavy chain variable domain (VH2), and a CH region;
wherein the VL1/VH1 pair and VL2/VH2 pair specifically bind to human TNFα and human IL23p19, respectively, or to human IL23p19 and human TNFα, respectively;
and bispecific antibody (D) comprises:
(1) a light chain (LC) comprising a light chain variable domain (VL) and a CL region; and
(2) a heavy chain (HC) comprising a heavy chain variable domain (VH) and a CH region;
wherein
(1) the VL/VH pair specifically binds to human IL23p19; and (2) the HC further comprises a single heavy chain variable domain antibody (VHH) that specifically binds to human TNFα.

2. The bispecific antibody of claim 1, wherein
for bispecific antibody (A):
(1) the first CL region is kappa CL (Cκ; SEQ ID NO: 68) or lambda CL (Cλ, SEQ ID NO: 69); and the second CL region is Cκ (SEQ ID NO: 68) or Cλ (SEQ ID NO: 69);
(2) the first CH1 domain and the second CH1 domain are both human IgG1 CH1 domain (SEQ ID NO: 61); or
(3) the Knob-Fc region has the amino acid sequence of SEQ ID NO: 66; and the Hole-Fc region has the amino acid sequence of SEQ ID NO: 67; or any combination of (1)-(3);
for bispecific antibody (B),
(1) the CL region is Cκ (SEQ ID NO: 68) or Cλ (SEQ ID NO: 69); or
(2) the CH region is human IgG1 CH region (SEQ ID NO: 58); or both (1) and (2);
for bispecific antibody (C),
(1) the CL region is Cκ (SEQ ID NO: 68) or Cλ (SEQ ID NO: 69); or
(2) the CH region is human IgG1 CH region (SEQ ID NO: 58); or both (1) and (2);
for bispecific antibody (D),
(1) the CL region is Cκ (SEQ ID NO: 68) or Cλ (SEQ ID NO: 69); or
(2) the CH region is human IgG1 CH region (SEQ ID NO: 58); or both (1) and (2).

3. The bispecific antibody of claim 1, wherein
for bispecific antibody (A), the VL1/VH1 pair specifically binds to human TNFα and the VL2/VH2 pair specifically binds to human IL23p19;
for bispecific antibody (B), the VL1/VH1 pair specifically binds to human TNFα and the VL2/VH2 pair specifically binds to human IL23p19;
for bispecific antibody (C), the VL1/VH1 pair specifically binds to human TNFα and the VL2/VH2 pair specifically binds to human IL23p19;
for bispecific antibody (D), the VL and VH have the amino acid sequences of (1) SEQ ID NOs: 10 and 11, respectively; or (2) SEQ ID NOs: 14 and 15, respectively; and/or, the VHH has the amino acid sequence of SEQ ID NO: 9; and/or, the VHH is linked to the N-terminus of the VH; and/or, the VHH is linked to the C-terminus of the Fc domain.

4. The bispecific antibody of claim 3, wherein
for bispecific antibody (A), the VL1 and VH1 have the amino acid sequences of (1) SEQ ID NOs: 1 and 2, respectively; or (2) SEQ ID NOs: 5 and 6, respectively; or wherein the VL2 and VH2 have the amino acid sequences of (1) SEQ ID NOs: 10 and 11, respectively; or (2) SEQ ID NOs: 14 and 15, respectively; or,
the VL1, VH1, VL2 and VH2 have the amino acid sequences of (1) SEQ ID NOs: 1, 2, 10, and 11, respectively; (2) SEQ ID NOs: 1, 2, 14, and 15, respectively; (3) SEQ ID NOs: 5, 6, 10, and 11, respectively; or (4) SEQ ID NOs: 5, 6, 14, and 15, respectively; or, LC1, HC1, LC2, and HC2 each has at least 90%, at least 95%, at least 98%, at least 99%, or 100% sequence identity to the amino acid sequences of (1) SEQ ID NOs: 24, 25, 12, and 26, respectively; (2) SEQ ID NOs: 27, 28, 12, and 26, respectively; (3) SEQ ID NOs: 24, 25, 16, and 29, respectively; or (4) SEQ ID NOs: 27, 28, 16, and 29, respectively;
for bispecific antibody (B),
the VL1 and VH1 have the amino acid sequences of (1) SEQ ID NOs: 1 and 2, respectively; or (2) SEQ ID NOs: 5 and 6, respectively; or wherein the VL2 and VH2 have the amino acid sequences of (1) SEQ ID NOs: 10 and 11, respectively; or (2) SEQ ID NOs: 14 and 15, respectively; or (3) SEQ ID NOs: 93 and 94, respectively;
or, the VL1, VH1, VL2 and VH2 have the amino acid sequences of (1) SEQ ID NOs: 1, 2, 10, and 11, respectively; (2) SEQ ID NOs: 1, 2, 14, and 15, respectively; (3) SEQ ID NOs: 1, 2, 93, and 94, respectively; (4) SEQ ID NOs: 5, 6, 10, and 11, respectively; or (5) SEQ ID NOs: 5, 6, 14, and 15, respectively; (6) SEQ ID NOs: 5, 6, 93, and 94, respectively;
or, the LC and HC each has at least 90%, at least 95%, at least 98%, at least 99%, or 100% sequence identity to the amino acid sequences of (1) SEQ ID NOs: 3 and 30, respectively; (2) SEQ ID NOs: 3 and 31 respectively; (3) SEQ ID NOs: 7 and 32, respectively; (4) SEQ ID NOs: 7 and 33, respectively; or (5) SEQ ID NOs: 7 and 92, respectively;
for bispecific antibody (C),
the VL1 and VH1 have the amino acid sequences of (1) SEQ ID NOs: 1 and 2, respectively; or (2) SEQ ID NOs: 5 and 6, respectively; or wherein VL2 and VH2 have the amino acid sequences of (1) SEQ ID NOs: 10 and 11, respectively; or (2) SEQ ID NOs: 14 and 15, respectively or, the VL1, VH1, VL2 and VH2 have the amino acid sequences of (1) SEQ ID NOs: 1, 2, 10, and 11, respectively; (2) SEQ ID NOs: 1, 2, 14, and 15, respectively; (3) SEQ ID NOs: 5, 6, 10, and 11, respectively; or (4) SEQ ID NOs: 5, 6, 14, and 15, respectively;
or, the LC and HC each has at least 90%, at least 95%, at least 98%, at least 99%, or 100% sequence identity to the amino acid sequences of (1) SEQ ID NOs: 46 and 47, respectively; (2) SEQ ID NOs: 48 and 49 respectively; (3) SEQ ID NOs: 50 and 51, respectively; or (4) SEQ ID NOs: 52 and 53, respectively;
for bispecific antibody (D),
the LC and HC each has at least 90%, at least 95%, at least 98%, at least 99%, or 100% sequence identity to the amino acid sequences of (1) SEQ ID NOs: 12 and 54, respectively; or (2) SEQ ID NOs: 16 and 56 respectively; and/or, when the VHH is linked to the C-terminus of the Fc domain, the LC and the HC each has at least 90%, at least 95%, at least 98%, at least 99%, or 100% sequence identity to the amino acid sequences of (1) SEQ ID NOs: 12 and 55, respectively; or (2) SEQ ID NOs: 16 and 57, respectively.

5-6. (canceled)

7. The bispecific antibody of claim 1, wherein
for bispecific antibody (A), the VL1/VH1 pair specifically binds to human IL23p19 and the VL2/VH2 pair specifically binds to human TNFα;
for bispecific antibody (B), the VL1/VH1 pair specifically binds to human IL23p19 and the VL2/VH2 pair specifically binds to human TNFα;
for bispecific antibody (C), the VL1/VH1 pair specifically binds to human IL23p19 and the VL2/VH2 pair specifically binds to human TNFα.

8. The bispecific antibody of claim 7, wherein for bispecific antibody (A),
VL1 and VH1 have the amino acid sequences of (1) SEQ ID NOs: 10 and 11, respectively; or (2) SEQ ID NOs: 14 and 15, respectively; or wherein VL2 and VH2 have the amino acid sequences of (1) SEQ ID NOs: 1 and 2, respectively; or (2) SEQ ID NOs: 5 and 6, respectively; or,
VL1, VH1, VL2 and VH2 have the amino acid sequences of (1) SEQ ID NOs: 10, 11, 1, and 2, respectively; (2) SEQ ID NOs: 10, 11, 5, and 6, respectively; (3) SEQ ID NOs: 14, 15, 1, and 2, respectively; or (4) SEQ ID NOs: 14, 15, 5, and 6, respectively; or,
LC1, HC1, LC2, and HC2 each has at least 90%, at least 95%, at least 98%, at least 99%, or 100% sequence identity to the amino acid sequences of (1) SEQ ID NOs: 18, 19, 3, and 20, respectively; (2) SEQ ID NOs: 21, 22, 3, and 20 respectively; (3) SEQ ID NOs: 18, 19, 7 and 23, respectively; or (4) SEQ ID NOs: 21, 22, 7, and 23, respectively;
for bispecific antibody (B),
the VL1 and VH1 have the amino acid sequences of (1) SEQ ID NOs: 10 and 11, respectively; or (2) SEQ ID NOs: 14 and 15, respectively; or wherein VL2 and VH2 have the amino acid sequences of (1) SEQ ID NOs: 1 and 2, respectively; or (2) SEQ ID NOs: 5 and 6, respectively or, the VL1, VH1, VL2 and VH2 have the amino acid sequences of (1) SEQ ID NOs: 10, 11, 1, and 2, respectively; (2) SEQ ID NOs: 10, 11, 5, and 6, respectively; (3) SEQ ID NOs: 14, 15, 1, and 2, respectively; or (4) SEQ ID NOs: 14, 15, 5, and 6, respectively;
or, the LC and HC each has at least 90%, at least 95%, at least 98%, at least 99%, or 100% sequence identity to the amino acid sequences of (1) SEQ ID NOs: 12 and 34, respectively; (2) SEQ ID NOs: 12 and 35, respectively; (3) SEQ ID NOs: 16 and 36, respectively; or (4) SEQ ID NOs: 16 and 37, respectively;
for bispecific antibody (C),
the VL1 and VH1 have the amino acid sequences of (1) SEQ ID NOs: 10 and 11, respectively; or (2) SEQ ID NOs: 14 and 15, respectively; or wherein VL2 and VH2 have the amino acid sequences of (1) SEQ ID NOs: 1 and 2, respectively; or (2) SEQ ID NOs: 5 and 6, respectively;
or, the VL1, VH1, VL2 and VH2 have the amino acid sequences of (1) SEQ ID NOs: 10, 11, 1, and 2, respectively; (2) SEQ ID NOs: 10, 11, 5, and 6, respectively; (3) SEQ ID NOs: 14, 15, 1, and 2, respectively, or (4) SEQ ID NOs: 14, 15, 5, and 6, respectively;
or, the LC and HC each has at least 90%, at least 95%, at least 98%, at least 99%, or 100% sequence identity to the amino acid sequences of (1) SEQ ID NOs: 38 and 39, respectively; (2) SEQ ID NOs: 40 and 41, respectively; (3) SEQ ID NOs: 42 and 43, respectively; or (4) SEQ ID NOs: 44 and 45, respectively.

9-38. (canceled)

39. A polynucleotide encoding the LC1, the LC2, the HC1, the HC2, or any combination thereof, of the bispecific antibody (A),
or encoding the LC, the HC, or both the LC and HC of any one of bispecific antibodies (B) to (D), of claim 1.

40. (canceled)

41. A vector comprising the polynucleotide of claim 39.

42. A cell comprising (a) a polynucleotide that encodes the LC1, LC2, HC1, and HC2, or (b) a plurality of the polynucleotides that collectively encode the LC1, LC2, HC1, and HC2;
or a cell comprising (a′) a polynucleotide that encode both the LC and HC, or (b′) a first polynucleotide that encodes the LC and a second polynucleotide that encodes the HC;
wherein the polynucleotide in (a), (b) and (a′), the first nucleotide and the second nucleotide in (b′) are as defined in claim 39.

43. (canceled)

44. A pharmaceutical composition comprising the bispecific antibody of claim 1, a polynucleotide, and/or a cell, and optionally a pharmaceutically acceptable carrier;
wherein the polynucleotide encodes:
(I) the LC1, the LC2, the HC1, the HC2, or any combination thereof, of (A); or,
(II) the LC, the HC, or both the LC and HC of (B) to (D),
of the bispecific antibody:
and the cell comprises the polynucleotide or a plurality of the polynucleotides.

45. A method of reducing TNFα and/or IL23p19-associated autoimmunity or inflammation, treating an autoimmune disease or an inflammatory disease in a subject in need thereof comprising administering to the subject a therapeutically effective amount of the bispecific antibody of claim 1, a polynucleotide, a cell and/or a pharmaceutical composition;
wherein the polynucleotide encodes:
(I) the LC1, the LC2, the HC1, the HC2, or any combination thereof, of (A); or,
(II) the LC, the HC, or both the LC and HC of (B) to (D),
of the bispecific antibody:
the cell comprises the polynucleotide or a plurality of the polynucleotides;
and the pharmaceutical composition comprises the bispecific antibody, the polynucleotide, and/or the cell, and optionally a pharmaceutically acceptable carrier.

46. The method of claim 45, wherein the subject has an autoimmune disease or an inflammatory disease;
and/or, the subject is a human.

47-54. (canceled)

55. A method of producing a bispecific antibody that specifically binds to human TNFα and to human IL23p19 by expressing the polynucleotide or the plurality of polynucleotides in the cell of claim 42.
